(19) United States
(12) Patent Application Publication
(10) Pub. No.: US 2025/0282713 A1
(43) Pub. Date: Sep. 11, 2025

(54) EFFICIENT CAPTURE AND RELEASE OF THE RARE-EARTH ELEMENTS USING COVALENT ORGANIC FRAMEWORKS

(71) Applicant: IOWA STATE UNIVERSITY RESEARCH FOUNDATION, INC., Ames, IA (US)

(72) Inventors: Levi M. STANLEY, Ames, IA (US); Wenyu HUANG, Ames, IA (US); Alexander VOLKOV, Ames, IA (US); Puranjan CHATTERJEE, Ames, IA (US); Aaron J. ROSSINI, Ames, IA (US); Jiashan MI, Ames, IA (US)

(21) Appl. No.: 19/073,642

(22) Filed: Mar. 7, 2025

Related U.S. Application Data
(60) Provisional application No. 63/563,036, filed on Mar. 8, 2024.

(51) Int. Cl. C07C 233/88; B01J 20/22; C22B 59/00

(52) U.S. Cl. CPC C07C 233/88; B01J 20/22; C22B 59/00

(57) ABSTRACT

The present disclosure is directed to a covalent organic framework comprising repeating units of a moiety comprising Formula (I):

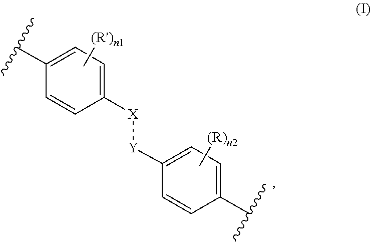

wherein X, Y, R, R′, n₁, and n₂ are as described herein. The present disclosure is also directed to a method of making the covalent organic frameworks and a method of separating rare-earth elements using the covalent organic frameworks.

This application claims the benefit of U.S. Provisional Patent Application Ser. No. 63/563,036, filed Mar. 8, 2024, which is hereby incorporated by reference in its entirety.

This invention was made with government support under DE-AC02-07CH11358 awarded by Department of Energy. The government has certain rights in the invention.

### FIELD

The present application relates to efficient capture and release of the rare-earth elements using covalent organic frameworks.

### BACKGROUND

Rare-earth elements (REEs) are essential components in the manufacturing of semiconductors and permanent magnets. Important applications of REEs include consumer electronics, transportation, defense, power generation, health care, and petroleum refinement refining (Rare Earth Elements, Department of Energy Office of Fossil Energy and Carbon Management (2017), www.energy.gov/fecm/rare-earth-elements (accessed 2023 Oct. 20)). While the global demand for REEs is increasing, the supply is still limited (Rare Earths Statistics and Information, United States Geological Survey (2023), www.usgs.gov/centers/national-minerals-information-center/rare-earths-statistics-and-information (accessed 2023 Oct. 20). Tremendous experimental and theoretical efforts have been invested in the last two decades to develop soluble ligands for binding REE ions using liquid-liquid extraction (Poole et al., “Computational Modeling of Diphosphine Oxide and Diglycolamide Ligand Complexation to Lanthanides and Extraction from Acidic Media,” In Penchoff, eds., Rare Earth Elements and Actinides: Progress in Computational Science Applications, ACS Symposium Series, Vol. 1388; Washington, DC: American Chemical Society, pp. 347-363 (2021); Higgins et al., “Coordination Chemistry-Driven Approaches to Rare Earth Element Separations,” Acc. Chem. Res. 55(18):2616-2627 (2022)). However, liquid-liquid extractions generate significant chemical waste, while the homogeneous ligands are difficult to recycle, dramatically increasing extraction costs (Pereira Neves et al., “Liquid-Liquid Extraction of Rare Earth Elements Using Systems that are More Environmentally Friendly: Advances, Challenges and Perspectives,” Sep. Purif Technol. 282 Part B:120064 (2022)). More sustainable protocols and materials for the extraction and recycling of REEs are crucial to ensure their continuous supply. In solid-liquid extractions, on the other hand, a solid adsorbent is utilized to capture REE ions from the solution (Hidayah and Abidin, “The Evolution of Mineral Processing in Extraction of Rare Earth Elements Using Solid-Liquid Extraction over Liquid-Liquid Extraction: A Review,” Miner. Eng. 112:103-113 (2017)). Because the solid adsorbent is easily separated from the liquid phase by filtration, it can potentially be recycled multiple times and eliminate the use of organic solvents, reducing the overall cost of extraction. Many solid adsorbents have been explored for REE capture, including silica (Hu et al., “Highly Efficient and Selective Recovery of Rare Earth Elements Using Mesoporous Silica Functionalized by Preorganized Chelating Ligands,” ACS Appl. Mater. Interfaces, 9(44):38584-38593 (2017); Callura et al., “Selective Adsorption of Rare Earth Elements onto Functionalized Silica Particles,” Green Chem., 20(7):1515-1526 (2018); Ashour et al., “DTPA-Functionalized Silica Nano- and Microparticles for Adsorption and Chromatographic Separation of Rare Earth Elements,” ACS Sustain. Chem. Eng., 6(5):6889-6900 (2018); Hu et al., “Size-Selective Separation of Rare Earth Elements Using Functionalized Mesoporous Silica Materials,” ACS Appl. Mater. Interfaces, 11(26):23681-23691 (2019)), zeolites (Guzzinati et al., “Formation of Supramolecular Clusters at the Interface of Zeolite X Following the Adsorption of Rare-Earth Cations and Their Impact on the Macroscopic Properties of the Zeolite,” Chem Phys Chem, 19(17):2208-2217 (2018); Barros et al., “Recovery of Rare Earth Elements from Wastewater Towards a Circular Economy,” Molecules, 24(6):1005 (2019); Ji and Zhang, “Adsorption of Cerium (III) by Zeolites Synthesized from Kaolinite after Rare Earth Elements (REEs) Recovery,” Chemosphere, 303(1):134941 (2022); Chatterjee et al., “Capturing Rare-Earth Elements by Synthetic Aluminosilicate MCM-22: Mechanistic Understanding of Yb(III) Capture,” ACS Appl. Mater. Interfaces, 15(46): 54192-54201 (2023)), metal-oxides (Liu et al., “Efficient and Rapid Adsorption of Rare Earth Elements from Water by Magnetic Fe₃O₄/MnO₂ Decorated Reduced Graphene Oxide. J. Mol. Liq., 313:113510 (2020); Sarmadi et al., “Highly Mesoporous Hybrid Transition Metal Oxide Nanowires for Enhanced Adsorption of Rare Earth Elements from Wastewater,” Inorg. Chem., 60(1):175-184 (2021)), organic polymers (Ma et al., “Functional Fibrous Materials-Based Adsorbents for Uranium Adsorption and Environmental Remediation,” Chem. Eng. J., 390:124597 (2020); Maruthapandi et al., “Nitrogen-Enriched Porous Benzimidazole-Linked Polymeric Network for the Adsorption of La (III), Ce (III), and Nd (III),” J. Phys. Chem. C, 124(11):6206-6214 (2020); Ravi et al., “Novel Benzylphosphate-Based Covalent Porous Organic Polymers for the Effective Capture of Rare Earth Elements from Aqueous Solutions,” J. Hazard. Mater., 424 (Pt A):127356 (2022); Zhang and Gao, “Polymeric Materials for Rare Earth Elements Recovery,” Gels, 9(10):775 (2023)), metal-organic frameworks (Lee et al., “Selective Adsorption of Rare Earth Elements over Functionalized Cr-MIL-101,” ACS Appl. Mater. Interfaces, 10(28):23918-23927 (2018); Peng et al., “A Versatile MOF-Based Trap for Heavy Metal Ion Capture and Dispersion,” Nat. Commun., 9:187 (2018); Fonseka et al., “Selective Recovery of Rare Earth Elements from Mine Ore by Cr-MIL Metal-Organic Frameworks,” ACS Sustain. Chem. Eng., 9(50):16896-16904 (2021); Zhao et al., “Nanoporous Metal-Organic Framework Adsorbent Constructed via Ligand Tailoring for Rare-Earth Metal Ion Recovery,” ACS Appl. Nano Mater, 6(24):22865-22875 (2023)), and graphene oxides (Li et al., “High Efficient Separation of U(VI) and Th(IV) From Rare Earth Elements in Strong Acidic Solution by Selective Sorption on Phenanthroline Diamide Functionalized Graphene Oxide,” Chem. Eng. J., 332:340-350 (2018); Zhao et al., “Behavior and Mechanism of Graphene Oxide-tris(4-aminophenyl)amine Composites in Adsorption of Rare Earth Elements,” J. Rare Earths, 39(1):90-97 (2021); Chen et al., “Selective Adsorption of Rare Earth Elements by Zn-BDC MOF/Graphene Oxide Nanocomposites Synthesized via In Situ Interlayer-Confined Strategy,” Ind. Eng. Chem. Res., 61(4):1841-1849 (2022); Bao et al., “Highly Efficient Recovery of Heavy Rare Earth Elements by Using an Amino-Functionalized Magnetic Graphene Oxide with Acid and Base Resistance,” J. Hazard. Mater., 424:127370 (2022)). However, the existing materials tend to lack either the required efficiency or customizable molecular functionality and, oftentimes, cannot be easily recycled. To address these challenges, new solid adsorbent materials must be developed that will combine the efficiency of the homogeneous ligands with the recyclability of the heterogeneous materials. Next-generation REE adsorbents must possess tunable molecular structures and robust chemical backbones to achieve efficient REE capture and high stability for multiple extraction cycles.

In this regard, one class of material with the requisite properties for solid-liquid extraction of REEs is covalent organic frameworks (COFs). COFs are crystalline organic solids that combine attractive physical properties, such as defined crystal structure, chemical stability, and permanent porosity (Huang et al., “Covalent Organic Frameworks: A Materials Platform for Structural and Functional Designs,” Nat. Rev. Mater., 1:16068 (2016); Diercks et al., “The Atom, the Molecule, and the Covalent Organic Framework,” Science, 355(6328):923-931 (2017); Geng et al., “Covalent Organic Frameworks: Design, Synthesis, and Functions,” Chem. Rev., 120(16):8814-8933 (2020)). Since the inception of COFs almost two decades ago (Cote et al., “Porous, Crystalline, Covalent Organic Frameworks,” Science, 310(5751):1166-1170 (2005)), the scientific community has been vigorously exploring chemical space to discover architectures with new morphologies, pore sizes, and relevant functionalities. The imine bond has proven to be one of the most accessible linkages in COFs, as evidenced by the vast library of imine COFs produced, studied, and characterized. In fact, using the latest COF database (Ongari et al., “Too Many Materials and Too Many Applications: An Experimental Problem Waiting for a Computational Solution,” ACS Cent. Sci., 6(11):1890-1900 (2020)), more than 60% of all unique COF skeletons are linked by an imine bond. This success can be attributed to the imine bond being a near-ideal choice for dynamic covalent chemistry (Corbett et al., “Dynamic Combinatorial Chemistry,” Chem. Rev., 106(9):3652-3711 (2006); Hu et al., “Applications of Dynamic Covalent Chemistry Concept toward Tailored Covalent Organic Framework Nanomaterials: A Review,” ACS Appl. Nano Mater., 3(7):6239-6269 (2020)), a principle that describes the controlled growth of supramolecular assemblies. In addition to the imine bond serving as a bridge between the two building blocks, it can also be utilized as a synthon to introduce new functional groups in the COFs (Qian et al., “Imine and Imine-Derived Linkages in Two-Dimensional Covalent Organic Frameworks,” Nat. Rev. Chem., 6(12):881-898 (2022)) (FIG. 1). Examples of imine bond transformations in COFs include cyclizations to form oxazole (Pyles et al., “Synthesis of Benzobisoxazole-Linked Two-Dimensional Covalent Organic Frameworks and Their Carbon Dioxide Capture Properties,” ACS Macro Lett., 5(9):1055-1058 (2016); Waller et al., “Conversion of Imine to Oxazole and Thiazole Linkages in Covalent Organic Frameworks,” J. Am. Chem. Soc., 140(29):9099-9103 (2018)), thiazole (Waller et al., “Conversion of Imine to Oxazole and Thiazole Linkages in Covalent Organic Frameworks,” J. Am. Chem. Soc., 140(29):9099-9103 (2018); Wang et al., “Synthesis of Stable Thiazole-Linked Covalent Organic Frameworks via a Multicomponent Reaction,” J. Am. Chem. Soc., 142(25):11131-11138 (2020)), imidazole (Wang et al., “Constructing Robust Covalent Organic Frameworks via Multicomponent Reactions,” J. Am. Chem. Soc., 141(45):18004-18008 (2019); Yang et al., “Covalent Organic Frameworks with Irreversible Linkages via Reductive Cyclization of Imines,” J. Am. Chem. Soc., 144(22):9827-9835 (2022)), indazole (Yang et al., “Covalent Organic Frameworks with Irreversible Linkages via Reductive Cyclization of Imines,” J. Am. Chem. Soc., 144(22):9827-9835 (2022)), quinoline (Li et al., “Facile Transformation of Imine Covalent Organic Frameworks into Ultrastable Crystalline Porous Aromatic Frameworks,” Nat. Commun., 9:2998 (2018); Zhao et al., “Construction of Ultrastable Nonsubstituted Quinoline-Bridged Covalent Organic Frameworks via Rhodium-Catalyzed Dehydrogenative Annulation,” Angew. Chem., Int. Ed., 61(41):e202208833 (2022); Li et al., “Construction of Covalent Organic Frameworks via Three-Component One-Pot Strecker and Povarov Reactions,” J. Am. Chem. Soc. 142(14):6521-6526 (2020); Yao et al., “Sulfonic Acid and Ionic Liquid Functionalized Covalent Organic Framework for Efficient Catalysis of the Biginelli Reaction,” J. Org. Chem., 86(3):3024-3032 (2021)), tetrahydroquinoline (Li et al., “Asymmetric Photocatalysis over Robust Covalent Organic Frameworks with Tetrahydroquinoline Linkage,” Chinese J. Catal., 41(8):1288-1297 (2020); Lyu et al., “Covalent Organic Frameworks for Carbon Dioxide Capture from Air,” J. Am. Chem. Soc., 144(28):12989-12995 (2022)), chromenoquinoline (Feng et al., “Fused-Ring-Linked Covalent Organic Frameworks,” J. Am. Chem. Soc. 144(14):6594-6603 (2022); Ren et al., “Constructing Stable Chromenoquinoline-Based Covalent Organic Frameworks via Intramolecular Povarov Reaction,” J. Am. Chem. Soc., 144(6):2488-2494 (2022)), thieno[3,2-c]pyridine (Wang et al., “Construction of Fully Conjugated Covalent Organic Frameworks via Facile Linkage Conversion for Efficient Photoenzymatic Catalysis,” J. Am. Chem. Soc., 142(13):5958-5963 (2020)), and carbamates (Lyle et al., “Multistep Solid-State Organic Synthesis of Carbamate-Linked Covalent Organic Frameworks,” J. Am. Chem. Soc., 141(28):11253-11258 (2019)), oxidations to amides (Waller et al., “Chemical Conversion of Linkages in Covalent Organic Frameworks,” J. Am. Chem. Soc., 138(48):15519-15522 (2016); Zhou et al., “A Facile, Efficient, and General Synthetic Method to Amide-Linked Covalent Organic Frameworks,” J. Am. Chem. Soc., 144(3):1138-1143 (2022)), reductions to amines (Liu et al., “Covalent Organic Frameworks Linked by Amine Bonding for Concerted Electrochemical Reduction of CO₂,” Chem, 4(7):1696-1709 (2018); Grunenberg et al., “Amine-Linked Covalent Organic Frameworks as a Platform for Postsynthetic Structure Interconversion and Pore-Wall Modification,” J. Am. Chem. Soc., 143(9):3430-3438 (2021)), asymmetric additions (Li et al., “Construction of Covalent Organic Frameworks via Three-Component One-Pot Strecker and Povarov Reactions,” J. Am. Chem. Soc. 142(14):6521-6526 (2020); Wang et al., “Catalytic Asymmetric Synthesis of Chiral Covalent Organic Frameworks from Prochiral Monomers for Heterogeneous Asymmetric Catalysis,” J. Am. Chem. Soc., 142(40):16915-16920 (2020); Lu et al., “Asymmetric Hydrophosphonylation of Imines to Construct Highly Stable Covalent Organic Frameworks with Efficient Intrinsic Proton Conductivity,” J. Am. Chem. Soc., 144(22):9624-9633 (2022)), multicomponent reactions (Wang et al., “Constructing Robust Covalent Organic Frameworks via Multicomponent Reactions,” J. Am. Chem. Soc., 141(45):18004-18008 (2019); Yao et al., “Sulfonic Acid and Ionic Liquid Functionalized Covalent Organic Framework for Efficient Catalysis of the Biginelli Reaction,” J. Org. Chem., 86(3):3024-3032 (2021); Liu et al., “Pyrimidazole-Based Covalent Organic Frameworks: Integrating Functionality and Ultrastability via Isocyanide Chemistry,” J. Am. Chem. Soc., 142(50):20956-20961 (2020)), conversions via linker exchange (Qian et al., “Irreversible Amide-Linked Covalent Organic Framework for Selective and Ultrafast Gold Recovery,” Angew. Chem., Int. Ed., 59(40):17607-17613 (2020); Zhou et al., “Toward Azo-linked Covalent Organic Frameworks by Developing Linkage Chemistry via Linker Exchange,” Nat. Commun., 13:2180 (2022)), and boron complexation (Peng et al., “Intramolecular Hydrogen Bonding-Based Topology Regulation of Two-Dimensional Covalent Organic Frameworks,” J. Am. Chem. Soc., 142(30):13162-13169 (2020)). These transformations are considered a part of the post-synthetic modification (PSM) strategy in COFs. Generally, PSMs improve the chemical stability of the COF backbones and, in some cases, provide additional functionality tailored to a specific application. Unfortunately, most of the established PSMs in COFs offer little to no leeway for further customization of the given reticular template due to the limited access to functional group handles.

The importance of having a reliable and general strategy to introduce a variety of functional group handles in COFs becomes critical as the demand for more elaborate applications of these materials is growing. Alternatively to PSM, the bottom-up design strategy attempts to address this demand in several COF systems (Qian et al., “Bottom-up Synthesis of Chiral Covalent Organic Frameworks and Their Bound Capillaries for Chiral Separation,” Nat. Commun., 7:12104 (2016); Han et al., “Chiral Covalent Organic Frameworks with High Chemical Stability for Heterogeneous Asymmetric Catalysis,” J. Am. Chem. Soc., 139(25):8693-8697 (2017); Wang et al., “Divergent Synthesis of Chiral Covalent Organic Frameworks,” Angew. Chem., Int. Ed., 58(28):9443-9447 (2019)). The bottom-up design strategy employs linkers with built-in scaffolds, which are directly used to fabricate functional COFs. More customizable linker designs incorporate handles for PSM that can be decorated with the molecules of interest after the successful crystallization of the framework (Nagai et al., “Pore Surface Engineering in Covalent Organic Frameworks,” Nat. Commun., 2:536 (2011); Xu et al., “Catalytic Covalent Organic Frameworks via Pore Surface Engineering,” Chem Comm, 50(11):1292-1294 (2014); Xu et al., “Stable, Crystalline, Porous, Covalent Organic Frameworks as a Platform for Chiral Organocatalysts,” Nat. Chem, 7(11):905-912 (2015)). Despite obvious synthetic advantages, such as precise control of the linker design or a tunable density of the active sites, it is often difficult to obtain crystalline porous materials with more sophisticated functional scaffolds. Due to the intricate interplay between the steric and electronic properties of the linker, its solubility, and chemical compatibility with the synthetic protocols, extensive optimization efforts are often required and do not always yield crystalline porous materials (Zhang et al., “Hydrazone-Linked Heptazine Polymeric Carbon Nitrides for Synergistic Visible-Light-Driven Catalysis,” Eur. J. Chem., 26(33):7358-7364 (2020)). COFs with complex linker designs tend to produce staggered structures (Emmerling et al., “Interlayer Interactions as Design Tool for Large-Pore COFs,” J. Am. Chem. Soc., 143(38):15711-15722 (2021); Zhuo et al., “Chiral Carboxyl-Functionalized Covalent Organic Framework for Enantioselective Adsorption of Amino Acids,” ACS Appl. Mater. Interfaces, 13(26):31059-31065 (2021); Li et al., “Combination of a Metal-N-Heterocyclic-Carbene Catalyst and a Chiral Aminocatalyst within a Covalent Organic Framework: a Powerful Cooperative Approach for Relay Asymmetric Catalysis,” Inorg. Chem., 61(5):2455-2462 (2022)), dramatically reducing the effective pore volume and accessibility to the interior surface of the material. These challenges become more pronounced with larger-pore COF systems, where the requirement for linker planarity becomes crucial for the controlled ordering of the material (Mu et al., “Covalent Organic Frameworks with Record Pore Apertures,” J. Am. Chem. Soc., 144(11):5145-5154 (2022)). Satisfying these conditions for successful COF crystallization also imposes an additional synthetic strain on the streamlined production of the linker precursors.

Connected entirely through covalent bonds, COFs are assembled from customizable building blocks into porous organic solids with a defined crystal lattice and high surface area (Diercks and Yaghi, “The Atom, the Molecule, and the Covalent Organic Framework,” Science, 355(6328):923-931 (2017)). Despite coming in a variety of topological designs and reticular dimensions, COFs have been underexplored for REE recovery, with only a few promising reports on Sc (Yuan et al., “Selective Scandium Ion Capture through Coordination Templating in a Covalent Organic Framework,” Nat. Chem., 15(11):1599-1606 (2023)), La (Lu et al., “Postsynthetic Functionalization of Three-Dimensional Covalent Organic Frameworks for Selective Extraction of Lanthanide Ions,” Angew. Chem., Int. Ed., 57(21):6042-6048 (2018); Li et al., “Nanoporous Sulfonic Covalent Organic Frameworks for Selective Adsorption and Separation of Lanthanide Elements,” ACS Appl. Nano Mater., 6(4):2498-2506 (2023)), U (Cui et al., “Regenerable and Stable sp² Carbon-Conjugated Covalent Organic Frameworks for Selective Detection and Extraction of Uranium,” Nat. Commun., 11:436 (2020); Cui et al., “Regenerable Covalent Organic Frameworks for Photo-enhanced Uranium Adsorption from Seawater,” Angew. Chem., Int. Ed., 59(40):17684-17690 (2020); Hao et al., “Modulating Uranium Extraction Performance of Multivariate Covalent Organic Frameworks through Donor-Acceptor Linkers and Amidoxime Nanotraps,” J. Am. Chem. Soc., 3(1):239-251 (2023)), and Th (Xiong et al., “Selective Extraction of Thorium from Uranium and Rare Earth Elements Using Sulfonated Covalent Organic Framework and its Membrane Derivate,” Chem. Eng. J., 384:123240 (2020); Liu et al., “Selective Entrapment of Thorium Using a Three-Dimensional Covalent Organic Framework and its Interaction Mechanism Study,” Sep. Purif Technol., 296:121413 (2022); Liu et al., “Efficient and Selective Capture of Thorium Ions by a Covalent Organic Framework,” Nat. Commun., 14:5097 (2023)) extraction. The two main challenges associated with the streamlined production of COFs tailored to efficient REE extraction are (1) limited molecular diversity of the building blocks suitable for the introduction of REE-bindings scaffolds and (2) chemical lability of the COF linkages in the context of aqueous and low-pH conditions used in REE extraction applications.

The present disclosure is directed to overcoming these and other deficiencies in the art.

### SUMMARY

One aspect of the present disclosure relates to a covalent organic framework comprising repeating units of a moiety comprising Formula (I):

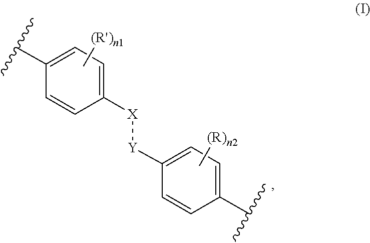

where
--- is a single or a double bond;
X is independently selected at each occurrence thereof from N or N(R³), wherein at least one X is N(R³);
Y is independently selected at each occurrence thereof from C(R¹)(R²) or C(R¹);
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R¹ is H;
R² is optional and, if present, is —C(O)NR⁴;
R³ is —C(O)R⁵;
R⁴ is independently selected at each occurrence thereof from the group consisting of cycloalkyl, —(CH₂)ₘC(O)OC₁₋₆ alkyl, and —(CH₂)ₘC(O)NH(CH₂)ₖNH₂, wherein —(CH₂)ₘC(O)NH(CH₂)ₖNH₂ can be optionally substituted 1 to 3 times with R⁷;
R⁵ is selected from the group consisting of C₁₋₁₂ alkyl, C₂₋₁₂ alkenyl, or C₂₋₁₂ alkynyl, wherein C₁₋₁₂ alkyl can be optionally substituted 1 to 3 times with R⁶;
R⁶ is independently selected at each occurrence thereof from N₃, —C(O)OC₁₋₆ alkyl, —C(O)NH(CH₂)ₖNH₂, wherein —C(O)NH(CH₂)ₖNH₂ can be optionally substituted 1 to 3 times with R⁷;
R⁷ is —(CH₂)ₖ₁NH₂ optionally substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5, or 6; and
k₇ is 1, 2, 3, 4, 5, or 6.

Another aspect of the present disclosure relates to method of separating rare-earth elements. This method comprises providing a sample comprising a rare-earth element, providing a covalent organic framework according to the present disclosure, and contacting the sample with the covalent organic framework under conditions effective to separate the rare-earth elements from the sample.

Another aspect of the present disclosure relates to method of making a covalent organic framework comprising repeating units of a moiety comprising Formula (I):

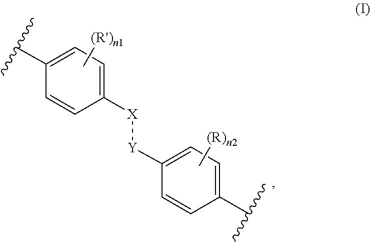

where
--- is a single or a double bond;
X is independently selected at each occurrence thereof from N or N(R³), wherein at least one X is N(R³);
Y is independently selected at each occurrence thereof from C(R¹)(R²) or C(R′);
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R¹ is H;
R² is optional and, if present, is —C(O)NHR⁴;
R³ is —C(O)R⁵;
R⁴ is independently selected at each occurrence thereof from the group consisting of cycloalkyl, —(CH₂)ₘC(O)OC₁₋₆ alkyl, and —(CH₂)ₘC(O)NH(CH₂)ₖNH₂, wherein —(CH₂)ₘC(O)NH(CH₂)ₖNH₂ can be optionally substituted 1 to 3 times with R⁷;
R⁵ is selected from the group consisting of C₁₋₁₂ alkyl, C₂₋₁₂ alkenyl, or C₂₋₁₂ alkynyl, wherein C₁₋₁₂ alkyl can be optionally substituted 1 to 3 times with R⁶;
R⁶ is independently selected at each occurrence thereof from N₃, —C(O)OC₁₋₆ alkyl, —C(O)NH(CH₂)ₖNH₂, wherein —C(O)NH(CH₂)ₖNH₂ can be optionally substituted 1 to 3 times with R⁷;
R⁷ is —(CH₂)ₖ₁NH₂ optionally substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5, or 6; and
k₇ is 1, 2, 3, 4, 5, or 6.

This method comprises providing a covalent organic framework comprising
repeating units of a moiety comprising Formula (II):

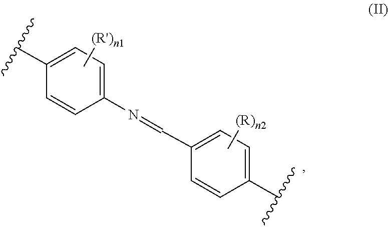

and
reacting the covalent organic framework comprising repeating units of a moiety comprising Formula (II) under conditions effective to produce the covalent organic framework.

Another aspect of the present disclosure relates to a method of making a modified covalent organic framework comprising repeating units of a moiety comprising Formula (I′a):

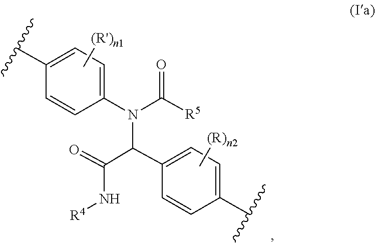

where
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R⁴ is —(CH₂)ₘC(O)NH(CH₂)ₖNH₂ substituted 1 to 3 times with R⁷;
R⁵ is C₁₋₁₂ alkyl substituted with R⁶;
R⁶ is —C(O)NH(CH₂)ₖNH₂ substituted 1 to 3 times with R⁷;
R⁷ is —(CH₂)ₖ₁NH₂ substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5, or 6; and
k₇ is 1, 2, 3, 4, 5, or 6.

This method comprises providing a covalent organic framework comprising repeating units of a moiety comprising Formula (I′b):

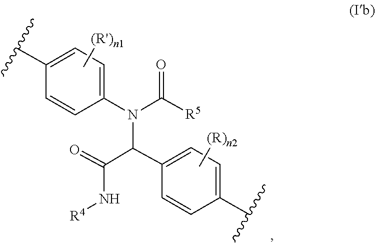

where R⁴ is —(CH₂)ₘC(O)NH(CH₂)ₖNH₂ substituted 1 to 3 times with —(CH₂)ₖ₁NH₂;
R⁵ is C₁₋₁₂ alkyl substituted with R⁶; and
R⁶ is —C(O)NH(CH₂)ₖNH₂ substituted 1 to 3 times with —(CH₂)ₖ₁NH₂; and reacting the covalent organic framework comprising repeating units of a moiety comprising Formula (I′b) under conditions effective to produce the modified covalent organic framework.

The library of imine-linked covalent organic frameworks (COFs) has grown significantly over the last two decades, featuring a variety of morphologies, pore sizes, and applications. An array of synthetic methods has been developed to expand the scope of the COF functionalities, however, most of these methods were designed to introduce functional scaffolds tailored to a specific application. Having a general approach to diversify COFs via late-stage incorporation of functional group handles would greatly facilitate the transformation of these materials into platforms for a variety of useful applications. The present disclosure provides a general strategy for introducing functional handles in COFs via the Ugi multicomponent reaction (FIG. 2). To demonstrate the versatility of this approach, two COFs with hexagonal and kagome morphologies were synthesized. Azide, alkyne, and vinyl functional groups where then introduced, which could be readily utilized for a variety of post-synthetic modifications. This facile approach enables the functionalization of any COFs containing imine linkages.

This facile post-synthetic modification (PSM) strategy based on the Ugi reaction can be used to introduce a variety of functional group handles into COFs with imine linkages. This approach radically simplifies the production of new functional COFs by converting any imine-based framework into a fully customizable platform. To demonstrate the versatility of this strategy, the Ugi multicomponent reaction was employed to install azide, alkyne, and alkene functional handles in two morphologically different COFs, the hexagonal TAPB-DMTP-COF (hex-COF) (Xu et al., “Stable, Crystalline, Porous, Covalent Organic Frameworks as a Platform for Chiral Organocatalysts,” Nat. Chem, 7(11):905-912 (2015), which is hereby incorporated by reference in its entirety) and kagome TAPS-DMTP-COF (kag-COF) (Lyle et al., “Multistep Solid-State Organic Synthesis of Carbamate-Linked Covalent Organic Frameworks,” J. Am. Chem. Soc., 141(28):11253-11258 (2019), which is hereby incorporated by reference in its entirety) (FIG. 3).

Rare-earth elements (REEs) are present in a broad range of critical materials. The development of solid adsorbents for REE capture could enable the cost-effective recycling of REE-containing magnets and electronics. In this context, COFs are promising candidates for REE adsorption due to their exceptionally high surface area. Despite having attractive physical properties, COFs are heavily underutilized for REE capture applications due to their limited lifecycle in aqueous acidic environments, as well as synthetic challenges associated with the incorporation of ligands suitable for REE capture. The present disclosure shows how the Ugi multicomponent reaction can be leveraged to post-synthetically modify imine-based COFs for the introduction of diglycolic acid (DGA) moiety, an efficient scaffold for REE capture (FIG. 4). The adsorption capacity of the DGA-functionalized COF was found to be more than 40 times higher than that of the pristine imine COF precursor and more than four times higher than that of the next-best reported DGA-functionalized solid support. This rationally designed COF has appealing characteristics of high adsorption capacity, fast and efficient capture and release of the REE ions, and reliable recyclability, making it one of the most promising adsorbents for solid-liquid REE ion extractions reported to date.

This novel and general PSM strategy utilizes a facile Ugi multi-component reaction to introduce a number of useful functional groups into any imine-based COFs while retaining their structural integrity and improving chemical stability (Volkov et al., “General Strategy for Incorporation of Functional Group Handles into Covalent Organic Frameworks via the Ugi Reaction,” J. Am. Chem. Soc., 145(11):6230-6239 (2023), which is hereby incorporated by reference in its entirety). This Ugi PSM strategy can be leveraged to rapidly tailor molecular functionality and improve the chemical integrity of an imine-based COF, both essential for producing recyclable material for efficient capture and release of REE ions. To incorporate a functional ligand into the COF, a well-established diglycolic acid (DGA) moiety known for its efficient coordination of REE ions was selected (Ogata et al., “Adsorption Mechanism of Rare Earth Elements by Adsorbents with Diglycolamic Acid Ligands,” Hydrometallurgy, 163:156-160 (2016); Shinozaki et al., “Preparation of Polymeric Adsorbents Bearing Diglycolamic Acid Ligands for Rare Earth Elements,” Ind. Eng. Chem. Res., 57(33):11424-11430 (2018); Ibrahim et al., “Selective Extraction of Light Lanthanides(III) by N,N-Di(2-ethylhexyl)-diglycolamic Acid: A Comparative Study with N,N-Dimethyl-diglycolamic Acid as a Chelator in Aqueous Solutions,” ACS Omega, 4:20797-20806 (2019), which are hereby incorporated by reference in their entirety).

### BRIEF DESCRIPTION OF THE DRAWINGS

FIG. 1 shows imine bond transformations in imine-linked covalent organic frameworks (COFs).

FIG. 2 shows both the previous approach to installing functional group handles in COFs and the strategy disclosed herein.

FIG. 3 illustrates the Ugi multi-component reaction in COFs.

FIG. 4 illustrates a strategy for capturing rare earth elements from aqueous solution using Ugi-COFs as described herein.

FIG. 5 shows ¹H→¹³C CP saturation recovery curves normalized intensity buildup of hex-COF and hex-Ugi-COF-OAc-[1.0].

FIG. 6 shows the ¹H solution-state NMR spectrum of the model compound.

FIG. 7 shows the ¹³C solution-state NMR spectrum of the model compound.

FIGS. 8A-8E provide characterization data for Ugi adducts. FIG. 8A shows Fourier-transform infrared (FTIR) spectra of as-synthesized hex-COF (top), hex-Ugi-COF—OAc-[1.0](middle), and hex-Ugi-COF-OAc-[1.5](bottom). FIG. 8B shows experimental powder X-ray diffraction (PXRD) patterns of as-synthesized hex-COF, hex-Ugi-COF—OAc-[1.0], and hex-Ugi-COF-OAc-[1.5]. The FIG. 8B insets show scanning electron microscopy (SEM) images of as-synthesized hex-COF (top) and hex-Ugi-COF-OAc-[1.0](middle), and juxtaposed PXRD patterns, showing subtle higher angle shifts (bottom). FIG. 8C shows XPS spectra of as-synthesized hex-COF (top), hex-Ugi-COF-OAc-[1.0](middle), and hex-Ugi-COF-OAc-[1.5](bottom). FIG. 8D shows dynamic nuclear polarization (DNP)-enhanced ¹⁵N cross-polarization MAS (CPMAS) solid-state NMR spectra of as-synthesized hex-COF, and hex-Ugi-COF-OAc-[1.0]. FIG. 8E shows ¹³C solution-state NMR of the model compound, ¹³C CPMAS solid-state NMR spectra of as-synthesized hex-COF, and hex-Ugi-COF-OAc-[1.0].

FIGS. 9A-9F provide characterization data for Ugi adducts. FIG. 9A shows FTIR spectra of as-synthesized kag-COF (top) and kag-Ugi-COF-OAc-[1.0](bottom). FIG. 9B shows experimental PXRD patterns of as-synthesized kag-COF (top) and kag-Ugi-COF-OAc-[1.0](bottom). The FIG. 9B insets show SEM images of as-synthesized kag-COF (top) and kag-Ugi-COF-OAc-[1.0](bottom). FIG. 9C shows XPS spectra of as-synthesized kag-COF (top) and kag-Ugi-COF-OAc-[1.0](bottom). FIG. 9D shows FTIR spectra of azide (top), alkyne (middle), and vinyl (bottom) derivatives of the kag-Ugi-COF. FIG. 9E shows experimental PXRD patterns of azide (top), alkyne (middle), and vinyl (bottom) derivatives of the kag-Ugi-COF. FIG. 9F shows ¹³C solution-state NMR of the model compound (top), ¹³C CPMAS solid-state NMR spectra of as-synthesized kag-COF (middle), and kag-Ugi-COF-OAc-[1.0](bottom).

FIGS. 10A-10B show FTIR spectra (FIG. 10A) and experimental PXRD patterns (FIG. 10B) of azide, alkyne, and vinyl derivatives of the hex-Ugi-COF.

FIGS. 11A-11B show PXRD patterns of hex-COF and hex-Ugi-COF-OAc-[1.0](FIG. 11A), and kag-COF and kag-Ugi-COF-OAc-[1.0](FIG. 11B) after stability tests. For the stability testing, each material (10 mg) was added to a 1-dram glass vial, suspended in 12 M HCl (0.5 mL) or 12 M KOH (0.5 mL), and heated to 80° C. for 24 hours. Solids were then isolated by filtration, washed with water, acetone, and dried at 100° C. under reduced pressure for 24 hours. The dried powders were then analyzed by PXRD to assess the ordering of the framework.

FIG. 12 shows the N₂ adsorption isotherm of as-synthesized hex-COF (top), hex-Ugi-COF-OAc-[1.0](middle), and hex-Ugi-COF-OAc-[1.5](bottom). Closed and open circles represent adsorption and desorption points, respectively.

FIG. 13 shows the N₂ adsorption isotherm of as-synthesized kag-COF and kag-Ugi-COF-OAc-[1.0]. Closed and open circles represent adsorption and desorption points, respectively.

FIG. 14 shows the N₂ adsorption isotherm of hex-Ugi-COF—C≡C-[1.5]. Closed and open circles represent adsorption and desorption points, respectively.

FIG. 15 shows the N₂ adsorption isotherm of kag-Ugi-COF—C≡C-[1.0]. Closed and open circles represent adsorption and desorption points, respectively.

FIG. 16 shows ¹³C CPMAS solid-state NMR spectra of (upper) hex-COF and (lower) hex-Ugi-COF—OAC-[1.0] impregnated with a 16 mM TEKPol TCE solution. The spectra were obtained with and without microwaves. The DNP enhancement for the aromatic carbon signals is indicated. The intensity of the NMR spectrum obtained without DNP was scaled by the indicated value of e_{C CP}.

FIG. 17 shows the ¹³C CPMAS solid-state NMR spectrum of hex-Ugi-COF—C≡C-[1.5].

FIG. 18 shows the ¹³C CPMAS solid-state NMR spectrum of kag-Ugi-COF—C≡C-[1.0].

FIG. 19 shows thermogravimetric analysis (TGA) curves of hex-COF and hex-Ugi-COF-OAc-[1.0].

FIG. 20 shows TGA curves of kag-COF and kag-Ugi-COF-OAc-[1.0].

FIG. 21 shows evolution of the Ugi-COF structural designs for the effective and recyclable adsorption of the REE elements.

FIG. 22A-22D show the synthetic route (FIG. 22A), FTIR spectra (FIG. 22B), ¹³C CPMAS solid-state nuclear magnetic resonance (SSNMR) spectra (FIG. 22C), and N₂ adsorption isotherms (FIG. 22D) (closed and open circles represent adsorption and desorption points, respectively) of as-synthesized imine-COF, Ugi-COF, Ugi-COF—NH₂, and Ugi-COF-DGA.

FIG. 23 shows experimental PXRD patterns of ordered, as-synthesized imine-COF, and Ugi-COF, Ugi-COF—NH₂, and Ugi-COF-DGA with no apparent peaks.

FIGS. 24A-24B show atomic force microscopy (AFM) images and their corresponding cross-sectional height profiles of as-synthesized imine-COF (FIG. 24A) and Ugi-COF-DGA (FIG. 24B).

FIG. 25 shows TGA curves of imine-COF, Ugi-COF, Ugi-COF—NH₂, and Ugi-COF-DGA.

FIG. 26A shows the Nd adsorption capacity of as-synthesized imine-COF, Ugi-COF—NH₂, and Ugi-COF-DGA. FIG. 26B shows a pH-dependent study of the Nd adsorption capacity of the Ugi-COF-DGA. FIG. 26C shows an isotherm plot of the adsorption capacity of the Ugi-COF-DGA at different initial Nd concentrations. FIG. 26D shows adsorption and desorption performance of the Ugi-COF-DGA over three recycles. FIG. 26E shows a time-dependent study of the adsorption performance of the Ugi-COF-DGA. FIG. 26F shows a comparison of the maximum adsorption capacities for the Ugi-COF-DGA and other DGA-functionalized solid supports. Unless otherwise stated, adsorption capacities of as-synthesized materials were tested using 10 mg of adsorbent in 50 mL of 50 ppm Nd solution at 25° C. for 20 hours. Desorption was carried out by suspending Nd-adsorbed material in 10 mL of 2.0 M HNO₃ at 25° C. for 20 hours.

FIG. 27 provides zeta potential of the Ugi-COF-DGA for different concentrations of the Nd solutions.

FIG. 28 shows Langmuir model of the adsorption isotherm for the Ugi-COF-DGA.

FIG. 29 shows ¹³C CPMAS SSNMR spectra of as-synthesized Ugi-COF-DGA (top) and Ugi-COF-DGA after five recycling runs (bottom).

FIGS. 30A-30B show pseudo-first (FIG. 30A) and pseudo-second-order (FIG. 30B) fittings of the kinetics model for the Ugi-COF-DGA.

FIG. 31 shows a time-dependent study of the desorption performance of the Ugi-COF-DGA.

FIG. 32 shows a comparative time-dependent study of the adsorption performance of the Ugi-COF-DGA performance using Nd and Dy ions. Dashed lines are used as a guide for the eye.

FIG. 33 shows the adsorption capacities of Nd and Dy.

FIG. 34 shows FTIR spectra of Ugi-COF-DGA and Nd [symbol] Ugi-COF-DGA.

[symbol]

FIG. 35 shows XPS Nd 4d spectra of Nd(NO₃)₃ and Nd [symbol] Ugi-COF-DGA.

FIG. 36 shows EDS mapping of Nd [symbol] Ugi-COF-DGA: (left) N and Nd EDS maps overlapped with STEM image; (center) Nd EDS image; (right) N EDS image.

FIG. 37 shows the EDS spectrum of the Nd [symbol] Ugi-COF-DGA.

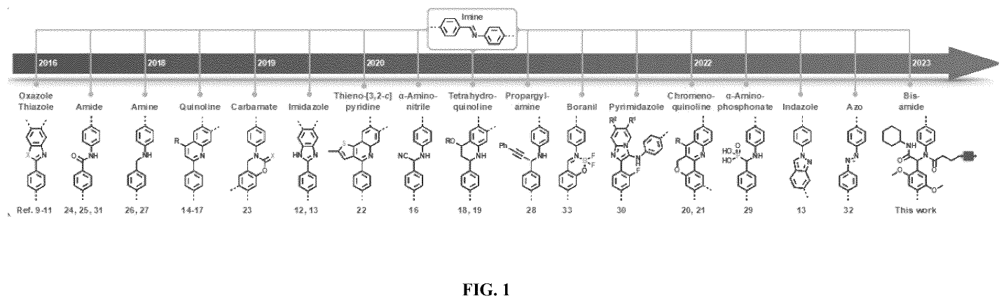

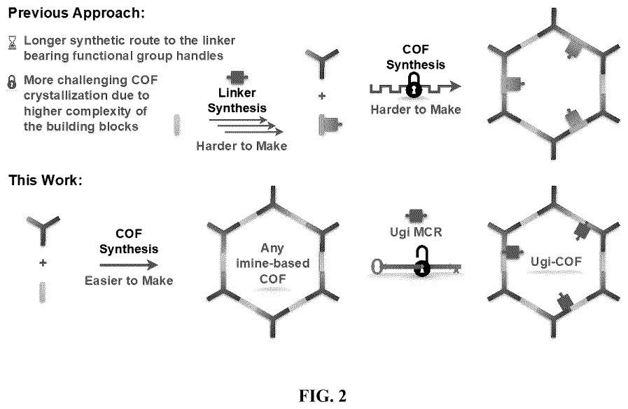

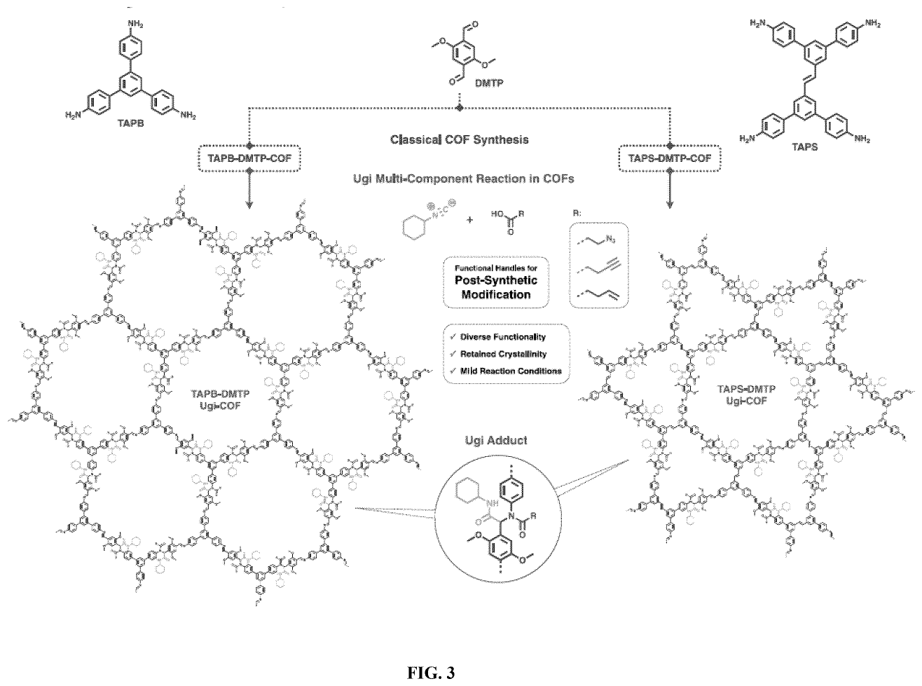

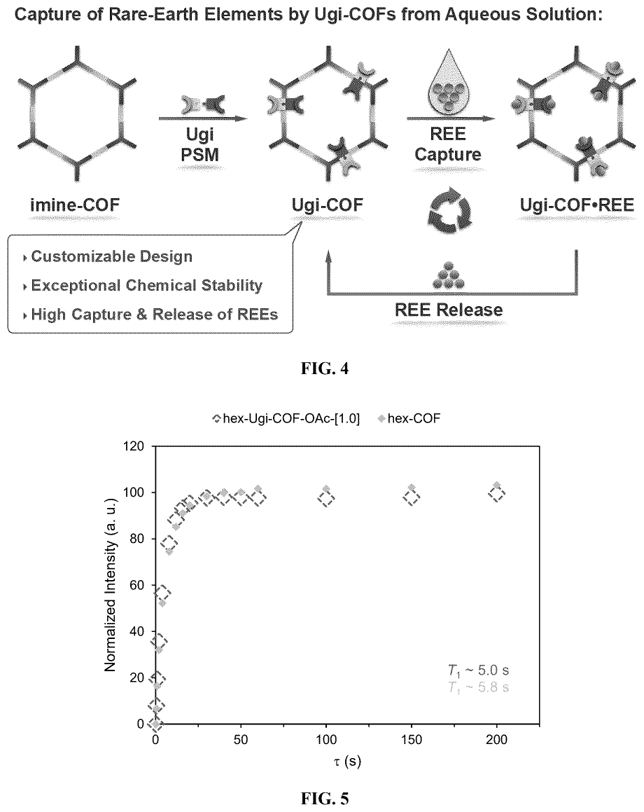

### DETAILED DESCRIPTION

One aspect of the present disclosure relates to a covalent organic framework comprising repeating units of a moiety comprising Formula (I):

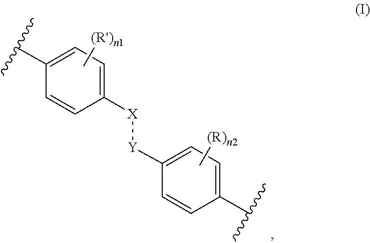

where
--- is a single or a double bond;
X is independently selected at each occurrence thereof from N or N(R³), wherein at least one X is N(R³);
Y is independently selected at each occurrence thereof from C(R¹)(R²) or CR);
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R¹ is H;
R² is optional and, if present, is —C(O)NHR⁴;
R³ is —C(O)R⁵;
R⁴ is independently selected at each occurrence thereof from the group consisting of cycloalkyl, —(CH₂)ₘC(O)OC₁₋₆ alkyl, and —(CH₂)ₘC(O)NH(CH₂)ₖNH₂, wherein —(CH₂)ₘC(O)NH(CH₂)ₖNH₂ can be optionally substituted 1 to 3 times with R⁷;
R⁵ is selected from the group consisting of C₁₋₁₂ alkyl, C₂₋₁₂ alkenyl, or C₂₋₁₂ alkynyl, wherein C₁₋₁₂ alkyl can be optionally substituted 1 to 3 times with R⁶;
R⁶ is independently selected at each occurrence thereof from N₃, —C(O)OC₁₋₆ alkyl, —C(O)NH(CH₂)ₖNH₂, wherein —C(O)NH(CH₂)ₖNH₂ can be optionally substituted 1 to 3 times with R⁷;
R⁷ is —(CH₂)ₖ₁NH₂ optionally substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5, or 6; and
k₇ is 1, 2, 3, 4, 5, or 6.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

In this specification and the appended claims, the singular forms “a,” “an,” and “the” include plural references unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements, or method steps.

The terms “comprising,” “comprises,” and “comprised of” also encompass the term “consisting of” The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of”

Terms of degree such as “substantially,” “about,” and “approximately” and the symbol “˜” as used herein mean a reasonable amount of deviation of the modified term such that the end result is not significantly changed. These terms of degree should be construed as including a deviation of at least ±0.1% (and up to ±1%, ±5%, or ±10%) of the modified term if this deviation would not negate the meaning of the word it modifies. Unless otherwise clear from context, all numerical values provided herein are modified by the term about. All numerical values provided herein that are modified by terms of degree set forth in this paragraph (e.g., “substantially,” “about,” “approximately,” and “˜”) are also explicitly disclosed without the term of degree. For example, “about 1%” is also explicitly disclosed as “10”.

The term “and/or” as used herein means that the listed items are present, or used, individually or in combination. In effect, this term means that “at least one of” or “one or more” of the listed items is used or present.

The recitation of numerical ranges by endpoints includes all numbers and fractions subsumed within the respective ranges, as well as the recited endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

The term “alkyl” means an aliphatic hydrocarbon group which may be straight or branched having about 1 to about 12 carbon atoms in the chain. For example, straight or branched carbon chain could have 1 to 6 carbon atoms. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “alkenyl” means an aliphatic hydrocarbon group containing a carbon-carbon double bond and which may be straight or branched having about 2 to about 12 carbon atoms in the chain. Particular alkenyl groups have 2 to about 6 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. The term “alkenyl” may also refer to a hydrocarbon chain having 2 to 6 carbons containing at least one double bond and at least one triple bond.

The term “alkynyl” means an aliphatic hydrocarbon group containing a carbon-carbon triple bond and which may be straight or branched having about 2 to about 12 carbon atoms in the chain. Particular alkynyl groups have 2 to about 6 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl.

The term “cycloalkyl” means a non-aromatic mono- or multicyclic ring system of about 3 to about 12 carbon atoms, preferably of about 3 to about 8 carbon atoms. Exemplary monocyclic cycloalkyls include cyclopentyl, cyclohexyl, cycloheptyl, bicyclo[1.1.1]pentyl, and the like.

The term “heteroaryl” means an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “Heteroaryl”. Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like. The term “heteroarylene” refers to a group obtained by removal of a hydrogen atom from a heteroaryl group. Exemplary heteroarylene groups include, but are not limited to, groups derived from the heteroaryl groups described above.

The term “monocyclic” used herein indicates a molecular structure having one ring.

The term “polycyclic” or “multicyclic” used herein indicates a molecular structure having two or more rings, including, but not limited to, fused, bridged, or spiro rings.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

In some embodiments of the covalent organic framework, m is 1, k is 2, k₁ is 2, k₂ is 1, and k₃ is 1.

In some embodiments, R′ is H.

According to the present disclosure, X in the covalent organic framework comprising repeating units of a moiety comprising Formula (I) is independently selected at each occurrence thereof from N or N(R³), wherein at least one X is N(R³). In some embodiments, X is N(R³).

According to the present disclosure, from about 1% to about 100% of X in the covalent organic framework comprising repeating units of a moiety comprising Formula (I) is NR³. In some embodiments, from about 1% to about 10%, from about 1% to about 20%, from about 1% to about 30%, from about 1% to about 40%, from about 1% to about 50%, from about 1% to about 60%, from about 1% to about 70%, from about 1% to about 80%, from about 1% to about 90%, from about 10% to about 20%, from about 10% to about 30%, from about 10% to about 40%, from about 10% to about 50%, from about 10% to about 60%, from about 10% to about 70%, from about 10% to about 80%, from about 10% to about 90%, from about 10% to about 100%, from about 20% to about 30%, from about 20% to about 40%, from about 20% to about 50%, from about 20% to about 60%, from about 20% to about 70%, from about 20% to about 80%, from about 20% to about 90%, from about 20% to about 100%, from about 30% to about 40%, from about 30% to about 50%, from about 30% to about 60%, from about 30% to about 70%, from about 30% to about 80%, from about 30% to about 90%, from about 30% to about 100%, from about 40% to about 50%, from about 40% to about 60%, from about 40% to about 70%, from about 40% to about 80%, from about 40% to about 90%, from about 40% to about 100%, from about 50% to about 60%, from about 50% to about 70%, from about 50% to about 80%, from about 50% to about 90%, from about 50% to about 100%, from about 60% to about 70%, from about 60% to about 80%, a from about 60% to about 90%, from about 60% to about 100%, %, from about 70% to about 80%, from about 70% to about 90%, from about 70% to about 100%, from about 80% to about 90%, from about 80% to about 100%, or from about 90% to about 100% of X in the covalent organic framework comprising repeating units of a moiety comprising Formula (I) is NR³.

In some embodiments, the repeating units of a moiety comprise Formula (I′):

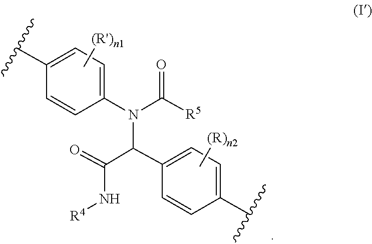

In some embodiments, the repeating units of a moiety comprise a structure selected from the group consisting of:

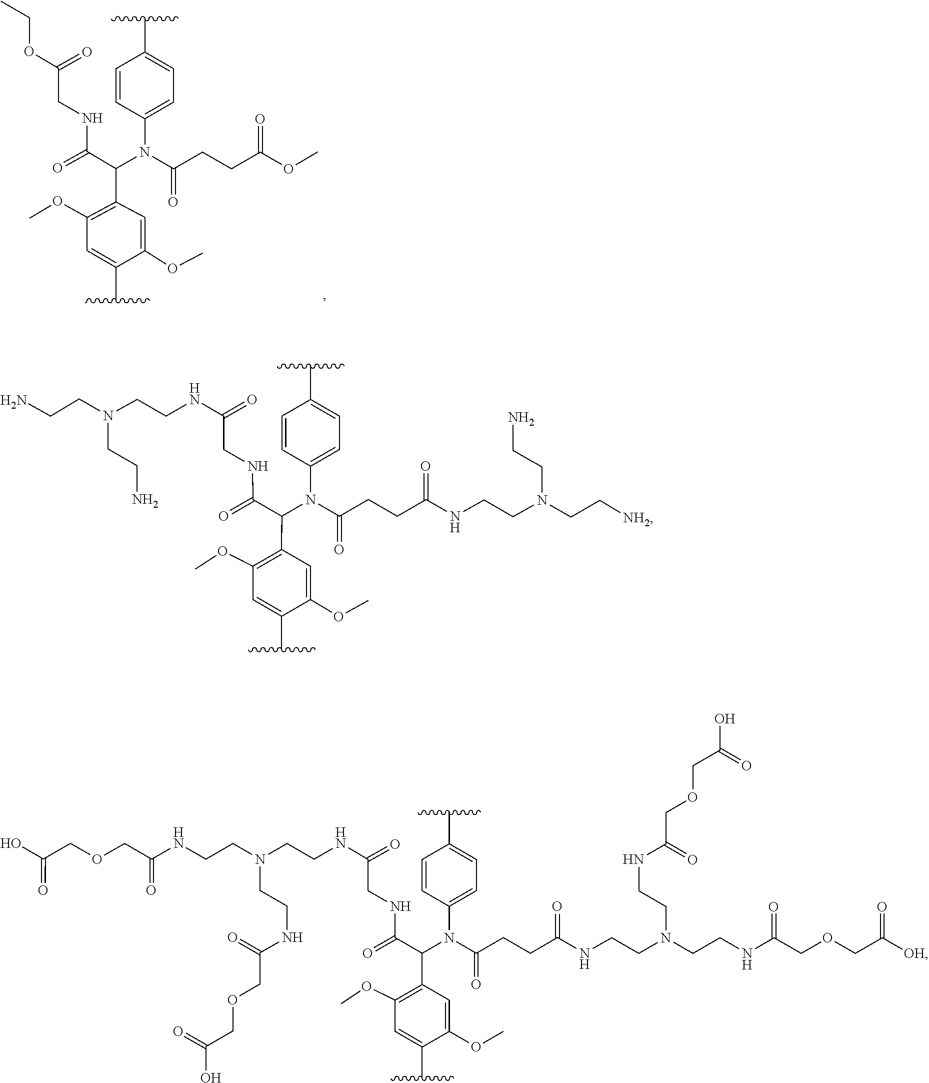

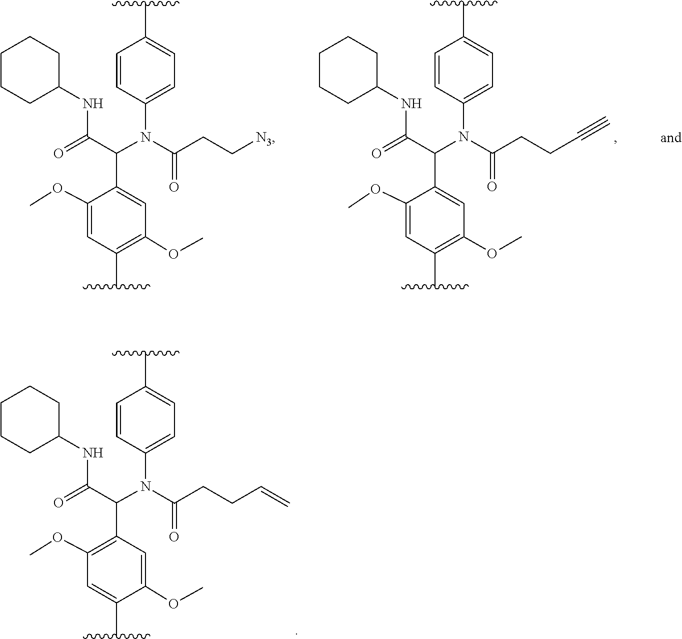

In some embodiments, the repeating units of a moiety comprise a structure selected from the group consisting of:

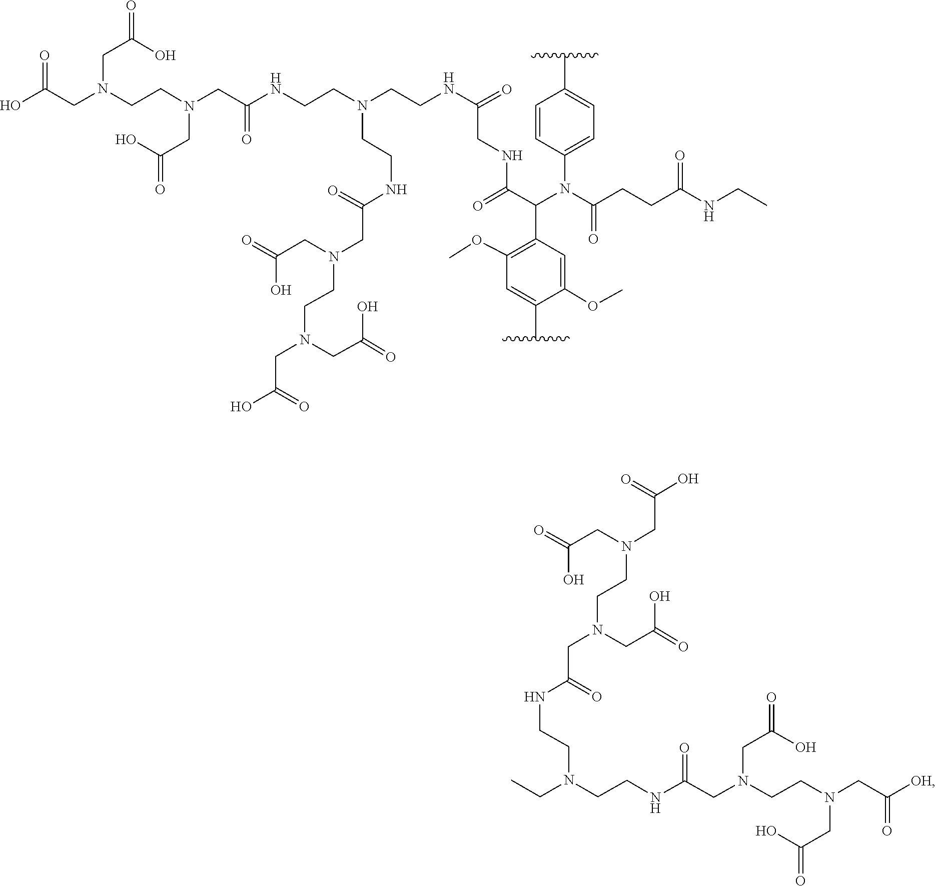

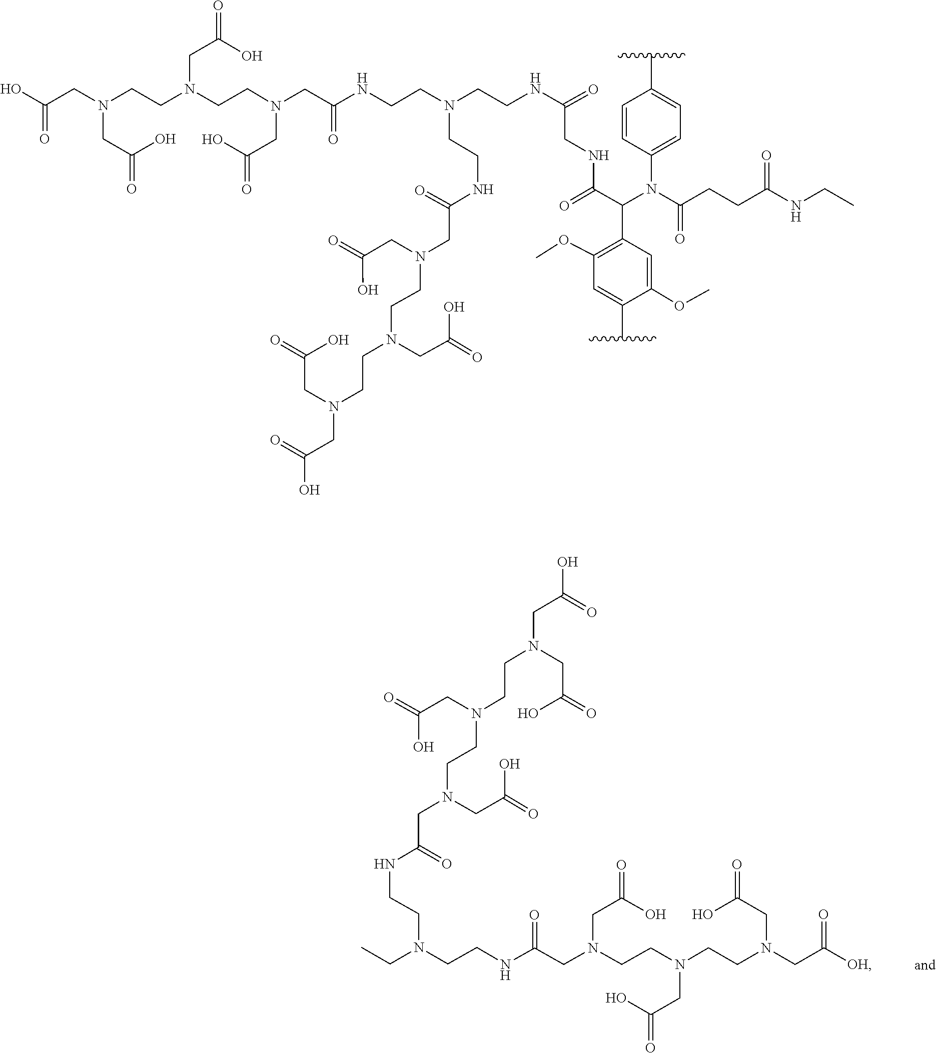

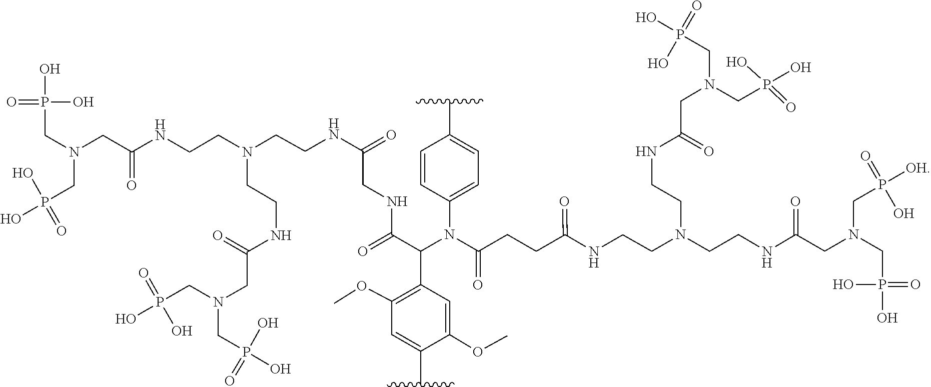

In some embodiments, the repeating units of a moiety have a structure of Formula (Ia):

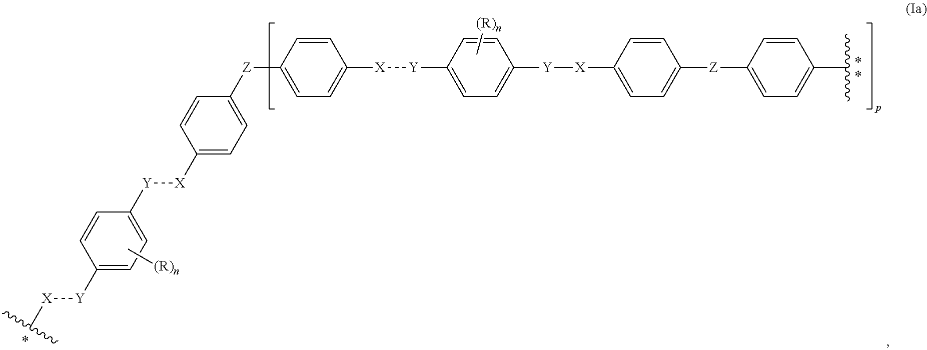

where

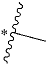

is the point of attachment of the moiety of Formula (Ia) to

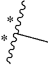

of another moiety of Formula (Ia);
Z is selected from the group consisting of C, CH,

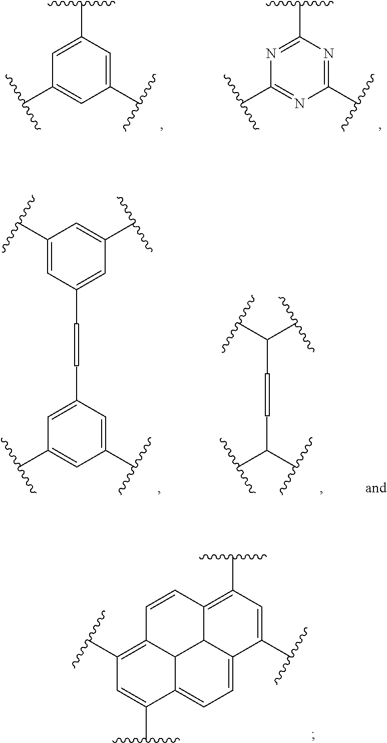

and
p is 2 or 3.

In some embodiments, the repeating units of a moiety have a structure of Formula (Ib):

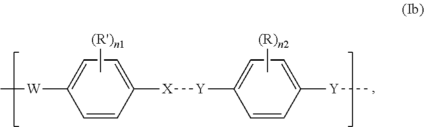

where
W is selected from the group consisting of X,

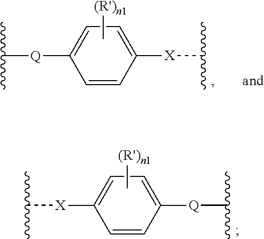

and
Q is optional, and if present, is —NHC(O)— or —C(O)NH—.

In some embodiments, the covalent organic framework comprises a moiety of Formula (Ic):

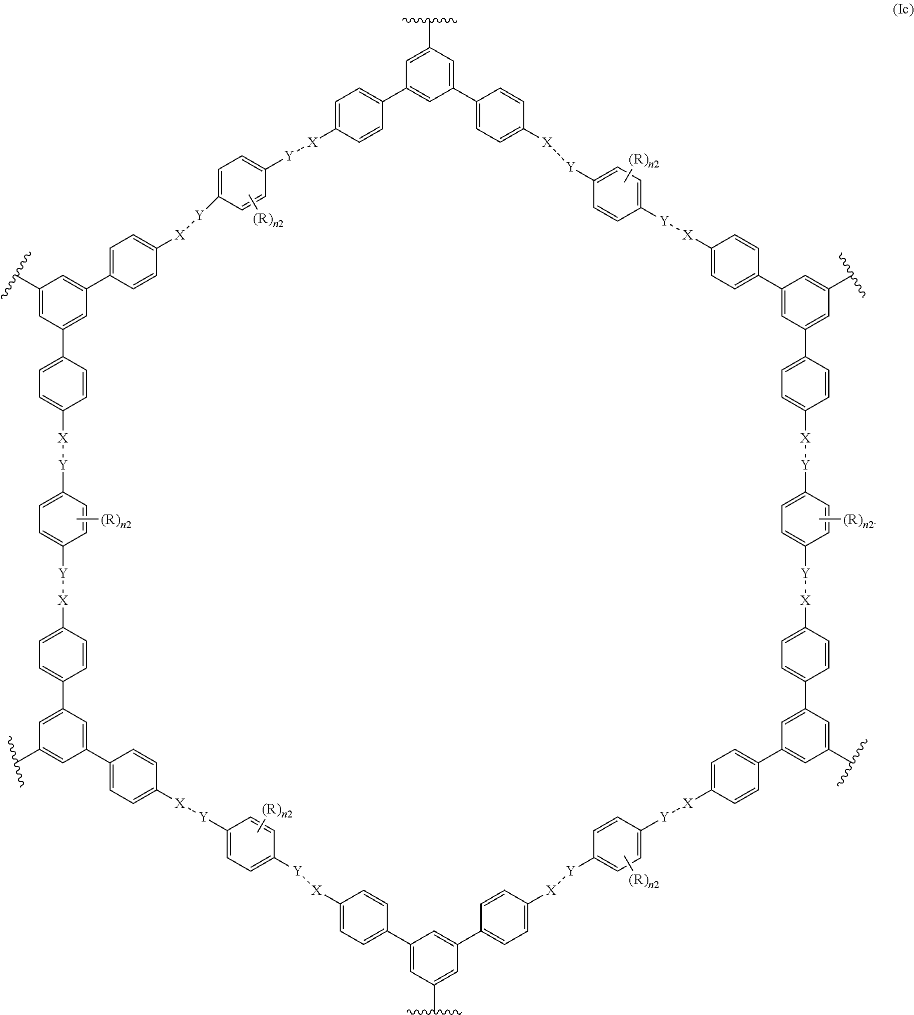

Another aspect of the present disclosure relates to method of separating rare-earth elements. This method comprises providing a sample comprising a rare-earth element, providing a covalent organic framework according to the present disclosure, and contacting the sample with the covalent organic framework under conditions effective to separate the rare-earth elements from the sample.

Suitable rare earth elements that can be present in the sample include La, Ce, Pr, Nd, Pm, Sm, Eu, Gd, Tb, Dy, Ho, Er, Tm, Yb, Lu, Y, and Sc. In some embodiments, the sample contains Nd, La, Lu, Pr, or Dy salts, such as nitrates, chlorides, and/or acetates. In some embodiments, the sample contains Dy(NO₃)₃ or Nd(NO₃)₃. In one embodiment, the sample contains Nd(NO₃).

In some embodiments, the sample contains two, three, four, or more rare-earth elements. In some embodiments, the sample contains two or more rare-earth elements. In some embodiments, the sample contains Nd(NO₃)₃ and Dy(NO₃)₃.

In some embodiments, contacting the sample with the covalent organic framework results in the capture (adsorption) of the rare earth element inside the covalent organic framework. In some embodiments, when the sample contains two or more rare-earth elements, contacting the sample with the covalent organic framework results in more effective capture (adsorption) of one rare-earth element over another rare-earth element. In some embodiments one rare-earth element is selectively captured in the presence of another rare-earth element. For example, one rare-earth element can be selectively captured in the presence of another rare-earth element in a ratio of about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1.

In some embodiments Dy ions are captured more effectively than Nd ions. For example, in some embodiments, Dy ions are selectively captured in the presence of Nd ions in a ratio of about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1.

In some embodiments, the method further comprises isolating the covalent organic framework containing the rare-earth elements from the sample. In some embodiments, such isolation is carried out by filtration.

In some embodiments, the method further comprises recovering the rare-earth element from the covalent organic framework.

In some embodiments, the recovering is carried out by suspending the covalent organic framework containing the rare-earth elements in an inorganic acid, such as nitric acid, at about 25° C. for about 20 hours and filtering the covalent organic framework. For example, in some embodiments, the covalent organic framework containing the rare-earth elements can be suspended in an inorganic acid, such as nitric acid, at about 20° C., about 21° C., about 22° C., about 23° C., about 24° C., about 25° C., about 26° C., about 27° C., about 28° C., about 29° C., or about 30° C. for about 15 hours, about 16 hours, about 17 hours, about 18 hours, about 19 hours, about 20 hours, about 21 hours, about 22 hours, about 23 hours, about 24 hours, or about 25 hours and then filtered.

The general scheme for the covalent organic framework of the present disclosure is shown in Scheme 1.

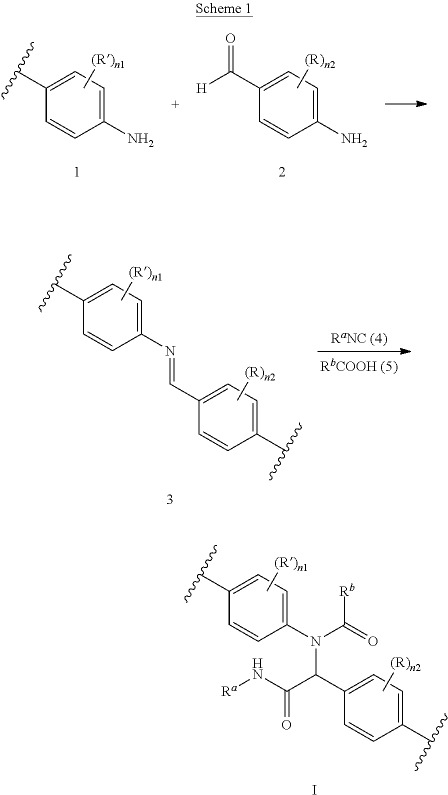

Reaction between an amine comprising a moiety having a Formula 1 and an aldehyde comprising a moiety having a Formula 2 leads to formation of a covalent organic framework comprising repeating units of a moiety comprising Formula 3. The reaction can be carried out in a variety of solvents, for example in methanol, ethanol, acetonitrile, water, toluene, mesitylene, methylene chloride (CH₂Cl₂), tetrahydrofuran (THF), dimethylformamide (DMF), or other such solvents or in the mixture of such solvents. This reaction can be carried out at room temperature or at elevated temperatures. Ugi reaction between the covalent organic framework 3, isocyanate (4), and carboxylic acid (5) leads to formation of the covalent organic framework comprising repeating units of a moiety comprising Formula (I). The reaction can be carried out in a variety of solvents, for example in water, methanol, ethanol, acetonitrile, methylene chloride (CH₂Cl₂), tetrahydrofuran (THF), dimethylformamide (DMF), or other such solvents or in the mixture of such solvents. This reaction can be carried out at room temperature or at elevated temperatures.

The general scheme for the modified covalent organic framework of the present disclosure is shown in Scheme 2.

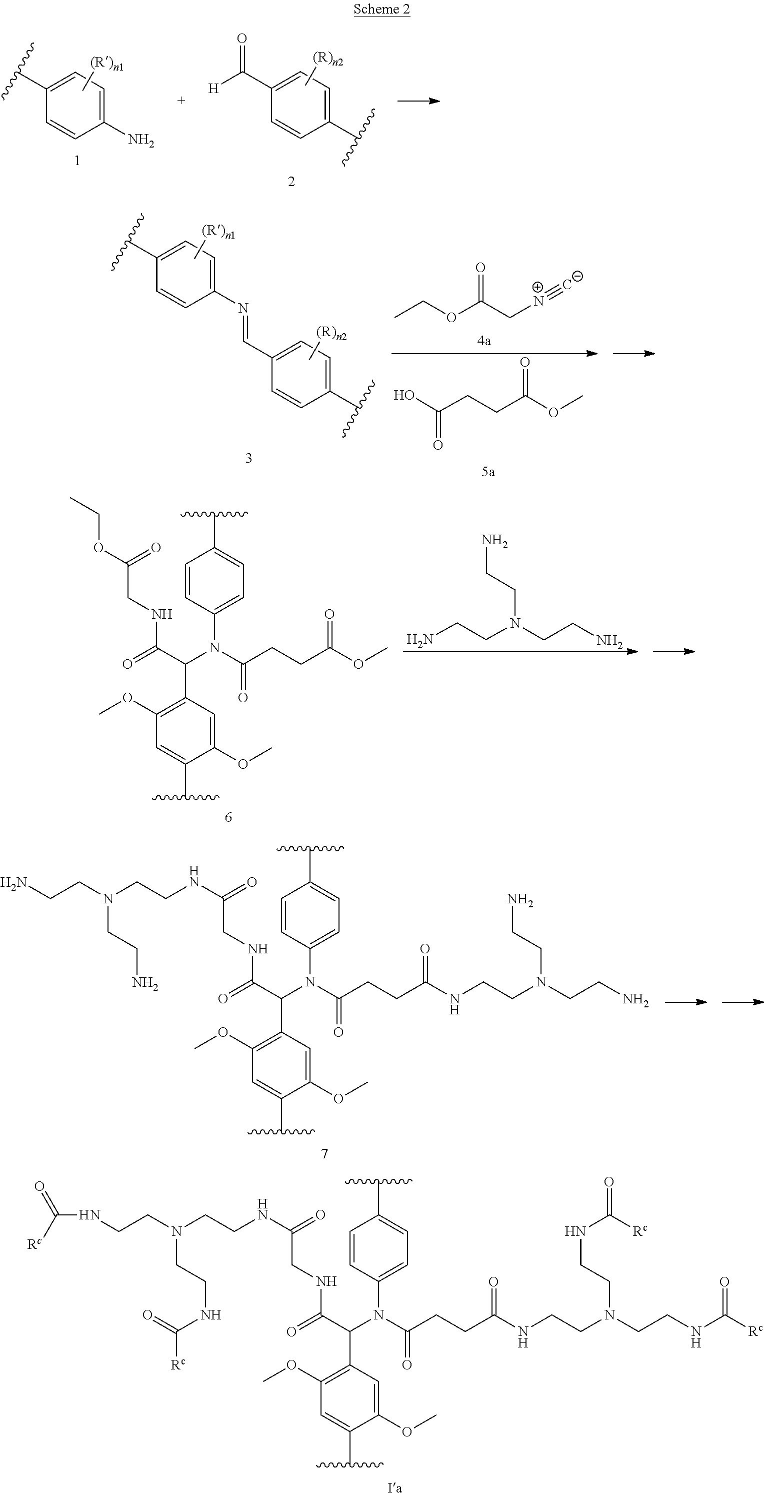

Reaction between an amine comprising a moiety having a Formula 1 and an aldehyde comprising a moiety having a Formula 2 leads to formation of a covalent organic framework comprising repeating units of a moiety comprising Formula 3. The reaction can be carried out in a variety of solvents, for example in methanol, ethanol, acetonitrile, water, toluene, mesitylene, methylene chloride (CH₂Cl₂), tetrahydrofuran (THF), dimethylformamide (DMF), or other such solvents or in the mixture of such solvents. This reaction can be carried out at room temperature or at elevated temperatures. Ugi reaction between the covalent organic framework 3, an isocyanate (4a), and a carboxylic acid (5a) leads to formation of a covalent organic framework comprising repeating units of a moiety comprising Formula (6). The reaction can be carried out in a variety of solvents, for example in water, methanol, ethanol, acetonitrile, methylene chloride (CH₂Cl₂), tetrahydrofuran (THF), dimethylformamide (DMF), or other such solvents or in the mixture of such solvents. This reaction can be carried out at room temperature or at elevated temperatures. Amidation of the covalent organic framework (6) leads to formation of a covalent organic framework comprising repeating units of a moiety comprising Formula (7). The reaction can be carried out in a variety of solvents, for example in methanol, water, methylene chloride (CH₂Cl₂), tetrahydrofuran (THF), dimethylformamide (DMF), or other such solvents or in the mixture of such solvents. This reaction can be carried out room temperature or at elevated temperatures. Acylation of the covalent organic framework (7) leads to formation of the covalent organic framework comprising repeating units of a moiety comprising Formula (Ia). Any suitable acylating agent can be used. The reaction can be carried out in a variety of solvents, for example in methanol, water, methylene chloride (CH₂Cl₂), tetrahydrofuran (THF), dimethylformamide (DMF), or other such solvents or in the mixture of such solvents. This reaction can be carried out room temperature or at elevated temperatures.

Another aspect of the present disclosure relates to method of making a covalent organic framework comprising repeating units of a moiety comprising Formula (I):

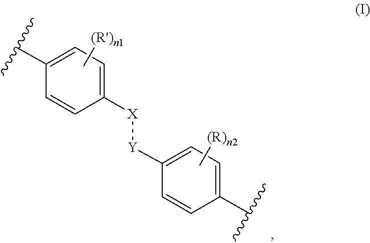

where
--- is a single or a double bond;
X is independently selected at each occurrence thereof from N or N(R³), wherein at least one X is N(R³);
Y is independently selected at each occurrence thereof from C(R¹)(R²) or C(R¹);
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R¹ is H;
R² is optional and, if present, is —C(O)NHR⁴;
R³ is —C(O)R⁵;
R⁴ is independently selected at each occurrence thereof from the group consisting of cycloalkyl, —(CH₂)ₘC(O)OC₁₋₆ alkyl, and —(CH₂)ₘC(O)NH(CH₂)ₖNH₂, wherein —(CH₂)ₘC(O)NH(CH₂)ₖNH₂ can be optionally substituted 1 to 3 times with R⁷;
R⁵ is selected from the group consisting of C₁₋₁₂ alkyl, C₂₋₁₂ alkenyl, or C₂₋₁₂ alkynyl, wherein C₁₋₁₂ alkyl can be optionally substituted 1 to 3 times with R⁶;
R⁶ is independently selected at each occurrence thereof from N₃, —C(O)OC₁₋₆ alkyl, —C(O)NH(CH₂)ₖNH₂, wherein —C(O)NH(CH₂)ₖNH₂ can be optionally substituted 1 to 3 times with R⁷;
R⁷ is —(CH₂)ₖ₁NH₂ optionally substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5,or 6; and
k₇ is 1, 2, 3, 4, 5, or 6.

This method comprises providing a covalent organic framework comprising repeating units of a moiety comprising Formula (II):

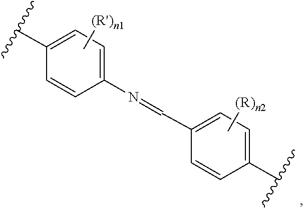

and
reacting the covalent organic framework comprising repeating units of a moiety comprising Formula (II) under conditions effective to produce the covalent organic framework.

In some embodiments, the reacting comprises providing a compound of Formula (III):

R⁴—N≡C (III);

providing a compound of Formula (IV):

R⁵COOH (IV); and
reacting the covalent organic framework comprising repeating units of a moiety comprising Formula (II) with the compound of Formula (III) and the compound of Formula (IV) to produce the covalent organic framework.

In some embodiments, the reacting the covalent organic framework comprising repeating units of a moiety comprising Formula (II) with the compound of Formula (III) and the compound of Formula (IV) is carried out at a temperature from about 20° C. to about 200° C. For example, in some embodiments, the reacting of the covalent organic framework comprising repeating units of a moiety comprising Formula (II) with the compound of Formula (III) and the compound of Formula (IV) is carried out at a temperature from about 20° C. to about 40° C., about 20° C. to about 60° C., about 20° C. to about 80° C., about 20° C. to about 100° C., about 20° C. to about 120° C., about 20° C. to about 140° C., about 20° C. to about 160° C., or about 20° C. to about 180° C., about 30° C. to about 50° C., about 35° C. to about 45° C., or about 40° C. to about 50° C.

In some embodiments, the reacting the covalent organic framework comprising repeating units of a moiety comprising Formula (II) with the compound of Formula (III) and the compound of Formula (IV) is carried out for at least 8 hours without stirring. For example, in some embodiments, the reacting is carried out for at least 8 hours, at least 10 hours, at least 12 hours, at least 14 hours, at least 16 hours, or at least 24 hours.

In some embodiments, the compound of Formula (III) is

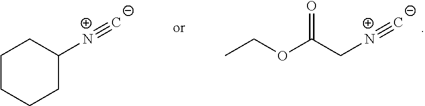

According to the present disclosure, the reaction between the covalent organic framework comprising repeating units of a moiety comprising Formula (II), the compound of Formula (III), and the compound of Formula (IV) results in the formation of the covalent organic framework comprising repeating units of a moiety comprising Formula (I) that contains from about 1% to about 100% of X being NR³. In some embodiments, from about 1% to about 10%, from about 1% to about 20%, from about 1% to about 30%, from about 1% to about 40%, from about 1% to about 50%, from about 1% to about 60%, from about 1% to about 70%, from about 1% to about 80%, from about 1% to about 90%, from about 10% to about 20%, from about 10% to about 30%, from about 10% to about 40%, from about 10% to about 50%, from about 10% to about 60%, from about 10% to about 70%, from about 10% to about 80%, from about 10% to about 90%, from about 10% to about 100%, from about 20% to about 30%, from about 20% to about 40%, from about 20% to about 50%, from about 20% to about 60%, from about 20% to about 70%, from about 20% to about 80%, from about 20% to about 90%, from about 20% to about 100%, from about 30% to about 40%, from about 30% to about 50%, from about 30% to about 60%, from about 30% to about 70%, from about 30% to about 80%, from about 30% to about 90%, from about 30% to about 100%, from about 40% to about 50%, from about 40% to about 60%, from about 40% to about 70%, from about 40% to about 80%, from about 40% to about 90%, from about 40% to about 100%, from about 50% to about 60%, from about 50% to about 70%, from about 50% to about 80%, from about 50% to about 90%, from about 50% to about 100%, from about 60% to about 70%, from about 60% to about 80%, a from about 60% to about 90%, from about 60% to about 100%, %, from about 70% to about 80%, from about 70% to about 90%, from about 70% to about 100%, from about 80% to about 90%, from about 80% to about 100%, or from about 90% to about 100% of X in the covalent organic framework comprising repeating units of a moiety comprising Formula (I) is NR³.

In some embodiments, providing a covalent organic framework comprising repeating units of a moiety comprising Formula (II) comprises providing a dialdehyde comprising a moiety of Formula (V):

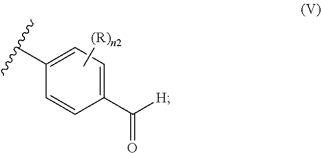

providing an amine comprising a moiety of Formula (VI):

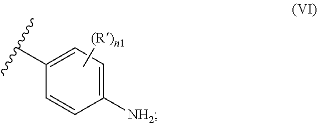

and
reacting the dialdehyde comprising a moiety of Formula (V) with the amine comprising a moiety of Formula (VI) under conditions effective to produce the covalent organic framework comprising repeating units of a moiety comprising Formula (II).

In some embodiments, the amine has a Formula (VIa) or Formula (VIb):

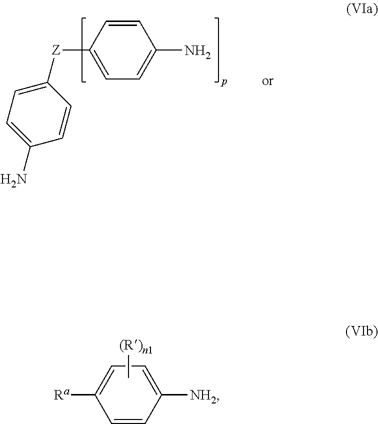

where
Z is selected from the group consisting of C, CH,

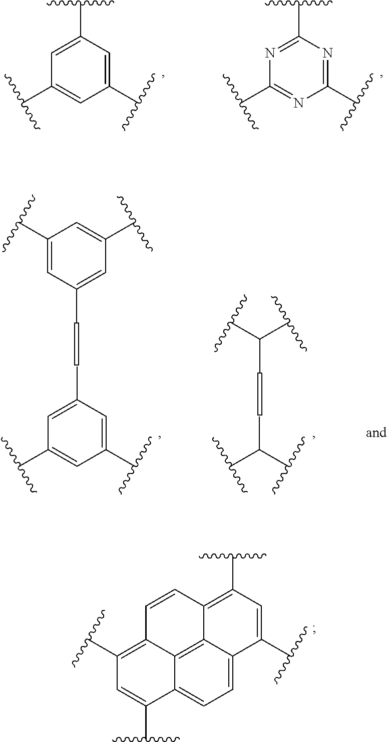

p is 2 or 3;
R^{a} is NH₂ or

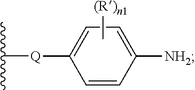

and
Q is optional, and if present, is —NHC(O)— or —C(O)NH—.

In some embodiments, the amine is selected from the group consisting of

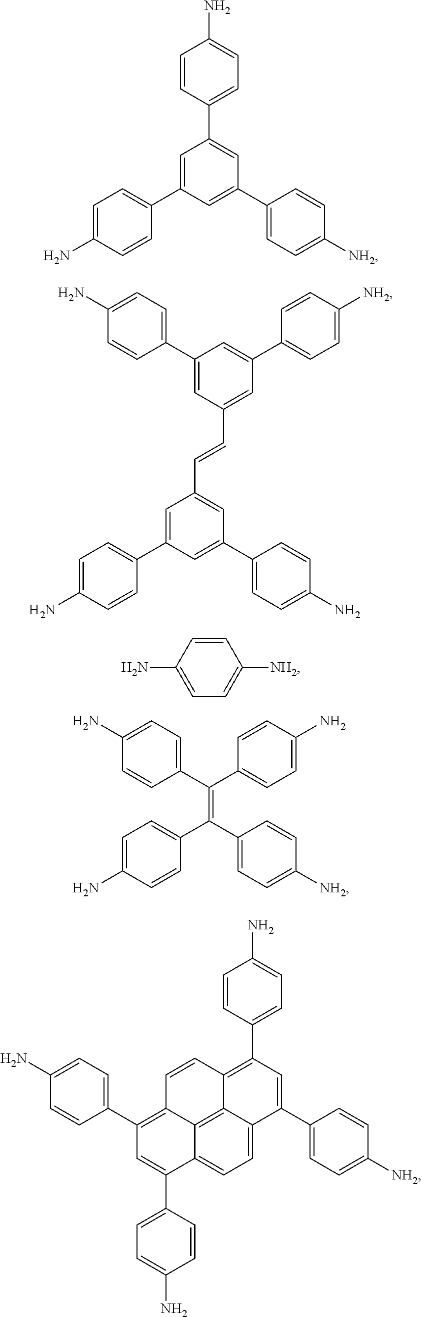

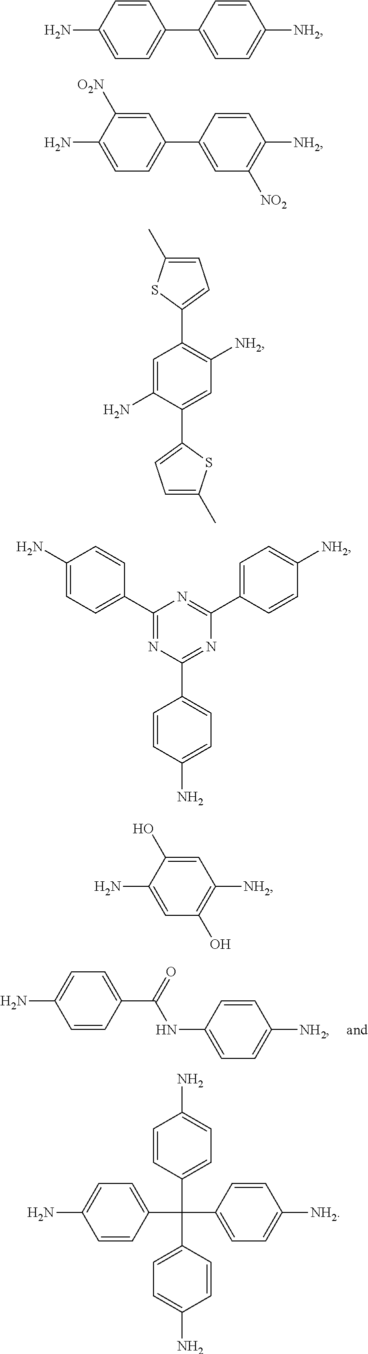

In some embodiments, the aldehyde has a Formula (Va)

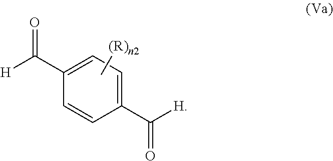

In some embodiments, the aldehyde is

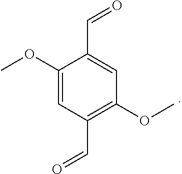

Another aspect of the present disclosure relates to a method of making a modified covalent organic framework comprising repeating units of a moiety comprising Formula (I′a):

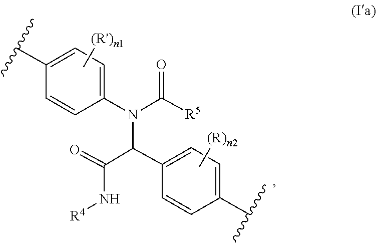

where
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R⁴ is —(CH₂)ₘC(O)NH(CH₂)ₖNH₂ substituted 1 to 3 times with R⁷;
R⁵ is C₁₋₁₂ alkyl substituted with R⁶;
R⁶ is —C(O)NH(CH₂)ₖNH₂ substituted 1 to 3 times with R⁷;
R⁷ is —(CH₂)ₖ₁NH₂ substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5, or 6; and
k₇ is 1, 2, 3, 4, 5, or 6.

This method comprises providing a covalent organic framework comprising repeating units of a moiety comprising Formula (I′b):

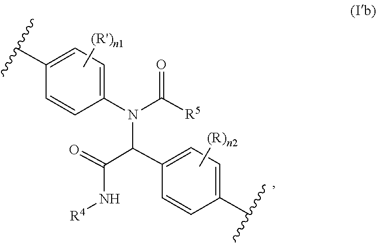

where
R⁴ is —(CH₂)ₘC(O)NH(CH₂)ₖNH₂ substituted 1 to 3 times with —(CH₂)ₖ₁NH₂;
R⁵ is C₁₋₁₂ alkyl substituted with R⁶; and
R⁶ is —C(O)NH(CH₂)ₖNH₂ substituted 1 to 3 times with —(CH₂)ₖ₁NH₂; and reacting the covalent organic framework comprising repeating units of a moiety comprising Formula (I′b) under conditions effective to produce the modified covalent organic framework.

In some embodiments, reacting comprises providing an acylating reagent, and reacting the covalent organic framework comprising repeating units of a moiety comprising Formula (I′b) with the acylating reagent to produce the modified covalent organic framework. Any suitable acylating agent can be used. Suitable acylating agents include, but are not limited to,

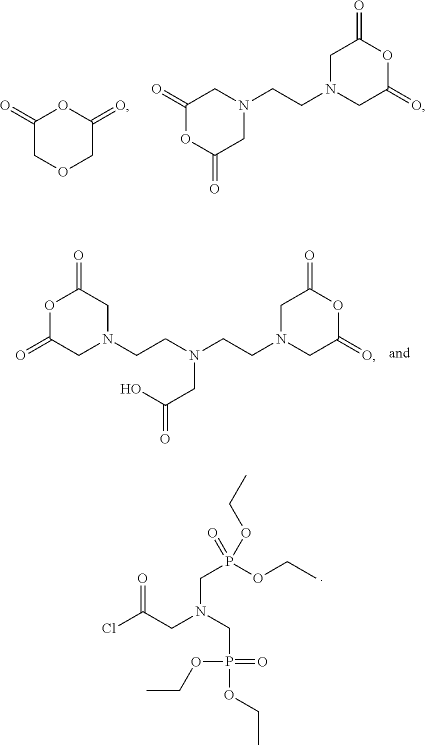

In one embodiment, the acylating reagent is a compound of Formula (VII):

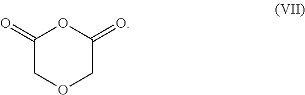

In some embodiments, the reacting of the covalent organic framework comprising repeating units of a moiety comprising Formula (I′b) with the acylating reagent is carried out at a temperature from about 20° C. to about 200° C. For example, in some embodiments, the reacting is carried out at a temperature from about 40° C. to about 180° C., about 60° C. to about 160° C., about 70° C. to about 140° C., about 80° C. to about 120° C., or about 90° C. to about 110° C.

In some embodiments, the reacting of the covalent organic framework comprising repeating units of a moiety comprising Formula (I′b) with the acylating reagent is carried out for at least 6 hours. For example, in some embodiments, the reacting is carried out for at least for at least 6 hours, at least 8 hours, at least 10 hours, at least 12 hours, at least 14 hours, at least 16 hours, or at least 24 hours.

In some embodiments, the providing a covalent organic framework comprising repeating units of a moiety comprising Formula (I′b) comprises providing a covalent organic framework comprising repeating units of a moiety comprising Formula (I″):

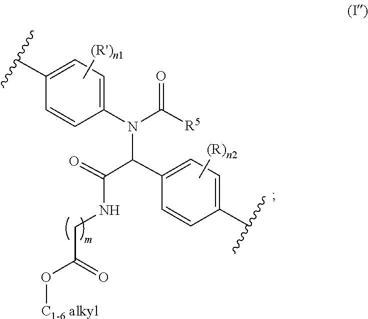

providing a compound of Formula (VIII):

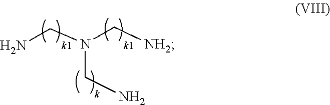

and reacting the covalent organic framework comprising repeating units of a moiety comprising Formula (I″) with the compound of Formula (VIII) to produce the covalent organic framework comprising repeating units of a moiety comprising Formula (I′b).

In some embodiments, the reacting of the covalent organic framework comprising repeating units of a moiety comprising Formula (I″) with the compound of Formula (VIII) is carried out at a temperature from about 20° C. to about 200° C. For example, in some embodiments, the reacting is carried out at a temperature from about about 40° C. to about 180° C., about 40° C. to about 160° C., about 40° C. to about 140° C., about 40° C. to about 120° C., about 40° C. to about 100° C., about 40° C. to about 80° C., about 50° C. to about 100° C., about 50° C. to about 90° C., about 50° C. to about 80° C., or about 50° C. to about 70° C.

In some embodiments, the reacting of the covalent organic framework comprising repeating units of a moiety comprising Formula (I″) with the compound of Formula (VIII) is carried out for at least 6 hours. For example, in some embodiments, the reacting is carried out for at least for at least 6 hours, at least 8 hours, at least 10 hours, at least 12 hours, at least 14 hours, at least 16 hours, or at least 24 hours.

In some embodiments, providing a covalent organic framework comprising repeating units of a moiety comprising Formula (I″) comprises providing a covalent organic framework comprising repeating units of a moiety comprising imine groups of Formula (II):

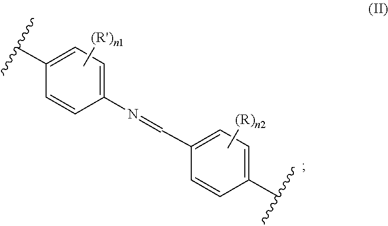

and
reacting the covalent organic framework comprising repeating units of a moiety comprising imine groups of Formula (II) under conditions effective to produce the covalent organic framework comprising repeating units of a moiety comprising Formula (I″).

In some embodiments, the reacting comprises providing a compound of Formula (IIIa):

C≡N—(CH₂)ₘC(O)OC₁₋₆ alkyl (IIIa);
providing a compound of Formula (IV):

R⁵COOH (IV); and
reacting the covalent organic framework comprising repeating units of a moiety comprising imine groups of Formula (II) with the compound of Formula (IIIa) and the compound of Formula (IV) to produce the covalent organic framework comprising repeating units of a moiety comprising Formula (I″).

The above disclosure is general. A more specific description is provided below in the following examples. The examples are described solely for the purpose of illustration and are not intended to limit the scope of the present disclosure. Changes in form and substitution of equivalents are contemplated as circumstances suggest or render expedient. Although specific terms have been employed herein, such terms are intended in a descriptive sense and not for purposes of limitation.

### EXAMPLES

The following Examples are presented to illustrate various aspects of the present disclosure, but are by no means intended to limit its scope.

### Example 1—Materials and Instrumentation for Examples 2-3

### Materials

Mesitylene and dioxane were purchased from Millipore Sigma. Methanol, acetone, tetrahydrofuran (THF), n-hexane, and acetic acid were purchased from Fisher. 4-Aminophenylboronic acid pinacol ester, 3,5-dibromobenzaldehyde, and 4-pentynoic acid were purchased from Combi-Blocks. 3-Bromopropionic acid, cyclohexyl isocyanide, palladium(0) tetrakis(triphenylphosphine), and 4-pentenoic acid were purchased from Oakwood Chemical. 1,3,5-Tris(4-aminophenyl)benzene (TAPB) (Mancheno et al., “Introduction to Covalent Organic Frameworks: An Advanced Organic Chemistry Experiment,” J. Chem. Educ., 96(8):1745-1751 (2019), which is hereby incorporated by reference in its entirety), (E)-3,3′,5,5′-tetrakis(4-aminophenyl)stilbene (TAPS) (Lyle et al., “Multistep Solid-State Organic Synthesis of Carbamate-Linked Covalent Organic Frameworks,” J. Am. Chem. Soc., 141(28):11253-11258 (2019), which is hereby incorporated by reference in its entirety), 2,5-dimethoxyterephthalaldehyde (DMTP) (Kang et al., “Aggregated Structures of Two-Dimensional Covalent Organic Frameworks,” J. Am. Chem. Soc., 144(7):3192-3199 (2022), which is hereby incorporated by reference in its entirety), 3-azidopropionic acid (Schmitz et al., “Cathepsin B Inhibitors: Combining Dipeptide Nitriles with an Occluding Loop Recognition Element by Click Chemistry,” ACS Med. Chem. Lett., 7(3):211-216 (2016), which is hereby incorporated by reference in its entirety), and 1-(2-methoxyphenyl)-N-phenylmethanimine (Cardinale et al., “Photoredox-Catalyzed Synthesis of α-Amino Acid Amides by Imine Carbamoylation,” Org. Lett., 24(2):506-510 (2022), which is hereby incorporated by reference in its entirety) were synthesized according to previously reported protocols.

### Sonication

Sonication was performed with a Branson 2510 Ultrasonic Cleaner with a frequency of 40 kHz.

### Fourier-Transform Infrared (FTIR) Spectroscopy

Infrared spectra were collected using an Agilent Technologies Cary 600 Series FTIR Spectrometer equipped with a Diamond ATR accessory.

### Thermogravimetric Analysis (TGA)

TGA was measured on Netzsch STA449 F1 over the temperature range from 40 to 900° C. The analysis was carried out under an argon atmosphere with a heating rate of 10° C./min using an empty Al₂O₃ crucible as the reference.

### Powder X-Ray Diffraction (PXRD)

Powder X-ray diffraction patterns were collected using a Bruker D8-Advance diffractometer in parallel beam geometry employing Cu Kα (1.5418 Å) line-focused radiation at 40 kV/40 mA power and equipped with a position-sensitive detector. Covalent organic framework (COF) samples were loaded on zero background sample holders (MTI Corporation, ZeroSi32D20C1cavity10D) by dropping the powders from a metal spatula and leveling the sample. In the case of TAPS-DMTP-COF and its derivatives, powders were drop-casted onto the sample holder using THF to disrupt the preferred orientation of crystals. Samples were rotated at a rate of 15 rpm. Data were collected from 1.0° 2θ to 30.0° 2θ with 0.03° 2θ per step (983 steps) and an exposure time of 7 s per step, for a total acquisition time of 126 min.

### X-Ray Photoelectron Spectra (XPS)

The XPS measurements were performed using a Kratos Amicus/ESCA 3400 instrument. The sample was irradiated with 240 W unmonochromated Mg Kα x-rays, and photoelectrons emitted at 0° from the surface normal were energy analyzed using a DuPont type analyzer. The pass energy was set at 150 eV and a Shirley baseline was removed from the spectra. CasaXPS was used to process raw data files.

### Nuclear Magnetic Resonance (NMR) Spectroscopy

¹H NMR and ¹³C NMR spectra were recorded at 25° C. on Varian 400 MHz. The spectra were calibrated using residual solvent as internal reference (CDCl₃: 7.26 ppm for ¹H NMR, 77.00 ppm for ¹³C NMR).

### Scanning Electron Microscopy (SEM)

SEM images were taken on JEOL JSM-IT200 Scanning Electron Microscope. Samples were dispersed in ethanol or acetone and drop-casted on the silicon wafer substrate.

### Gas Sorption Isotherms

Nitrogen physisorption experiments were conducted on a Micromeritics Tristar Analyzer using 80-100 mg samples in dried and tared analysis tubes equipped with filler rods and capped with a Transeal. Samples were heated to 100° C. for 6 hours and degassed using nitrogen gas flow. Each tube was weighed again to determine the mass of the activated sample and transferred to the analysis port of the instrument. UHP-grade (99.999% purity) N₂ was used for all sorption measurements. N₂ sorption isotherms were generated by incremental exposure to nitrogen up to 760 mmHg (1 atm) at 77 K (liquid N₂ bath). The Brunauer-Emmett-Teller (BET) method was utilized to calculate the specific surface areas.

### Conventional Solid-State Spinning NMR Spectroscopy

Room-temperature magic angle spinning (MAS) ¹³C solid-state NMR experiments were performed with a 9.4 T widebore Bruker NMR spectrometer (n₀ (¹³C) 100.7 MHz) equipped with an Avance III HD console, and a Bruker 4 mm MAS probe configured in double resonance ¹H-¹³C mode. ¹³C solid-state NMR spectra were acquired with cross-polarization (CP) for signal enhancement (Pines et al., “Proton-Enhanced Nuclear Induction Spectroscopy. A Method for High Resolution NMR of Dilute Spins in Solids,” Chem. Phys., 56(4):1776-1777 (1972); Schaefer and Stejskal, “Carbon-13 Nuclear Magnetic Resonance of Polymers Spinning at the Magic Angle,” J. Am. Chem. Soc., 98(4):1031-1032 (1976), which are hereby incorporated by reference in their entirety). ¹H-¹³C CP matching conditions were optimized on an external standard of adamantane. CP experiments on COF materials used a 2 ms CP contact time. 65,536 scans were acquired each for the kag-COF and kag-Ugi-COF-OAc-[1.0]. A 1.0 s recycle delay was employed in both cases to maximize sensitivity. ¹H saturation recovery experiments indicated that the ¹H T₁ was approximately 0.7 s for both COF materials. The magic angle spinning (MAS) frequency was 10 kHz. All ¹H CP spin-lock RF fields were linearly ramped from 90% to 100% amplitude (Metz et al., “Ramped-Amplitude Cross Polarization in Magic-Angle-Spinning NMR,” J. magn. reson., Ser. A, 110(2):219-227 (1994), which is hereby incorporated by reference in its entirety) to broaden the Hartman-Hahn match condition. The ¹H-¹³C CP experiments used spin lock pulses with RF fields of ca. 34 kHz and 44 kHz for ¹H and ¹³C, respectively. SPINAL-64 heteronuclear decoupling (Fung et al., “An Improved Broadband Decoupling Sequence for Liquid Crystals and Solids,” J. Magn. Reson., 142(1):97-101 (2000), which is hereby incorporated by reference in its entirety) with a ¹H RF field of 100 kHz was applied during the acquisition.

### Dynamic Nuclear Polarization Solid-State NMR Spectroscopy

In general, ca. 10 mg of hex-COF samples were weighed out and placed in a watch glass, where ca. 20 mL of a 16 mM TEKPol TCE solution was added by impregnation. The impregnated sample was then packed into a 3.2 mm DNP sapphire rotor, then capped with a Teflon insert and zirconia drive cap. ¹³C and ¹⁵N SSNMR DNP experiments were conducted on a 9.4 T Bruker 400 MHz/263 GHz DNP NMR system (Rosay et al., “Solid-State Dynamic Nuclear Polarization at 263 GHz: Spectrometer Design and Experimental Results,” Phys. Chem. Chem. Phys., 12(22):5850-5860 (2010), which is hereby incorporated by reference in its entirety) equipped with a Bruker Avance III console and a Bruker 3.2 mm HXY MAS DNP-NMR probe configured in triple resonance HCN mode. The main magnetic field was set on a standard sample of TEKPol TCE solution so that microwave irradiation gave a maximum positive DNP enhancement. The sample temperature was approximately 110 K for all experiments. The magic angle spinning (MAS) frequency was 10 kHz. ¹H-¹⁵N cross polarization (CP) was optimized on an external standard of ¹³C, ¹⁵N labeled glycine. All ¹H CP spin-lock RF fields were linearly ramped from 90% to 100% amplitude (Metz et al., “Ramped-Amplitude Cross Polarization in Magic-Angle-Spinning NMR,” J. magn. reson., Ser. A, 110(2):219-227 (1994), which is hereby incorporated by reference in its entirety) to broaden the Hartman-Hahn match condition. The ¹H-¹³C CP experiments used spin lock pulses with RF fields of ca. 65 kHz and 50 kHz for ¹H and ¹³C, respectively, and a 2 ms contact time. The ¹H-¹⁵N CP experiments used spin lock pulses with RF fields of ca. 70 kHz and 42 kHz for ¹H and ¹⁵N, respectively, and 5 ms contact times. Recycle delay of 7.5 s and 6.5 s were used for hex-COF and hex-Ugi-COF-OAc-[1.0], respectively. These recycle delays were chosen for optimal sensitivity, based upon DNP signal build-up times measured with ¹H-¹³C CP saturation recovery experiments (FIG. 5). 5,120 and 10,240 scans were acquired for hex-COF and hex-Ugi-COF-OAc-[1.0], respectively. 100 kHz ¹H RF field SPINAL-64 heteronuclear decoupling (Fung et al., “An Improved Broadband Decoupling Sequence for Liquid Crystals and Solids,” J. Magn. Reson., 142(1):97-101 (2000), which is hereby incorporated by reference in its entirety) was applied during all evolution periods and during the acquisition of ¹³C and ¹⁵N. A ¹³C CPMAS saturation recovery sequence was used for the measurements of ¹H DNP build-up times (T₁/T_{DNP}).

### Example 2—Synthetic Procedures

### Synthesis of TAPB-DMTP-COF (Hex-COF)

Hex-COF was prepared according to a previously reported procedure (Xu et al., “Stable, Crystalline, Porous, Covalent Organic Frameworks as a Platform for Chiral Organocatalysts,” Nat. Chem., 7(11):905-912 (2015), which is hereby incorporated by reference in its entirety) with some modifications. TAPB (56.2 mg, 0.16 mmol) and DMTP (46.6 mg, 0.24 mmol) were charged in a prescored glass ampule (20 mL). Mesitylene (3.2 mL) and dioxane (0.8 mL) were added, and the solution was sonicated for 5 min to produce a homogenous suspension. Acetic acid (0.4 mL, 6.0 M) was added. The ampule was degassed through three freeze-pump-thaw cycles, flash frozen at 77 K (liquid N₂ bath), evacuated to an internal pressure of 100 mTorr, and flame sealed. The reaction mixture was allowed to warm up to room temperature and heated at 120° C. for 72 hours without disturbance. Upon cooling the reaction to room temperature, the yellow precipitate was isolated by filtration and washed with acetone. While still activated, the product was transferred to the Soxhlet extractor for further purification with THF for 24 hours. Afterward, the powder was collected by filtration, washed with acetone, and dried at 100° C. under reduced pressure (100 mTorr) for 24 hours, affording the product as a bright yellow powder (80 mg, 81% yield).

### Synthesis of TAPS-DMTP-COF (Kag-COF)

Kag-COF was prepared according to a previously reported procedure (Lyle et al., “Multistep Solid-State Organic Synthesis of Carbamate-Linked Covalent Organic Frameworks,” J. Am. Chem. Soc., 141(28):11253-11258 (2019), which is hereby incorporated by reference in its entirety) with some modifications. TAPS (87.2 mg, 0.16 mmol) and DMTP (62.1 mg, 0.32 mmol) were charged in a prescored glass ampule (20 mL). Mesitylene (2.8 mL) and dioxane (1.2 mL) were added, and the solution was sonicated for 5 min to produce a homogenous suspension. Acetic acid (0.4 mL, 6.0 M) was added. The ampule was degassed through three freeze-pump-thaw cycles, flash frozen at 77 K (liquid N₂ bath), evacuated to an internal pressure of 100 mTorr, and flame sealed. The reaction mixture was allowed to warm up to room temperature and heated at 120° C. for 72 hours without disturbance. Upon cooling the reaction to room temperature, the yellow precipitate was isolated by filtration and washed with acetone. While still activated, the product was transferred to the Soxhlet extractor for further purification with THF for 24 hours. Afterward, the powder was collected by filtration, washed with acetone, and dried at 100° C. under reduced pressure (100 mTorr) for 24 hours, affording the product as a bright yellow powder (120 mg, 83% yield).

### Ugi Multicomponent Reaction

Synthesis of N-Cyclohexyl-2-phenyl-2-(N-(2-methoxy-phenyl)acetamido)acetamide (Model Compound)

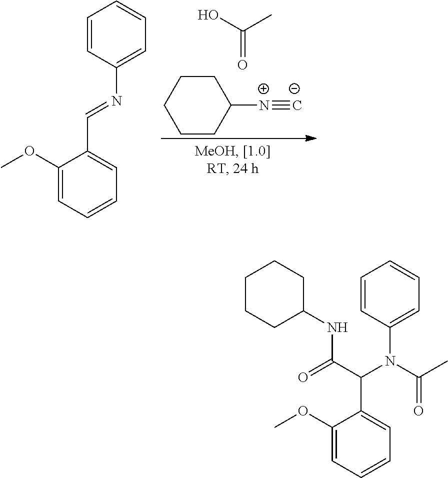

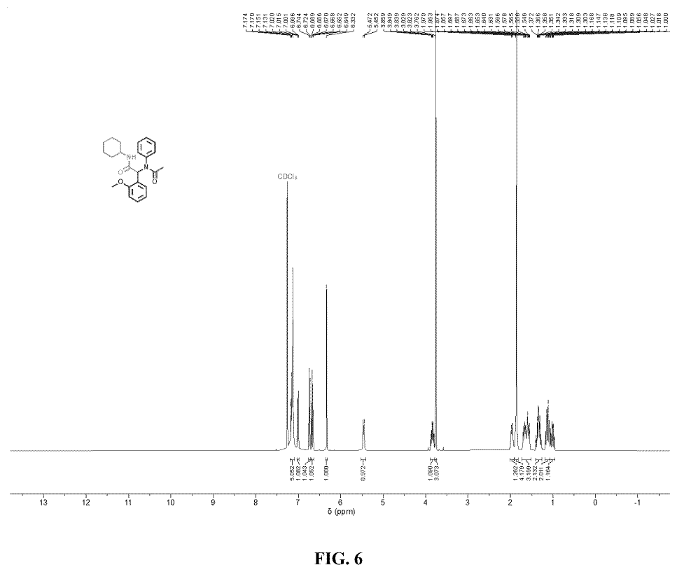

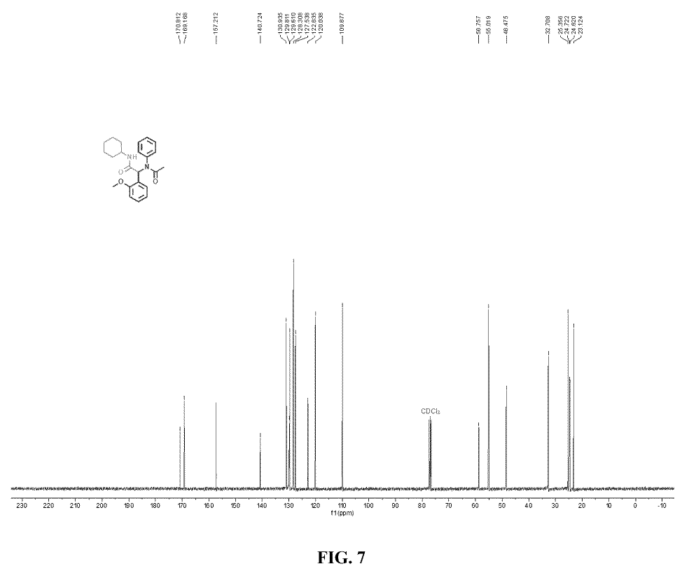

1-(2-Methoxyphenyl)-N-phenylmethanimine (105 mg, 0.5 mmol) was charged to a 1-dram glass vial. Methanol (0.41 mL), acetic acid (30 mg, 0.5 mmol, 1.0 equiv), and cyclohexyl isocyanide (54 mg, 0.5 mmol, 1.0 equiv) were added in this order, and the vial was sealed with a Teflon cap. The precipitate formed within minutes, but the reaction was allowed to proceed for additional 24 hours at room temperature. The product was isolated by filtration, washed with n-hexane, and dried under reduced pressure, affording the model compound as a white solid (155 mg, 96%). ¹H NMR (400 MHz, CDCl₃): δ 7.10-7.18 (m, 5H), 7.01 (dd, J=7.6, 2.0 Hz, 1H), 6.73 (d, J=8.0 Hz, 1H), 6.67 (td, J=7.6, 1.2 Hz, 1H), 6.33 (s, 1H), 5.46 (d, J=8.0 Hz, 1H), 3.89-3.78 (m, 1H), 3.76 (s, 3H), 2.01-1.92 (m, 1H), 1.87 (m, 1H), 1.86 (s, 3H), 1.73-1.52 (m, 3H), 1.41-1.26 (m, 2H), 1.18-1.07 (m, 2H), 1.07-0.94 (m, 1H) (FIG. 6); ¹³C NMR (101 MHz, CDCl₃): δ 170.8, 169.2, 157.2, 140.7, 130.9, 129.8, 129.6, 128.3, 127.5, 122.8, 120.0, 109.9, 58.8, 55.0, 48.5, 32.7, 25.4, 24.7, 24.6, 23.1 (FIG. 7); MS (HR-ESI), m/z calcd. C₂₃H₂₉N₂O₃ [M-H]⁺ 381.2178, found: 381.2177.

Synthesis of TAPB-DMTP-Ugi-COF-OAc-[1.0](hex-Ugi-COF-OAc-[1.0])

Hex-COF (10.0 mg, 0.0085 mmol, MW=1177.38 g/mol) was charged to a 1-dram glass vial. Methanol (0.21 mL), acetic acid (15 mg, 0.25 mmol, 5.0 equiv×6 imine bonds per unit cell), and cyclohexyl isocyanide (28 mg, 0.25 mmol, 5.0 equiv×6 imine bonds per unit cell) were added in this order, and the vial was sealed with a Teflon cap. Hence, the concentration of the acid and the isocyanide reaction components was kept at 1.0 M each. The reaction was placed in a metal block preheated to 40° C. and maintained at that temperature for 24 hours without stirring. The progress of the reaction was monitored using the color-responsive nature of the conjugated COF framework: upon initial addition of the acetic acid, the material turned dark red (imine protonation), followed by a gradual color change to light orange or yellow (Ugi adduct formation), which indicated the reaction competition. The product was isolated by filtration, washed with a copious amount of acetone, and dried at 100° C. under reduced pressure (100 mTorr) for 24 hours, affording hex-Ugi-COF-OAc-[1.0] as a yellow powder (12 mg).

Synthesis of TAPB-DMTP-Ugi-COF-OAc-[1.5](hex-Ugi-COF-OAc-[1.5])

The product was prepared according to the previously described procedure using hex-COF (10 mg, 0.0085 mmol), methanol (0.19 mL), acetic acid (23 mg, 0.38 mmol, 7.5 equiv×6 imine bonds per unit cell), and cyclohexyl isocyanide (42 mg, 0.38 mmol, 7.5 equiv×6 imine bonds per unit cell). Hence, the concentration of the acid and the isocyanide reaction components was kept at 1.5 M each. The reaction afforded hex-Ugi-COF—OAc-[1.5] as a yellow powder (14 mg).

### Synthesis of TAPB-DMTP-Ugi-COF Derivatives (Hex-Ugi-COF-R)

Functionalized hex-Ugi-COF derivatives were prepared according to the previously described procedure using hex-COF (10 mg, 0.0085 mmol), methanol (0.19 mL), corresponding carboxylic acid (3-azidopropionic acid, 4-pentynoic acid, or 4-pentenoic acid) (0.38 mmol, 7.5 equiv×6 imine bonds per unit cell), and cyclohexyl isocyanide (42 mg, 0.38 mmol, 7.5 equiv×6 imine bonds per unit cell). In each case, the concentration of the acid and the isocyanide reaction components was kept at 1.5 M each. The reaction afforded hex-Ugi-COF—N₃ (14 mg), hex-Ugi-COF—C≡C (14 mg), or hex-Ugi-COF—C═C (14 mg) as yellow powders.

Synthesis of TAPS-DMTP-Ugi-COF-OAc-[1.0](kag-Ugi-COF-OAc-[1.0]).

Kag-COF (7.6 mg, 0.0085 mmol, MW=897.05 g/mol) was charged to a 1-dram glass vial. Methanol (0.21 mL), acetic acid (15 mg, 0.25 mmol, 15.0 equiv×2 imine bonds per unit cell), and cyclohexyl isocyanide (28 mg, 0.25 mmol, 15.0 equiv×2 imine bonds per unit cell) were added in this order, and the vial was sealed with a Teflon cap. Hence, the concentration of the acid and the isocyanide reaction components was kept at 1.0 M each. The reaction was placed in a metal block preheated to 40° C. and maintained at that temperature for 24 hours without stirring. The progress of the reaction was monitored using the color-responsive nature of the conjugated COF framework: upon initial addition of the acetic acid, the material turned dark red (imine protonation), followed by a gradual color change to light orange or yellow (Ugi adduct formation), which indicated the reaction completion. The product was isolated by filtration, washed with a copious amount of acetone, and dried at 100° C. under reduced pressure (100 mTorr) for 24 hours, affording hex-Ugi-COF-OAc-[1.0] as a yellow powder (12 mg).

Synthesis of TAPS-DMTP-Ugi-COF Derivatives (kag-Ugi-COF-R)

Functionalized kag-Ugi-COF derivatives were prepared according to the previously described procedure using kag-COF (7.6 mg, 0.0085 mmol), methanol (0.21 mL), corresponding carboxylic acid (3-azidopropionic acid, 4-pentynoic acid, or 4-pentenoic acid) (0.25 mmol, 15.0 equiv×2 imine bonds per unit cell), and cyclohexyl isocyanide (28 mg, 0.25 mmol, 15.0 equiv×2 imine bonds per unit cell). In each case, the concentrations of the acid and the isocyanide reaction components were kept at 1.0 M each. The reaction afforded kag-Ugi-COF—N₃ (8 mg), kag-Ugi-COF—C≡C (8 mg), or kag-Ugi-COF—C═C (8 mg) as yellow powders.

### Example 3—Results and Discussion for Examples 1-2

A typical Ugi reaction involves the multicomponent reaction of imine, isocyanide, and carboxylic acid reagents to generate a bis-amide product (Rocha et al., “Review on the Ugi Multicomponent Reaction Mechanism and the Use of Fluorescent Derivatives as Functional Chromophores,” ACS Omega, 5(2):972-979 (2020); Fouad et al., “Two Decades of Recent Advances of Ugi Reactions: Synthetic and Pharmaceutical Applications,” RSC Adv., 10(70):42644-42681 (2020), which are hereby incorporated by reference in their entirety). High concentrations of the above components in polar protic solvents usually lead to higher yields of the Ugi adduct (Domling et al., “Multicomponent Reactions with Isocyanides,” Angew. Chem., Int. Ed., 39(18):3168-3210 (2000); Marcaccini and Torroba, “The Use of the Ugi Four-Component Condensation,” Nat. Protoc., 2(3):632-639 (2007); Bradley et al., “Optimization of the Ugi Reaction Using Parallel Synthesis and Automated Liquid Handling,” J. Vis. Exp., 21:942 (2008), which are hereby incorporated by reference in their entirety). To test the feasibility of this reaction, N-cyclohexyl-2-phenyl-2-(N-(2-methoxy-phenyl)acetamido)acetamide (model compound) was first synthesized from N-(2-methoxybenzylidene)aniline, cyclohexyl isocyanide, and acetic acid in methanol at room temperature in 10 minutes with quantitative yield. Inspired by the preliminary success, these reaction conditions were applied to the COF materials, obtaining the TAPB-DMTP-Ugi-COF (hex-Ugi-COF) and TAPS-DMTP-Ugi-COF (kag-Ugi-COF). During optimization, it was found that the concentrations of isocyanide and carboxylic acid must be at least 1.0 M to achieve more than 50% functionalization of the porous materials. However, running the reaction at 2.0 M resulted in an almost complete conversion of the imine bonds to the Ugi bis-amide but lead to a PXRD-silent profile of the Ugi-COFs. Hence, the concentration of 1.5 M was identified to lead to the highest degree of functionalization while retaining the crystallinity of the material.

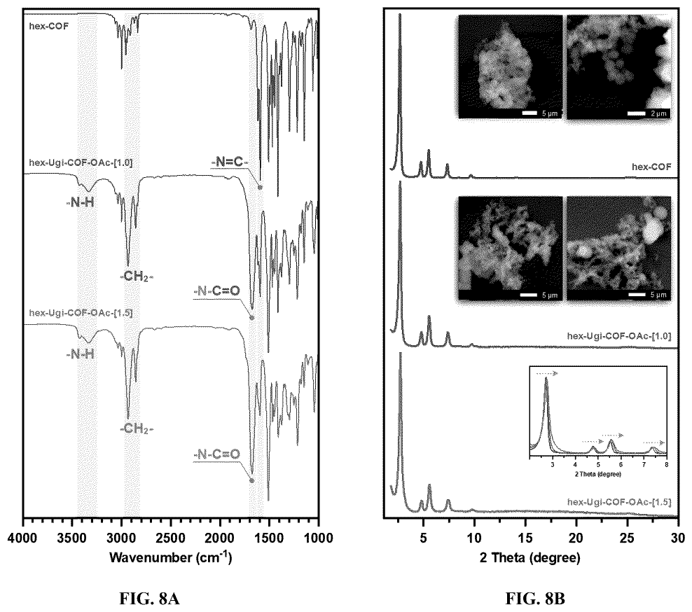

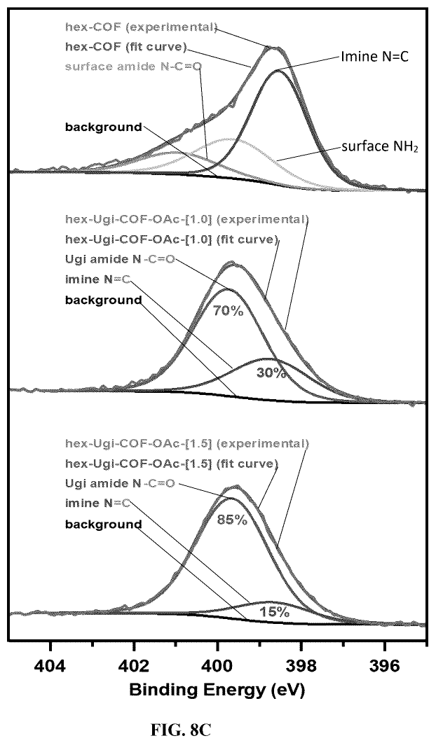

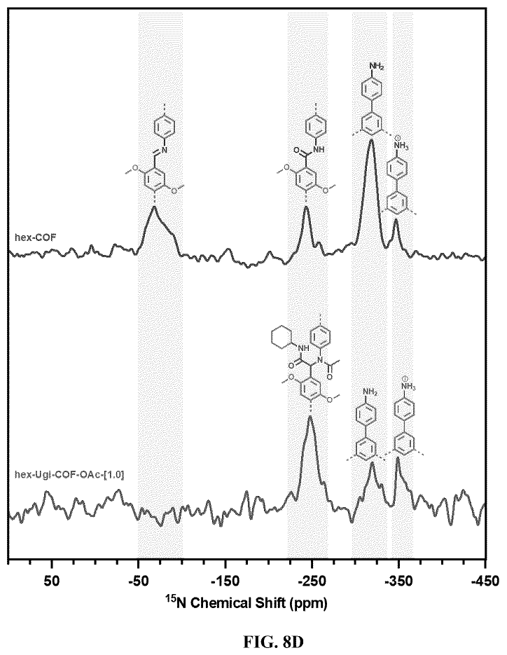

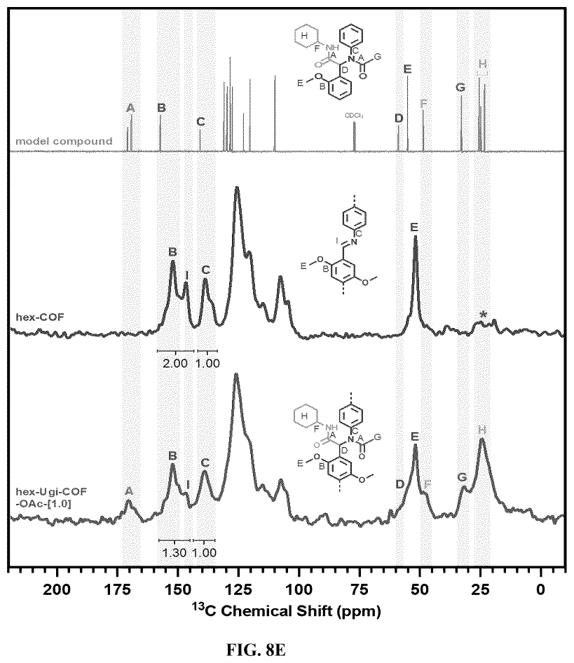

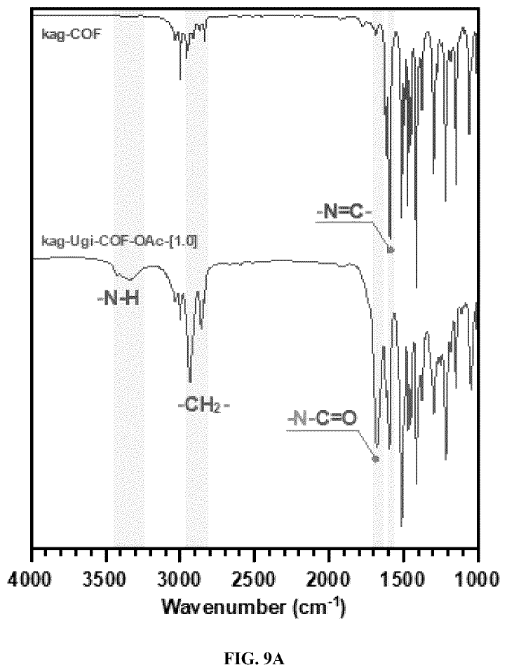

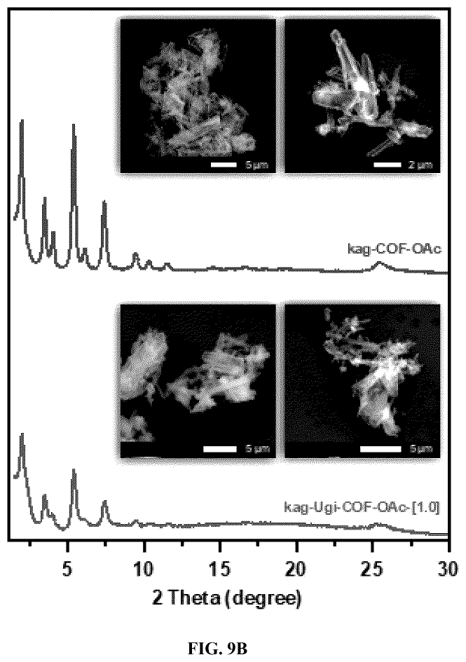

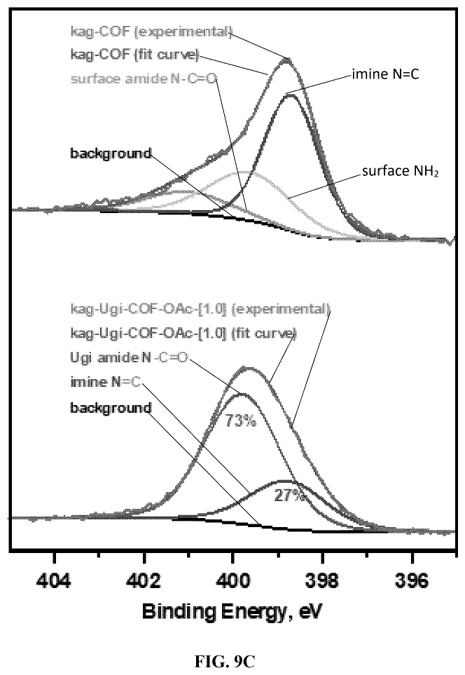

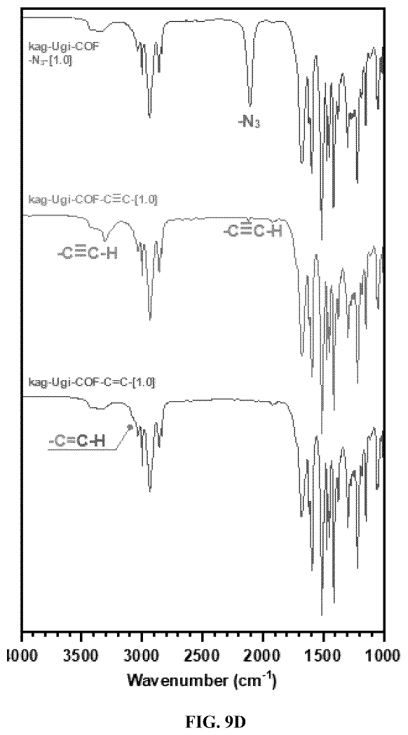

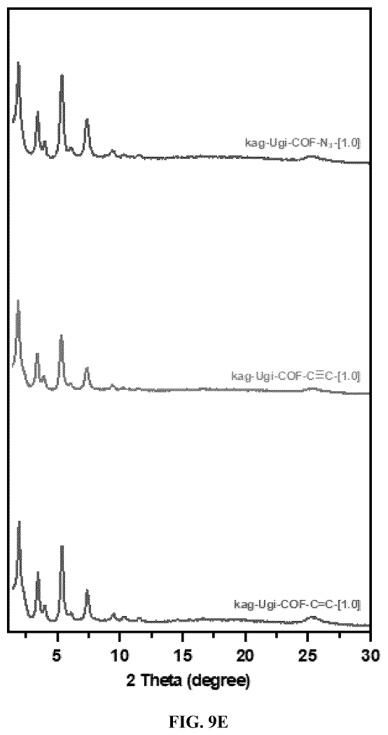

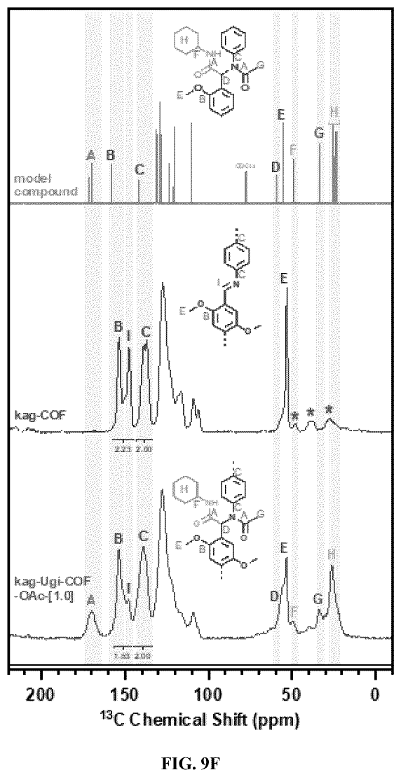

For routine assessment, Fourier-transform infrared (FTIR) spectroscopy was utilized to confirm the formation of the Ugi adduct at the backbone of the COFs. In the case of hex-Ugi-COF-OAc-[1.0] obtained from the reaction of hex-COF (FIG. 8A) in a 1.0 M solution of cyclohexyl isocyanide and acetic acid in methanol, the appearance of strong amide frequencies at 1672 cm⁴ (C═O) and 3331 cm⁻¹ (N—H) were observed (FIG. 8A), as well as strong frequencies at 2933 cm⁻¹ and 2855 cm⁻¹ corresponding to the aliphatic cyclohexyl group (CH₂). The attenuation of the imine stretch (C═N) at 1592 cm⁻¹ further supported the formation of the desired Ugi product. Increasing the concentration of the reaction components to 1.5 M resulted in a higher degree of functionalization, as supported by the intensified signals of the amide and cyclohexyl groups, as well as a further weakening of the imine stretch for the hex-Ugi-COF-OAc-[1.5](FIG. 8A). Similar FTIR spectra were obtained when the kag-COF (FIG. 9A) was subjected to a 1.0 M solution of acetic acid and cyclohexyl isocyanide in methanol to obtain kag-Ugi-COF (FIG. 9A). The presence of characteristic Ugi adduct features was confirmed, including the amide frequencies at 1677 cm⁻¹ (C═O) and 3336 cm⁻¹ (N—H), strong frequencies of the cyclohexyl group (CH₂) at 2932 cm⁻¹ and 2855 cm⁻¹, as well as weakening of the imine (C═N) frequency at 1590 cm⁻¹.

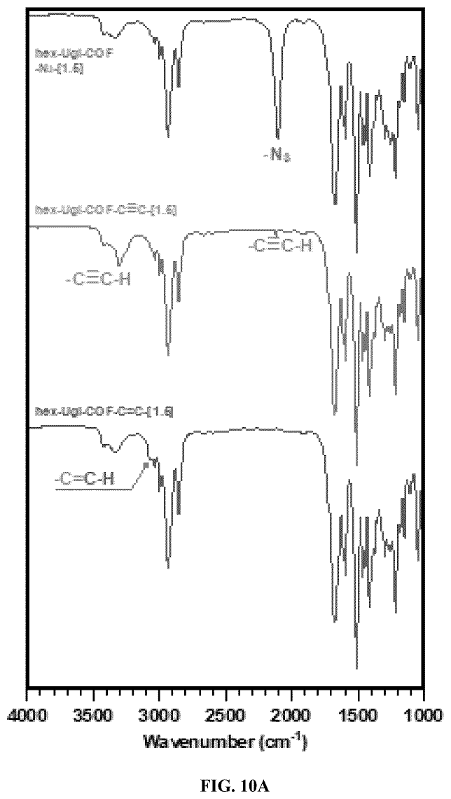

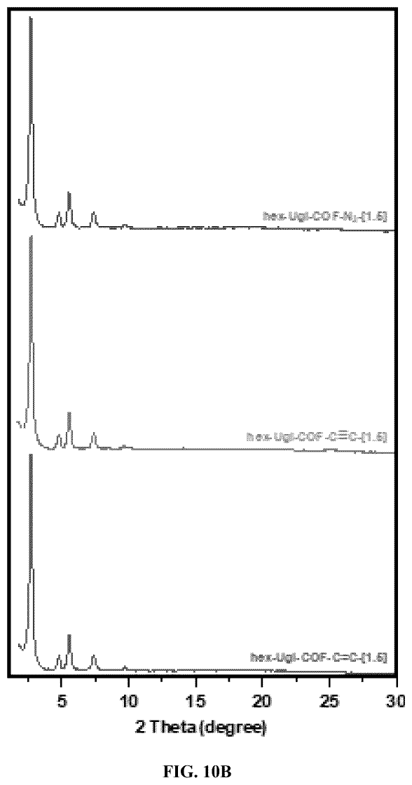

To introduce functional group handles in the COFs, the acetic acid was replaced with either 3-azidopropionic acid, 4-pentynoic acid, or 4-pentenoic acid in Ugi reactions with cyclohexyl isocyanide and hex-COF in a 1.5 M solution of methanol. In addition to the previously described FTIR features of the hex-Ugi-COF-OAc materials, the corresponding functionalized hex-Ugi-COF—N₃-[1.5] material showed a presence of strong azide frequency at 2103 cm⁻¹ (—N₃) (FIG. 10A). In the case of the hex-Ugi-COF—C═C-[1.5], a medium intensity frequency at 3303 cm⁻¹ (alkyne C—H) and a weak frequency at 2119 cm⁻¹ (C≡C) of the alkyne group (FIG. 10A) were observed. For the hex-Ugi-COF—C═C-[1.5], a weak frequency at 3064 cm⁻¹ (vinyl C—H) was observed, corresponding to the vinyl group (FIG. 10A). When azide, alkyne, and vinyl carboxylic acid derivatives were utilized in the Ugi reaction with cyclohexyl isocyanide and kag-COF in a 1.0 M solution of methanol, similar new FTIR frequencies were observed at 2103 cm⁻¹ (—N₃), 3306 cm⁻¹ (alkyne C—H) and 2120 cm⁻¹ (C—C), and 3070 cm⁻¹ (vinyl C—H), respectively for the kag-Ugi-COF—N₃-[1.0](FIG. 9D), kag-Ugi-COF—C≡C-[1.0](FIG. 9D), and kag-Ugi-COF—C═C-[1.0](FIG. 9D).

To confirm the retention of crystallinity throughout the Ugi reaction, powder X-ray diffraction (PXRD) patterns of as-synthesized hex-COF (FIG. 11A) and kag-COF (FIG. 11B) were collected, as well as their corresponding Ugi-modified derivatives. All major peaks and their strong intensities were preserved throughout the transformation for hex-COF (FIG. 8B) and its derivatives (FIG. 10B), as well as for the kag-COF (FIG. 9B) and its derivatives (FIG. 9E). A closer comparison of the juxtaposed PXRD patterns for the hex-COF and hex-Ugi-COF-OAc samples from 2° to 8° revealed subtle changes to the crystal structure. The following peaks, corresponding to the 100, 110, 200, and 210 facets, respectively, underwent higher angle shifts as the loading of the Ugi-adduct increased at the COF backbone (FIG. 8B, bottom inset): 2.69°→2.72°→2.75°, 4.770→4.80°→4.86°, 5.51°→5.57°→5.60°, 7.36°→7.39°→7.42°. This effect was attributed to the layer buckling (Emmerling et al., “Interlayer Interactions as Design Tool for Large-Pore COFs,” J. Am. Chem. Soc., 143(38):15711-15722 (2021); Kang et al., “Interlayer Shifting in Two-Dimensional Covalent Organic Frameworks,” J. Am. Chem. Soc., 142(30):12995-13002 (2020); Kang et al., “Aggregated Structures of Two-Dimensional Covalent Organic Frameworks,” J. Am. Chem. Soc., 144(7):3192-3199 (2022), which are hereby incorporated by reference in their entirety). As the density of the newly installed functional groups increases inside the bulk of the COF, the interlayer lattice stacking is disrupted. This disruption in the interlayer lattice stacking causes some layers to distort and/or shift, which is reflected by the transpositions of the in-plane diffraction patterns.

Scanning Electron Microscopy (SEM) imaging was utilized to assess the retention of morphology for COF material before and after the Ugi transformation. For as-synthesized hex-COF crystals, the material was isolated mostly as islands of spherical particles (FIG. 8B, top inset images). This morphology was maintained after the Ugi transformation for the hex-Ugi-COF-OAc-[1.0](FIG. 8B, middle inset images). As-synthesized kag-COF crystals were obtained in the rod-like shape (FIG. 9B, top inset images), and this morphology was preserved throughout the Ugi modification for the kag-Ugi-COF-OAc-[1.0](FIG. 9B, bottom inset images).

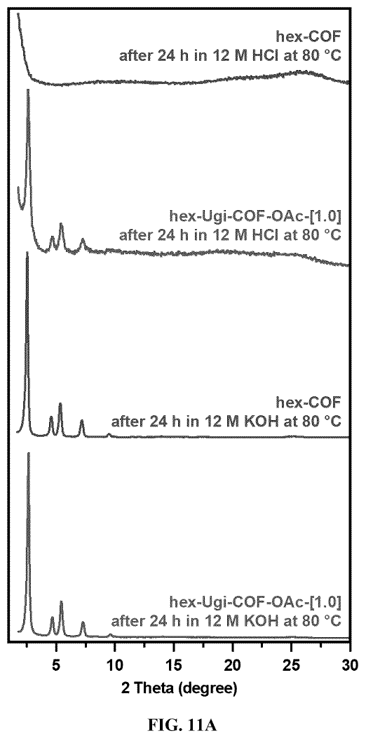

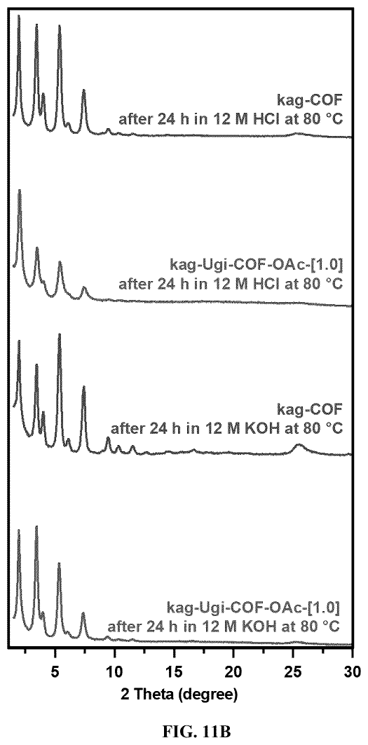

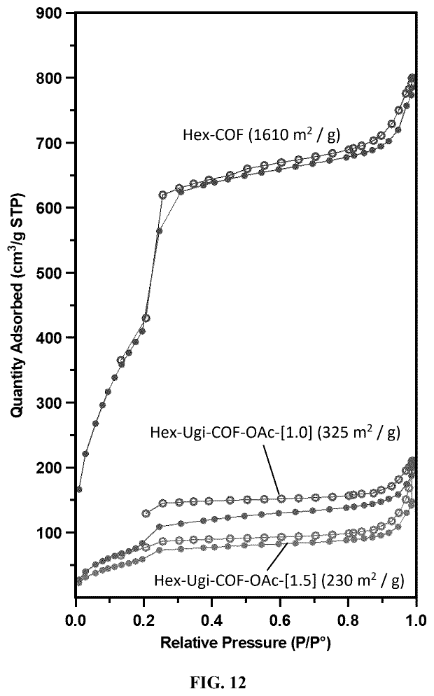

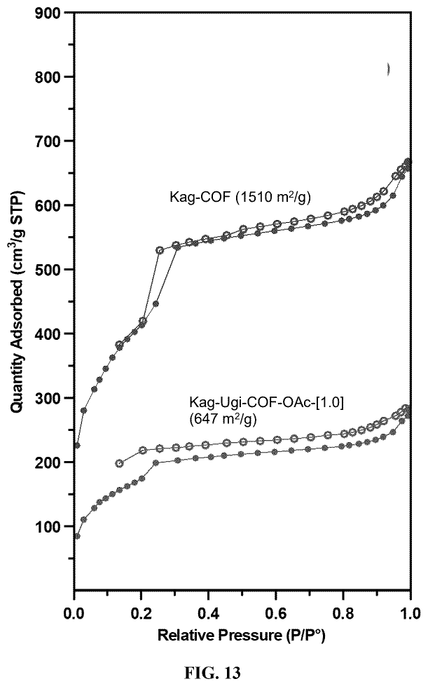

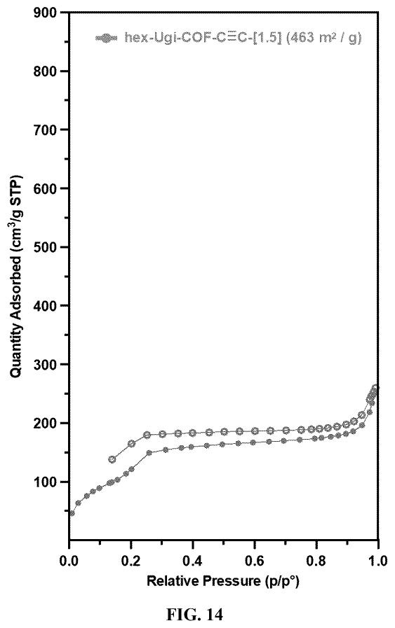

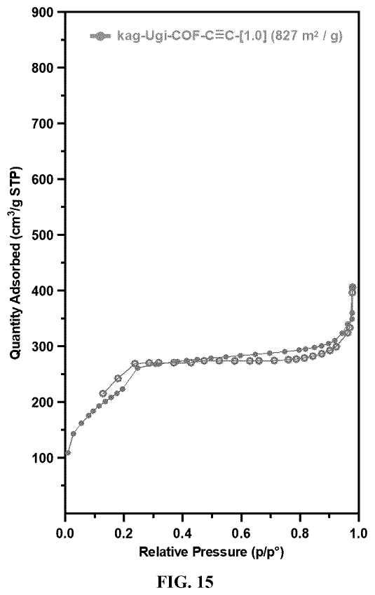

To better understand the effect of the Ugi transformation on the permanent porosity of the COFs, the Brunauer-Emmett-Teller (BET) measurements for the materials before and after the Ugi reaction were obtained. The BET surface area of as-synthesized hex-COF was measured at 1610 m²/g, while for the hex-Ugi-COF-OAc-[1.0] and hex-Ugi-COF—OAc-[1.5], the BET surface areas were measured at 325 and 230 m²/g, respectively (FIG. 12). As-synthesized kag-COF provided a BET surface area of 1510 m²/g, while for the kag-Ugi-COF-OAc-[1.0], the BET surface area was measured at 647 m²/g (FIG. 13). BET measurements were also acquired for the alkyne Ugi-COF derivatives, featuring 463 m²/g and 827 m²/g surface areas, respectively, for hex-Ugi-COF—C≡C-[1.5](FIG. 14) and kag-Ugi-COF—C≡C-[1.0](FIG. 15). Despite a decrease in the surface area, the Ugi-modified materials still maintained their porous structure. This significant reduction in the surface area was due to several factors. The high density of the Ugi adducts in the bulk of the material reduces the COF pore volume due to its high steric occupancy. Furthermore, the rigid, fully conjugated backbone is converted to a partially flexible one due to the conversion of C═N bonds to the C—N bonds in the Ugi adduct. These observations were consistent with previously reported transformations of imine-based COFs to the amine-linked products (Lyle et al., “Multistep Solid-State Organic Synthesis of Carbamate-Linked Covalent Organic Frameworks,” J. Am. Chem. Soc., 141(28):11253-11258 (2019); Liu et al., “Covalent Organic Frameworks Linked by Amine Bonding for Concerted Electrochemical Reduction of CO₂,” Chem, 4(7):1696-1709 (2018); Grunenberg et al., “Amine-Linked Covalent Organic Frameworks as a Platform for Postsynthetic Structure Interconversion and Pore-Wall Modification,” J. Am. Chem. Soc., 143(9):3430-3438 (2021); Lu et al., “Asymmetric Hydrophosphonylation of Imines to Construct Highly Stable Covalent Organic Frameworks with Efficient Intrinsic Proton Conductivity,” J. Am. Chem. Soc., 144(22):9624-9633 (2022), which are hereby incorporated by reference in their entirety).

The N 1s X-ray photoelectron spectroscopy (XPS) profiles of the hex-COF and kag-COF before and after the Ugi reaction provided a handle for quantifying the efficiency of the multicomponent coupling reaction. After fitting the N 1s XPS spectra, both as-synthesized hex-COF (FIG. 8C, top) and kag-COF (FIG. 9C, top) contained a major peak at 398.5 eV, which corresponded to the imine nitrogen. Additional minor peaks were fitted for both COFs to account for the observed peak tailings. These fittings at 399.7 and 401.0 eV likely originate from the surface amines (yellow line) and a small amount of the imines oxidized to aromatic amides (orange line), respectively. This observation was attributed to the surface-sensitive nature of the XPS analysis (Korin et al., “Surface Analysis of Nanocomplexes by X-ray Photoelectron Spectroscopy (XPS),” ACS Biomater. Sci. Eng., 3(6):882-889 (2017), which is hereby incorporated by reference in its entirety). Due to the depth limit of the electron detection in the XPS, the results skew to provide a more accurate representation of the surface profile rather than the bulk of the material. However, the imine and amide signals attenuated significantly in the Ugi-modified materials. New XPS peaks observed at 399.6 eV in the spectra of the modified hex-Ugi-COF-OAc-[1.0](FIG. 8C, middle), hex-Ugi-COF—OAc-[1.5](FIG. 8C, bottom), and kag-Ugi-COF-OAc-[1.0](FIG. 9C, bottom) at 399.6 eV corroborated successful conversions of the imine nitrogen to Ugi amide nitrogen. The relative area under the fitting curves of the imine and amide nitrogen species in the Ugi-modified materials provided an approximate functionalization value of 70% for the hex-Ugi-COF-OAc-[1.0], 85% for the hex-Ugi-COF-OAc-[1.5], and 73% for the kag-Ugi-COF-OAc[1.0]. Although, XPS yields surface-weighted quantification.

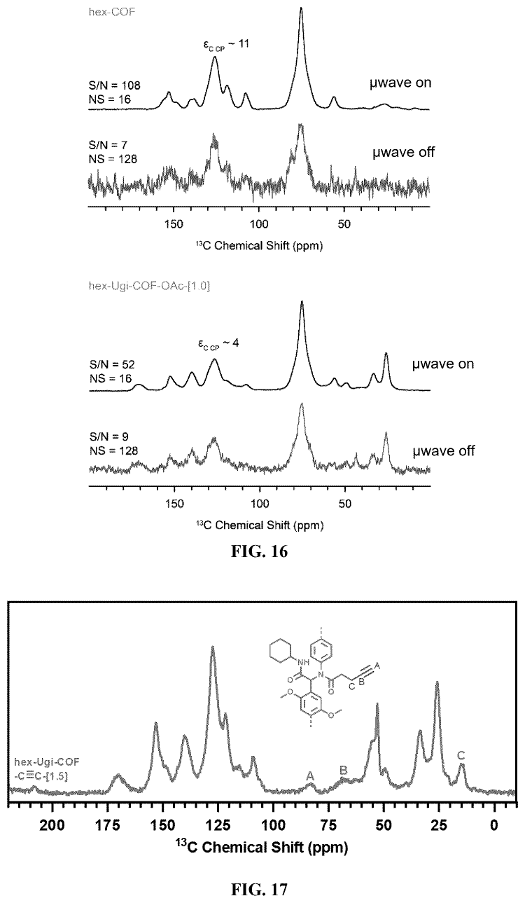

Additional confirmation of the imine nitrogen transformation to the Ugi amide nitrogen was obtained by using magic angle spinning (MAS) dynamic nuclear polarization (DNP) to enhance solid-state NMR sensitivity (Maly et al., “Dynamic Nuclear Polarization at High Magnetic Fields,” Chem. Phys., 128(5):052211 (2008); Ni et al., “High Frequency Dynamic Nuclear Polarization,” Acc. Chem. Res., 46(9):1933-1941 (2013), which are hereby incorporated by reference in their entirety) and enable ¹H→¹⁵N cross-polarization MAS (CPMAS) SSNMR experiments. The DNP-enhanced ¹⁵N CPMAS NMR spectra verified the nitrogen functional groups present in the frameworks before and after functionalization. DNP has been used previously to enhance the sensitivity of solid-state NMR experiments on a variety of porous materials such as silica (Lesage et al., “Surface Enhanced NMR Spectroscopy by Dynamic Nuclear Polarization,” J. Am. Chem. Soc., 132(44):15459-15461 (2010); Grüning et al., “Molecular-Level Characterization of the Structure and the Surface Chemistry of Periodic Mesoporous Organosilicates Using DNP-Surface Enhanced NMR Spectroscopy,” Phys. Chem. Chem. Phys., 15(32):13270-13274 (2013), which are hereby incorporated by reference in their entirety), MOF (Rossini et al., “Dynamic Nuclear Polarization Enhanced Solid-State NMR Spectroscopy of Functionalized Metal-Organic Frameworks,” Angew. Chem. Int. Ed., 51(1):123-127 (2012); Kobayashi et al., “DNP-Enhanced Ultrawideline Solid-State NMR Spectroscopy: Studies of Platinum in Metal-Organic Frameworks,” J. Phys. Chem. Lett., 7(13):2322-2327 (2016), which are hereby incorporated by reference in their entirety), COF (Cao et al., “Exploring Applications of Covalent Organic Frameworks: Homogeneous Reticulation of Radicals for Dynamic Nuclear Polarization,” J. Am. Chem. Soc., 140(22):6969-6977 (2018), which is hereby incorporated by reference in its entirety), polymers (Blanc et al., “Dynamic Nuclear Polarization NMR Spectroscopy Allows High-Throughput Characterization of Microporous Organic Polymers,” J. Am. Chem. Soc., 135(41):15290-15293 (2013), which is hereby incorporated by reference in its entirety, and zeolites (Wolf et al., “NMR Signatures of the Active Sites in Sn-β Zeolite,” Angew. Chem. Int. Ed., 53(38):10179-10183 (2014); Gunther et al., “Dynamic Nuclear Polarization NMR Enables the Analysis of Sn-Beta Zeolite Prepared with Natural Abundance ¹¹⁹Sn Precursors,” J. Am. Chem. Soc., 136(17):6219-6222 (2014), which are hereby incorporated by reference in their entirety). To perform DNP experiments on the COF materials, they were impregnated with a 16 mM solution of the nitroxide biradical TEKPOl (Zagdoun et al., “Large Molecular Weight Nitroxide Biradicals Providing Efficient Dynamic Nuclear Polarization at Temperatures up to 200 K,” J. Am. Chem. Soc., 135(34):12790-12797 (2013), which is hereby incorporated by reference in its entirety) dissolved in tetrachloroethane (Lesage et al., “Surface Enhanced NMR Spectroscopy by Dynamic Nuclear Polarization,” J. Am. Chem. Soc., 132(44):15459-15461 (2010); Zagdoun et al., “Non-Aqueous Solvents for DNP Surface Enhanced NMR Spectroscopy,” Chem Comm, 48(5):654-656 (2012), which are hereby incorporated by reference in their entirety). ¹H→¹³C CPMAS experiments performed with and without microwaves showed indirect ¹H CPMAS DNP enhancements (ε_{C cp}) of zz11 and 4 for the hex-COF and hex-Ugi-COF—OAc-[1.0], respectively. ¹H→¹³C CPMAS saturation recovery experiments showed that the ¹H DNP signal build-up times (T_{DNP}) were on the order of 5 s for the framework ¹H spins (FIG. 16). These DNP enhancements and the relatively short inter-scan delays were sufficient to permit the acquisition of natural abundance ¹H→¹⁵N CPMAS NMR spectra in ca. 11 hours for hex-COF and 19 hours for hex-Ugi-COF-OAc-[1.0].

The ¹⁵N CPMAS spectra were acquired with a 5 ms CP contact time to observe ¹⁵N NMR signals from both protonated and non-protonated nitrogen sites. The ¹H→¹⁵N CPMAS spectrum of the hex-COF shows intense NMR signals at −67 ppm, −243 ppm, −319 ppm, and −340 ppm that are assigned to imine, aromatic amide, amine, and ammonium (likely, from the acetate salt) nitrogen sites, respectively (FIG. 8D, top). These results are consistent with the XPS spectra, which indicate that there are at least three distinct nitrogen environments in the hex-COF. The DNP-enhanced ¹H→¹⁵N CPMAS are non-quantitative, which explains why the integrated intensity of the imine NMR signals is comparable to that of the amide and amine NMR signals. The non-quantitative signal intensities arise because the ¹H→¹⁵N CP efficiency is expected to be lower for the imine sites because imine nitrogen atoms are not protonated and are remote from nearby ¹H spins. The DNP enhancements may also be surface-weighted. Although these COF materials should have pores large enough to admit the TEKPol molecules, diffusion of the radicals is likely slow, and there will likely be higher DNP enhancements for the outer regions of the COF particles (Rossini et al., “Dynamic Nuclear Polarization Enhanced Solid-State NMR Spectroscopy of Functionalized Metal-Organic Frameworks,” Angew. Chem. Int. Ed., 51(1):123-127 (2012), which is hereby incorporated by reference in its entirety).

A gradient in DNP enhancements would likely preferentially enhance the intensity of amide and amine groups that are expected to be associated with incompletely condensed linkers present at the surface of the COF particles prior to Ugi functionalization. Regardless, the ¹H→¹⁵N CPMAS spectrum of hex-Ugi-COF-OAc-[1.0] shows that all imine signals are absent and that the most Intense ¹⁵N NMR signals are now present at amide chemical shifts (FIG. 8D). These observations are consistent with the conversion of most imine nitrogens into secondary and tertiary amides within the Ugi adducts. The NMR spectra are also consistent with XPS analysis which shows that a majority of imine sites have been converted to amides.

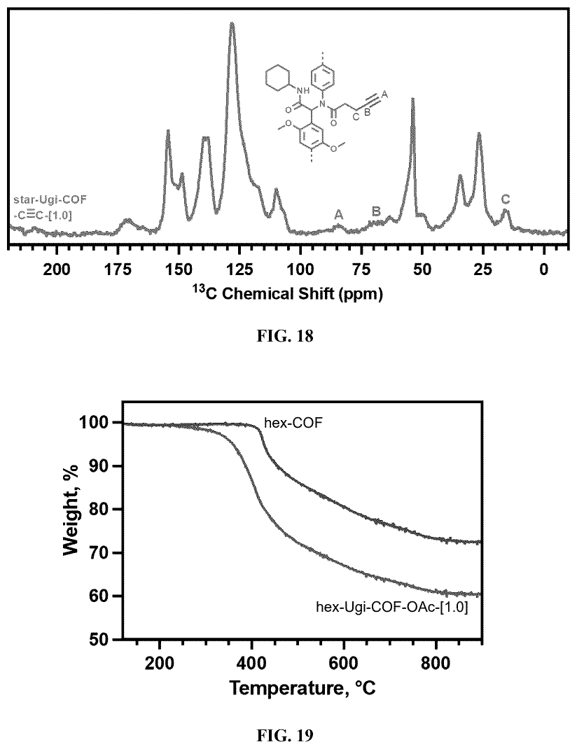

To verify the bulk functionalizations of the Ugi-COF materials, ¹³C CPMAS solid-state NMR spectroscopy was employed. Comparison of the ¹³C solution-state NMR spectrum of the model compound (FIG. 8E) with the ¹³C CPMAS solid-state NMR spectrum of the hex-Ugi-COF-OAc-[1.0](FIG. 8E) revealed an excellent match of all the diagnostic peaks, none of which were present in as-synthesized hex-COF (FIG. 8E). Specifically, carbonyl carbons at 170.7 ppm (A), benzyl carbon at 58.1 ppm (D), cyclohexyl carbon at 49.0 ppm (F), acetamide carbon at 32.2 ppm (G), and the cyclohexyl group of carbons at 29.7-17.1 ppm (H) were clearly identified in the ¹³C CPMAS NMR spectrum of the hex-Ugi-COF-OAc-[1.0]. In the case of kag-Ugi-COF-OAc-[1.0](FIG. 9F), the ¹³C CPMAS NMR spectrum also provided a match with the model compound (FIG. 9F), including the signals of carbonyl carbons at 170.0 ppm (A), benzyl carbon at 58.6 ppm (D), cyclohexyl carbon at 49.8 ppm (F), acetamide carbon at 33.8 ppm (G) and the cyclohexyl group carbons at 29.2-18.9 ppm (H). None of these signals were detected in as-synthesized kag-COF (FIG. 9F). In addition, ¹³C CPMAS NMR spectra for the alkyne Ugi-COF derivatives were acquired. Unlike other functional groups used, the terminal alkyne carbons produced diagnostic peaks in the ¹³C NMR, which served as clear evidence of successful Ugi-COF functionalization. The characteristic alkyne carbon signals were observed, as well as propargyl carbon signal in the hex-Ugi-COF≡C—C-[1.5](FIG. 17, 82.8 ppm (A), 67.8 ppm (B), 14.6 ppm (C)) and kag-Ugi-COF—C≡C-[1.0](FIG. 18, 83.5 ppm (A), 67.3 ppm (B), 14.9 ppm (C)).

In addition to confirming the incorporation of the Ugi adduct in both COFs, ¹³C CPMAS, NMR spectroscopy was used to more accurately quantify the efficiency of the Ugi modification in the hex-Ugi-COF-OAc-[1.0] and kag-Ugi-COF-OAc-[1.0]. ¹³C CPMAS solid-state NMR will be much more quantitative than DNP ¹⁵N CPMAS NMR spectra because only the ¹H→¹³C CP dynamics will affect the relative peak intensities. A 2 ms CP contact time was used to obtain all ¹³CPMAS spectra, which typically results in differences of ca. 15% or less in the integrated intensity relative to the atom ratio for all types of carbon atoms (Alemany et al., “Cross Polarization and Magic Angle Sample Spinning NMR Spectra of Model Organic Compounds. 1. Highly protonated molecules,” J. Am. Chem. Soc., 105(8):2133-2141 (1983), which is hereby incorporated by reference in its entirety). Signals at 152.5 ppm, 147.1 ppm, and 139.0 ppm in as-synthesized hex-COF were assigned to the aromatic carbon (B), imine carbon (I), and aromatic carbon (C), respectively (FIG. 8E). Since carbon (C) was not affected by the Ugi transformation, its signal was used as an internal standard to quantify the attenuation of the imine signal (I) in the hex-Ugi-COF-OAc-[1.0](FIG. 8E). Hence, the integration value of the signal (C) was set at 1.00 in the spectra of hex-COF and hex-Ugi-COF—OAc-[1.0]. In the case of hex-COF, the integration of the signals (B) and (I) together expectedly provided a value of 2.0 (1C+1C). However, integrations of the same signals in the hex-Ugi-COF-OAc-[1.0] provided a value of 1.30 (1C+0.3C). In other words, there were still ca. 30% (0.3C/1.0C) of the imine carbons present, while the remaining ca. 70% were converted to the Ugi adduct. This result is close to the functionalization value of 70% for the hex-Ugi-COF-OAc-[1.0], estimated by the XPS.

Thermogravimetric analysis (TGA) curves of hex-COF and hex-Ugi-COF-OAc-[1.0] are shown in FIG. 19.

The same analysis was applied to the ¹³C CPMAS solid-state NMR spectra of as-synthesized kag-COF and kag-Ugi-COF-OAc-[1.0]. Signals at 153.3 ppm, 147.5 ppm, 142.8-133.3 ppm were identified as aromatic carbon (B), imine carbon (I), and aromatic carbons (C), respectively, in kag-COF (FIG. 9F) and kag-Ugi-COF-OAc-[1.0](FIG. 9F). Carbon signals (C) were used as an internal standard and were set at the integration value of 2.0 (2C) in both spectra. The integration of the signals (B) and (I) together accounted for a value of 2.23 (1C+1.23C) in the case of kag-COF. For the kag-Ugi-COF—OAc-[1.0], the integration of the same signals (B) and (I) provided a value of 1.53 (1C.+0.53C), so there were ca. 43% (0.53/1.23) of the imine carbons present, while the rest ca. 57% were reacted to form the Ugi adduct. This result is a bit lower for the kag-Ugi-COF-OAc-[1.0] than the value of 73% estimated by the XPS. As discussed previously, the surface-sensitive nature of the XPS and DNP ¹⁵N CPMAS solid-state NMR likely skews the apparent degree of functionalization to a higher value. However, ¹³C CPMAS NMR spectra support the conversion of at least 50% to 60% of the imine bonds in the two COFs to the corresponding Ugi adducts in the case of the 1.0 M reaction conditions.

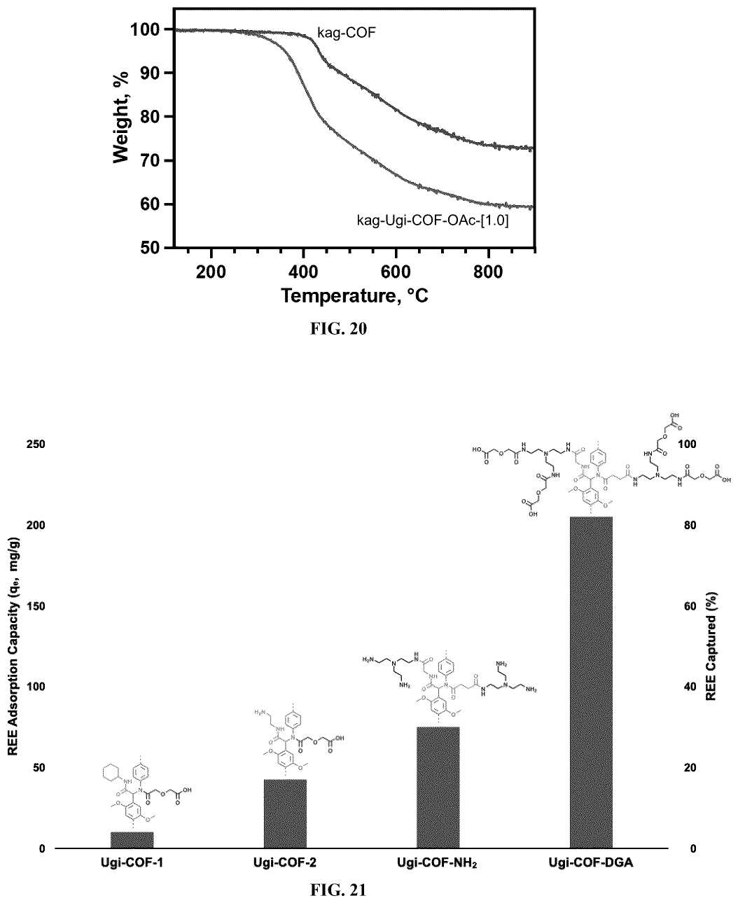

TGA curves of kag-COF and kag-Ugi-COF-OAc-[1.0] are shown in FIG. 20.

Note that the presented Ugi-COF materials were produced with a relatively high degree of functionalization for the purpose of efficient characterization. However, for most COF applications, the optimal active site loading varies between 20% to 30% (Xu et al., “Catalytic Covalent Organic Frameworks via Pore Surface Engineering,” Chem Comm, 50(11):1292-1294 (2014); Xu et al., “Stable, Crystalline, Porous, Covalent Organic Frameworks as a Platform for Chiral Organocatalysts,” Nat. Chem, 7(11):905-912 (2015), which are hereby incorporated by reference in their entirety). The strategy described herein meets this requirement and provides fine control over the functional group loading by tuning the concentration of the Ugi reaction conditions. Decreasing the functionalization degree of the Ugi-COFs would also help preserve most of its porous architecture.

In conclusion, a general strategy for the modification of imine-based COFs with functional group handles has been developed using the Ugi multicomponent reaction. The current facile approach to derivatize COFs with valuable functional group handles provides a gateway for the scientific community to advance the exploration of these crystalline porous materials. The potential of the operationally straightforward Ugi modifications in COFs, coupled with the versatile reactivity of the azide, alkyne, and vinyl functional handles, can result in the rapid production of highly customizable platforms that are poised to expand the applications of these materials in catalysis, separation, and molecular sensing.

### Example 4—Materials and Instrumentation for Examples 5-8

### Materials

Benzonitrile, mono-methyl succinate, tris(2-aminoethyl)amine, and neodymium(III) nitrate (Nd(NO₃)₃) were purchased from Millipore Sigma. Methanol, acetone, diethyl ether, and tetrahydrofuran were purchased from Fisher. Ethyl isocyanoacetate was purchased from Oakwood Chemical. Diglycolic anhydride was purchased from Combi-Blocks. 1,3,5-Tris(4-aminophenyl)benzene (TAPB) (Mancheno et al., “Introduction to Covalent Organic Frameworks: An Advanced Organic Chemistry Experiment,” J. Chem. Educ., 96(8):1745-1751 (2019), which is hereby incorporated by reference in its entirety) and 2,5-dimethoxyterephthalaldehyde (DMTP) (Kang et al., “Aggregated Structures of Two-Dimensional Covalent Organic Frameworks,” J. Am. Chem. Soc., 144(7):3192-3199 (2022), which is hereby incorporated by reference in its entirety) were synthesized according to previously reported protocols.

### Sonication

Sonication was performed with a Branson 2510 Ultrasonic Cleaner with a frequency of 40 kHz.

### Fourier-Transform Infrared (FTIR) Spectroscopy

Infrared spectra were collected using an Agilent Technologies Cary 600 Series FTIR Spectrometer equipped with a Diamond ATR accessory.

### Thermogravimetric Analysis (TGA)

TGA was measured on Netzsch STA449 F1 over the temperature range from 40 to 900° C. The analysis was carried out under an argon atmosphere with a heating rate of 10° C./min using an empty Al₂O₃ crucible as the reference.

### Powder X-Ray Diffraction (PXRD)

Powder X-ray diffraction patterns were collected using a Bruker D8-Advance diffractometer in parallel beam geometry employing Cu Kα (1.5418 Å) line-focused radiation at 40 kV/40 mA power and equipped with a position-sensitive detector. COF samples were loaded on zero background sample holders (MTI Corporation, ZeroSi32D20C1cavity10D) by dropping the powders from a metal spatula and leveling the sample. Samples were rotated at a rate of 15 rpm. Data were collected from 1.0° 20 to 30.0° 20 with 0.03° 20 per step (983 steps) and an exposure time of 7 s per step, for a total acquisition time of 126 min.

### X-Ray Photoelectron Spectra (XPS)

The XPS measurements were performed using a Kratos Amicus/ESCA 3400 instrument. The sample was irradiated with 240 W unmonochromated Mg Kα x-rays, and photoelectrons emitted at 0° from the surface normal were energy analyzed using a DuPont-type analyzer. The pass energy was set at 150 eV, and a Shirley baseline was removed from the spectra. CasaXPS was used to process raw data files.

### Inductively Coupled Plasma Optical Emission Spectroscopy (ICP-OES)

Agilent 5800 ICP-OES instrument was used to measure the rare-earth elements in the solution and consequent quantification of the adsorption/desorption capacities of the COFs. The intensity of 401.2 nm wavelength of Nd was used in the radial viewing mode for all the quantifications using a known calibration curve. A calibration curve consisting of 6 data points was constructed using 0-10 ppm Nd(NO₃)₃ solution in a 2% HNO₃ every time before analyzing the samples. All samples were diluted using a 2% HNO₃ solution before the analysis.

### Zeta Potential Measurements

The zeta potential measurements of the materials were performed in a Malvern Zetasizer NS using Smoluchowski equation and water as the dispersant. The refractive index of the COFs was assumed to be the same as the refractive index values of the carbon for all measurements.

### Scanning Transmission Electron Microscopy (STEM)

STEM images, high-angle annular dark-field scanning transmission electron microscopy (HAADF-STEM) images, and energy dispersive X-ray spectroscopy (EDS) were acquired on a FEI Titan Themis Probe Cs corrected scanning transmission electron microscope under 200 kV accelerating voltage. For experiments, 1 mg of the sample was dispersed in 2 mL ethanol, and then 50 μL mixture was dried on a TEM grid (Formvar/Carbon 200 mesh, Copper, TED PELLA, Inc.).

### Atomic Force Microscopy (AFM)

AFM images were acquired in AC mode using a CypherES (Oxford Instrument) using TESPA (Bruker) probes. All images were acquired in the open air. Samples were prepared by adding materials to 100% pure ethanol, followed by a 5-minute sonication and allowing the solution to rest for 5 minutes at room temperature. Next, a 5-μL droplet taken from the upper 10% of the sample solution was placed on a polished silicon surface (Ted Pella #16006), and ethanol was allowed to evaporate completely. AFM images were post-processed using the flattening (first order) technique. Height measurements were taken via cross-section within the Asylum software (v19.12.70).

### Gas Sorption Isotherms

Nitrogen physisorption experiments were conducted on a Micromeritics Tristar Analyzer using 80-100 mg samples in dried and tared analysis tubes equipped with filler rods and capped with a Transeal. Samples were pretreated at 100° C. for 6 hours under nitrogen gas flow. Each tube was weighed again to determine the mass of the activated sample and transferred to the analysis port of the instrument. UHP-grade (99.999% purity) N₂ was used for all sorption measurements. N₂ sorption isotherms were generated by incremental exposure to nitrogen up to 760 mmHg (1 atm) at 77 K (liquid N₂ bath). The Brunauer-Emmett-Teller (BET) method was utilized to calculate the specific surface areas.

### Conventional Solid-State Spinning NMR Spectroscopy

Room-temperature magic angle spinning (MAS)¹³C solid-state NMR experiments were performed with a 9.4 T widebore Bruker NMR spectrometer (ν₀(¹³C)=100.7 MHz) equipped with an Avance III HD console, and a Bruker 4 mm MAS probe configured in double resonance ¹H-¹³C mode. ¹³C solid-state NMR spectra were acquired with cross-polarization (CP) for signal enhancement (Pines et al., “Proton-Enhanced Nuclear Induction Spectroscopy. A Method for High Resolution NMR of Dilute Spins in Solids,” Chem. Phys., 56(4):1776-1777 (1972); Schaefer and Stejskal, “Carbon-13 Nuclear Magnetic Resonance of Polymers Spinning at the Magic Angle,” J. Am. Chem. Soc., 98(4):1031-1032 (1976), which are hereby incorporated by reference in their entirety). ¹H-¹³C CP matching conditions were optimized on an external standard of adamantane. CP experiments on COF materials used a 2 ms CP contact time. The magic angle spinning (MAS) frequency was 10 kHz. All ¹H CP spin-lock RF fields were linearly ramped from 90% to 100% amplitude (Metz et al., “Ramped-Amplitude Cross Polarization in Magic-Angle-Spinning NMR,” J. magn. reson., Ser. A, 110(2):219-227 (1994), which is hereby incorporated by reference in its entirety) to broaden the Hartman-Hahn match condition. The ¹H-¹³C CP experiments used spin lock pulses with RF fields of ca. 34 kHz and 44 kHz for ¹H and ¹³C, respectively. SPINAL-64 heteronuclear decoupling (Fung et al., “An Improved Broadband Decoupling Sequence for Liquid Crystals and Solids,” J. Magn. Reson., 142(1):97-101 (2000), which is hereby incorporated by reference in its entirety) with a ¹H RF field of 100 kHz was applied during the acquisition.

### Example 5—Synthetic Procedures

### Synthesis and Post-Synthetic Modifications of COFs

### Synthesis of Imine-COF

The imine-COF was prepared according to a previously reported single-crystalline protocol (Natraj et al., “Single-Crystalline Imine-Linked Two-Dimensional Covalent Organic Frameworks Separate Benzene and Cyclohexane Efficiently,” J. Am. Chem. Soc., 144(43):19813-19824 (2022), which is hereby incorporated by reference in its entirety), with some modifications. Benzonitrile (9.0 mL) was added to a scintillation vial (20 mL) containing benzoic acid (2.69 g, 22 mmol) and DMTP (56.0 mg, 0.28 mmol). The reaction vial was placed in a metal block and heated to 100° C. to fully dissolve the solids. Aniline (43 mg, 0.46 mmol) and TAPB (67.0 mg, 0.19 mmol) were separately dissolved in benzonitrile (1.0 mL and 2.0 mL, respectively), preheated to 100° C., and sequentially added to the reaction vial, with aniline modulator first, followed by the TAPB linker. The reaction was undisturbed for 10 min, maintaining the temperature at 100° C., followed by cooling to room temperature over the next 10 min. The solution was diluted with methanol, and the yellow precipitate was isolated by filtration, washed with copious amounts of methanol and acetone, and dried at 120° C. under reduced pressure (100 mTorr) for 24 hours, affording the product as a bright yellow powder (77 mg, 75% yield).

### Synthesis of Ugi-COF

In a 1-dram glass vial, the imine-COF (50 mg, 0.042 mmol, MW=1177.38 g/mol) was thoroughly mixed with mono-methyl succinate (101 mg, 0.76 mmol, 3 equiv×6 imine bonds per unit cell), followed by the addition of methanol (0.21 mL) and ethyl isocyanoacetate (86 mg, 0.76 mmol, 3 equiv×6 imine bonds per unit cell). Hence, the concentration of the acid and the isocyanide components was kept at 2.0 M each. The reaction was sealed with a Teflon cap and heated to 40° C. in a metal block for 12 hours without stirring. The progress of the reaction was monitored using the conjugated color-responsive structure of the COF: upon the initial addition of the acid, the material turned dark red (imine protonation), followed by a gradual change to light orange or yellow color (Ugi adduct formation), which indicated the reaction competition. The product was isolated by filtration, washed with methanol and diethyl ether, and dried at 120° C. under reduced pressure (100 mTorr) for 24 hours, affording Ugi-COF as an orange-yellow powder (99 mg, 88% yield).

### Synthesis of Ugi-COF—NH₂

In a 1-dram glass vial, the Ugi-COF (99 mg, 0.037 mmol, MW=2648.76 g/mol) was suspended in tris(2-aminoethyl)amine (0.5 mL, 3.57 mmol, 16 equiv×6 Ugi adducts per unit cell), sealed with a Teflon cap and heated to 60° C. in a metal block for 12 hours without stirring. The product was isolated by filtration, washed with methanol and diethyl ether, and dried at 120° C. under reduced pressure (100 mTorr) for 24 hours, affording Ugi-COF—NH₂ as a light coral powder (132 mg, 90% yield).

### Synthesis of Ugi-COF-DGA

In a 1-dram glass vial, the Ugi-COF—NH₂ (132 mg, 0.034 mmol, MW=3934.95 g/mol) was thoroughly mixed with diglycolic anhydride (0.473 mg, 4.08 mmol, 4 equiv×4 amine groups per Ugi adduct x 6 Ugi adducts per unit cell), sealed with a Teflon cap and heated to 100° C. in a metal block for 12 hours without stirring. The unreacted diglycolic anhydride was extracted thrice with hot tetrahydrofuran, and the product was isolated by filtration, washed with tetrahydrofuran, and dried at 120° C. under reduced pressure (100 mTorr) for 24 hours, affording Ugi-COF-DGA as a beige-brown powder (132 mg, 90% yield). Assuming quantitative functionalization at each step, the molecular weight of as-synthesized Ugi-COF-DGA was calculated to be 6630.47 g/mol. Hence, the effective loading of the diglycolic acid was determined at 3.62 mmol/g (1 g÷6630.47 g/mol×4 DGA groups per Ugi adduct×6 Ugi adducts per unit cell×1000 mmol/1 mol).

### Example 6—Adsorption Experiments and Characterization

### Ree Batch Adsorption Experiments

10 mg of the dried COF materials were precisely weighed and added in a 50 mL centrifuge tube. The COFs were then mixed with 50 ppm (50 mL) Nd(NO₃)₃ solution at a pH=6.2±0.3 and sonicated for 1 min. The mixture was then stirred at 500 rpm for 20 hours at 25° C. After that, the COFs were separated by centrifugation, and the supernatant was diluted using 2% HNO₃ and analyzed by ICP-OES to obtain the adsorption capacity (qₑ) of the materials.

$$Adsorption capacity ( {q}_{e} ) = ( {C}_{0} - {C}_{e} ) \times \frac{V}{m} mg / g$$

Here, C₀=initial Nd concentration, Cₑ=equilibrium concentration, V=total volume of the solution, m=mass of COF adsorbent.

Each experiment was done in triplicates to obtain the average qₑ value and the associated standard deviation. Unless stated otherwise, similar parameters were used for all studies. For the Ugi-COF-DGA, the following additional adsorption studies were conducted: pH-dependent Nd adsorption, Nd adsorption kinetics, and concentration dependent Nd adsorption.

### Ph-Dependent Nd Adsorption

Using similar conditions, the pH of the systems was adjusted from ca. 3.7 to ca. 6.2 by the addition of 0.1 M HNO₃ or 0.1 M NaOH solution, and the qₑ was measured to obtain a pH-dependent adsorption trend for the Ugi-COF-DGA. With the increase in the pH, the qₑ increased for the material.

### Nd Adsorption Kinetics

The contact time (contact time=sonication+stirring+centrifugation time) of the Ugi-COF-DGA with the Nd(NO₃)₃ solution was adjusted from 4 to 130 min to obtain the time-dependent adsorption trend. The material seemed to reach an adsorption equilibrium within ca. 70 min of contact time.

### Concentration Dependent Nd Adsorption

Keeping all other conditions the same, the initial Nd concentration was varied from 50 to 1000 ppm to obtain the concentration-dependent adsorption trend and the maximum adsorption capacity (qₘₐₓ) for the Ugi-COF-DGA. For these experiments, apart from analyzing the supernatant, the COF adsorbent was also analyzed to obtain accurate qₑ values for a higher concentration regime. After the separation of COF by centrifugation, the material was dried under vacuum (0.05 mBar). The dried material was precisely weighed and stirred with 2.0 M HNO₃ for 20 hours to desorb the adsorbed Nd. This solution was then analyzed by ICP-OES to obtain the concentration-dependent qₑvalues.

### Nd Concentration Dependent Zeta Potential Measurements

In a 15 mL centrifuge tube, ca. 2 mg of Ugi-COF-DGA was mixed with 5 mL of Nd(NO₃)₃ solutions of different concentrations from 0 to 200 ppm. The pH was adjusted at 6.2±0.3 by the addition of 0.1 M NaOH, and the mixture was sonicated for 1 min. Thereafter, the zeta potential was measured for each of the samples in triplicates to obtain the average zeta potential value for specific Nd concentrations.

Accessible Surface Area from Adsorption Experiments

The accessible surface area of the Ugi-COF-DGA material to the Nd ions in an aqueous solution was estimated. First, the mass of the Nd atoms adsorbed by the Ugi-COF-DGA was converted to mol of Nd:

$$662 mg Nd \div 144.242 g Nd / mol = 0.00459 mol Nd .$$

Next, given the radius of the hydrated Nd(III) ion (249 pm or 249×10⁻¹² m (Rudolph et al., “On the Hydration of the Rare Earth Ions in Aqueous Solution,” Journal of Solution Chemistry 49:316-331 (2020), which is hereby incorporated by reference in its entirety)), the area covered by each Nd atom was calculated as follows:

$${Nd}_{surface area} = 4 π \times {r}^{2} = 4 π \times {( 249 \times 1 {0}^{- 12} m )}^{2} .$$

Then, the total area occupied by 0.00459 mol Nd atoms was calculated according to the formula below:

$$Total Area = mol of Nd \times ( Avogadro ’ s constant ) \times [ {Nd}_{surface area} ] = 0.00459 mol Nd \times ( 6.023 \times {10}^{23} ) \times [ 4 π \times {( 249 \times {10}^{- 12} m )}^{2} ] = 2154 {m}^{2}$$

Therefore, the estimated accessible surface area of the Ugi-COF-DGA to Nd atoms was 2154 m²/g, which was in the same order of magnitude as the surface area of 2383 m²/g for the pristine imine-COF. Some loss in the accessible surface area can be attributed to the newly installed functionality of the DGA groups and/or waters of hydration surrounding the Nd ions during adsorption.

### Fitting of Nd Adsorption Isotherm Data

The experimental Nd isotherm data was fitted to the linear form of the Langmuir adsorption model using the following equation:

$$\frac{{C}_{e}}{{q}_{e}} = ( \frac{{C}_{e}}{{q}_{max}} ) + ( \frac{1}{{k}_{L} {q}_{max}} )$$

where Cₑ=equilibrium adsorption concentration, qₑ=corresponding adsorption capacity at that specific Cₑ value, qₘₐₓ=maximum adsorption capacity of the material, k_{L}=Langmuir constant. Upon fitting the experimental isotherm data to the Langmuir model, a good fit (R²=0.988) was obtained, and the predicted qₘₐₓ value from the fitting was 667 mg/g, which is within the range of the experimentally obtained maximum adsorption capacity of 663±29 mg/g.

### Ugi-COF-DGA Recyclability Studies

Ugi-COF-DGA (10 mg) was precisely weighed and added in a 50 mL centrifuge tube. The COFs were then stirred at 500 rpm in 50 mL of 50 ppm solution of Nd(NO₃)₃ at a pH=6.2±0.3 for 20 hours at 25° C. After that, the COFs were separated by centrifugation, and the supernatant was diluted using 2% HNO₃ and analyzed by ICP-OES to obtain the adsorption capacity (qₑ) of the materials. After the separation of COF by centrifugation, the material was dried under vacuum (0.05 mBar) at 25° C. The dried material was precisely weighed and stirred with 2.0 M HNO₃ for 20 hours at 25° C. to desorb Nd. This solution was then analyzed by ICP-OES to obtain the desorption capacity of the material. After the desorption treatment, the material was washed with DI H₂O twice and dried under vacuum (0.05 mBar) at 25° C. overnight (ca. 16 hours). The dried COF was then utilized for the next consecutive adsorption-desorption cycles, and similar treatments were done after each adsorption or desorption experiment. Each experiment was done at least in triplicates to obtain the average qₑ and the associated standard deviation for each cycle. Any adsorbent mass loss during recycling was addressed by adjusting the

$$“ \frac{m}{V} ”$$

ratio of the system.

### Fitting of Nd Adsorption Kinetics Data

The experimental Nd adsorption kinetics data was fitted to the linear form of the standard pseudo-first-order and pseudo-second-order models. More details on the models are provided below:

$$Linear Pseudo - First - Order Model : log ( {q}_{eq} - {q}_{e} ) = log ( {q}_{eq} ) - ( \frac{{k}_{1}}{2.303} ) \times t$$

$$Linear Pseudo - second - Order Model : \frac{t}{{q}_{e}} = ( \frac{1}{{k}_{2} {q}_{eq}^{2}} ) + ( \frac{t}{{q}_{eq}} )$$

where q_{eq}=adsorption capacity (qₑ) at the equilibrium, qₑ=adsorption capacity at a specific time point, k₁=pseudo-first-order rate constant, k₂=pseudo-second-order rate constant, and t=contact time. Upon fitting the data to both of these standard kinetics models, a better fit was obtained for the pseudo-2^{nd} order model (R²=0.989) over the pseudo-1^{st} order model (R²=0.966).

### Time-Dependent Nd Desorption Studies

Nd [symbol] Ugi-COF-DGA samples were prepared by soaking Ugi-COF-DGA in 50 mL of 50 ppm solution of Nd(NO₃)₃ at pH=6.2±0.3 for 2 hours while stirring at 500 rpm at 25° C. The material was separated by centrifugation, and the supernatant was quantified by ICP-OES. The average Nd adsorption capacity for this material was 204 mg/g. The Nd [symbol] Ugi-COF-DGA was then dried overnight under vacuum (0.05 mBar), suspended in 10 mL 2.0 M HNO₃ solution, and stirred at 500 rpm at 25° C. for different amounts of time (7 min to 240 min). Aliquots of 100 μL were taken out at each of the time points for quantification by ICP-OES to determine the desorption capacity of the material. Triplicate runs were done for each time point to determine the average desorption capacity and the standard deviation.

### Example 7—Nd-Dy Binary Mixture Adsorption by Ugi-COF-DGA

Nd and Dy are both critical materials with significant real-world applications. Recycled electronics and permanent magnet feeds can serve as potential cheap sources for Nd/Dy mixtures along with natural sources of these REEs. However, separating them can be challenging due to their similar physico-chemical properties. Therefore, the novel Ugi-COF-DGA materials were utilized to target this mixture in order to observe selective REE capture.

In a 50 mL plastic centrifuge tube, Nd-Dy binary mixture solution (50+50 ppm) was added. The solution was acidified by adding 400 μL of 0.1M HNO₃. Then, 10 mg Ugi-COF-DGA and a magnetic stir bar were added to this mixture. The centrifuge tube was then sealed and sonicated for 2-3 minutes to finely suspend the COF. The mixture was stirred at 500 rpm for 2 hours at room temperature (25° C.). After 2 hours, the mixture was centrifuged, and the supernatant was analyzed using ICP-OES to obtain the adsorption capacities of Nd and Dy by the COF.

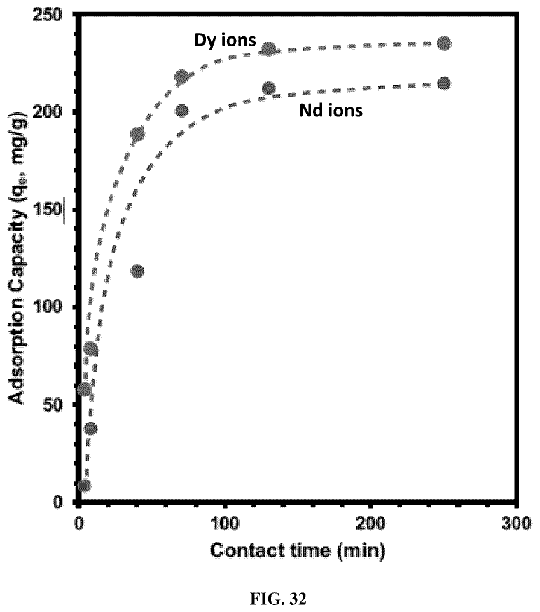

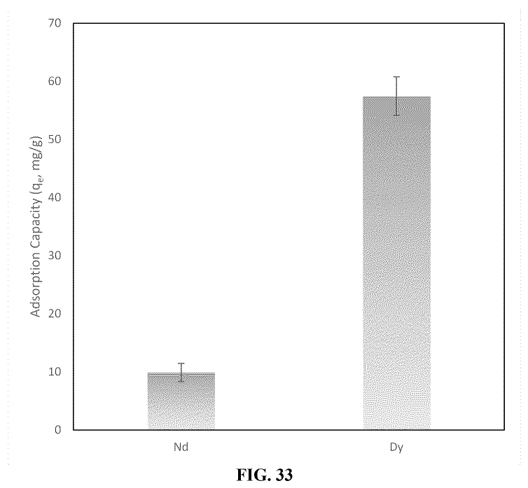

The procedure was repeated in triplicates to obtain the average adsorption capacities for Nd and Dy along with the standard deviations. The adsorption capacities of Nd and Dy were 9.9±1.6 mg/g and 57.4±3.3 mg/g, respectively (FIG. 33). It was observed that Dy was adsorbed approximately 6 times more compared to Nd. The higher adsorption of Dy might be correlated with the faster adsorption kinetics of Dy over Nd (FIG. 32).

### Example 8—Results and Discussion for Examples 4-7

### Design and Characterization of COFs

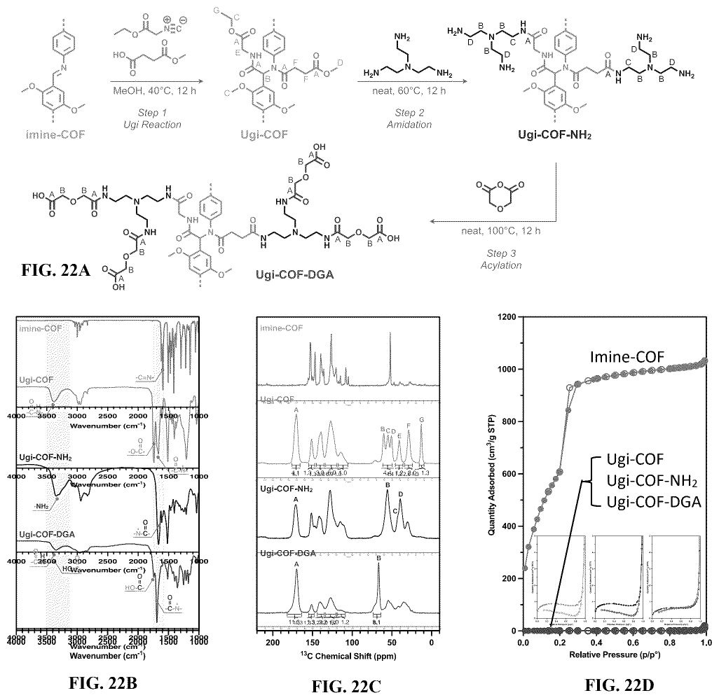

To incorporate the DGA motif for the first time in a COF, a chemically robust and highly crystalline COF (imine-COF) produced by the condensation of 1,3,5-tris(4-aminophenyl)benzene with 2,5-dimethoxyterephthalaldehyde via the single-crystalline synthetic protocol was selected (Natraj et al., “Single-Crystalline Imine-Linked Two-Dimensional Covalent Organic Frameworks Separate Benzene and Cyclohexane Efficiently,” J. Am. Chem. Soc., 144(43):19813-19824 (2022), which is hereby incorporated by reference in its entirety). To obtain the best material for REE adsorption, a series of post-synthetically modified COFs were rationally designed and tested, ultimately leading to the production of the Ugi-COF-DGA (FIG. 21). During the first step of the PSM, the imine-COF was treated with ethyl isocyanoacetate and mono-methyl succinate in methanol at 40° C. for 12 hours to install ester functional groups, yielding the Ugi-COF (FIG. 22A). Full conversion of the imine bonds to the corresponding Ugi bis-amide was critical to ensure the chemical stability of the material toward hydrolysis in extreme pH conditions, as well as the purity of the following synthetic transformations. During the second step, the ester groups of the Ugi-COF were amidated via the neat reaction with tris(2-aminoethyl)amine at 60° C. for 12 hours, providing the Ugi-COF—NH₂ with free amine groups. In the final step, Ugi-COF—NH₂ was reacted with neat diglycolic anhydride at 100° C. for 12 hours to afford Ugi-COF-DGA via the ring-opening acylation of the amines.

The successful synthetic transformation at each step was confirmed by Fourier-transform infrared (FTIR) spectroscopy and ¹³C cross-polarization magic angle spinning (CPMAS) solid-state nuclear magnetic resonance (SSNMR) spectroscopy. For the Ugi-COF, the FTIR spectrum revealed new amide frequencies at 1671 cm⁻¹ (C═O) and 3407 cm⁻¹ (N—H), as well as the ester frequency at 1743 cm⁻¹ (C═O) (FIG. 22B). In addition, all of the Ugi-COFs showed an absence of the imine frequency (C═N) that was observed at 1592 cm⁻¹ in the imine-COF precursor (FIG. 22B). ¹³C CPMAS SSNMR exhibited a new intense carbonyl carbon signal at 170.1 ppm (A, 4C), corresponding to the amide and ester functional groups, along with new aliphatic carbon signals at 60.4 ppm (B, 1C), 50.9 ppm (C, 1C), 40.9 ppm (D, 1C), 29.0 ppm (E, 1C), and 13.3 ppm (F, 2C) (FIG. 22C). The integration of the Ugi bis-amide signals with respect to the aromatic signals of the COF backbone (aromatic carbon signals in the 160 ppm to 110 ppm range were utilized as an internal standard, and their integration values were kept consistent throughout data analysis since no change was applied to the aromatic groups in any of the PSM steps) provided an excellent match to the proposed structure of the Ugi-COF.

The FTIR spectrum of the Ugi-COF—NH₂ displayed an intense amide frequency at 1670 cm⁻¹ (C═O), as well as a full disappearance of the ester frequency (C═O) (FIG. 22B). Furthermore, an intense broad frequency of the free amine groups at 3363 cm⁻¹ (N—H) was recorded. ¹³C CPMAS SS NMR revealed new aliphatic carbon signals at 55.7 ppm (B), 41.8 ppm (C), and 39.7 ppm (D), corroborating the successful incorporation of the amine functionalities (FIG. 22C). Carbon signals corresponding to the ester methoxy and ethoxy groups were not observed, suggesting the amidation reaction was highly efficient. The integration analysis was not performed for this material due to the overlap with the signals of the previously introduced functional groups.

In the case of the Ugi-COF-DGA, further amplification of the amide frequency at 1692 cm⁻¹ (C═O) was observed, as well as significant attenuation of the aliphatic amine frequency (N—H). A new frequency appeared at 1745 cm⁻¹ (C═O), corresponding to the DGA carboxylic acid functional group (FIG. 22B). In addition, a weak signal at 3350 cm⁻¹ was attributed to the carboxylic acid (O—H) and amide (N—H) functional groups. As for the ¹³C CPMAS SSNMR spectrum, two major peaks at 170.1 ppm (A, 12C) and 67.1 ppm (B, 8C) corresponding to the DGA functionality were detected (FIG. 22C). The integration of the DGA signals A and B, with respect to the COF backbone aromatic signals (in the 160 ppm to 110 ppm range, set in agreement with the Ugi-COF integration values), suggests near-quantitative conversion at each step, resulting in the loading of the DGA functional groups of ca. 3.62 mmol/g.

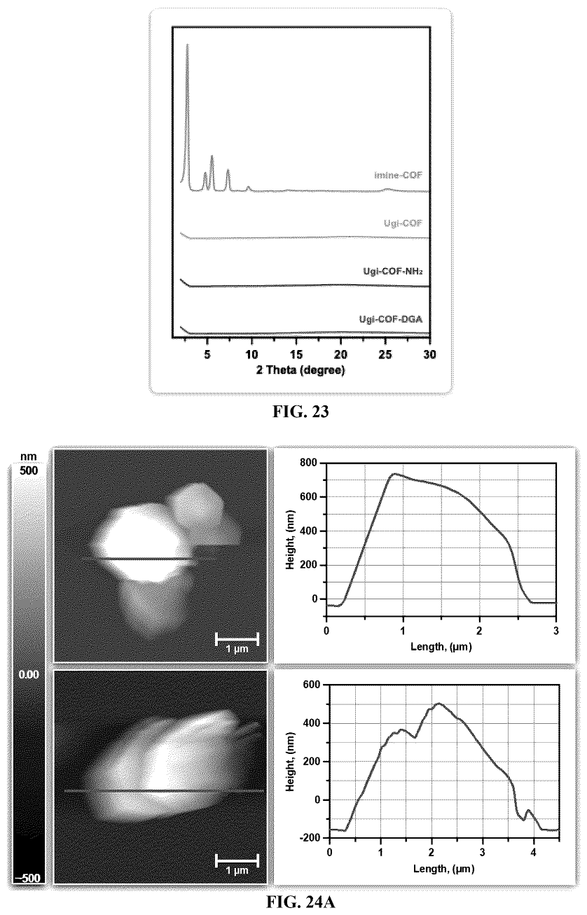

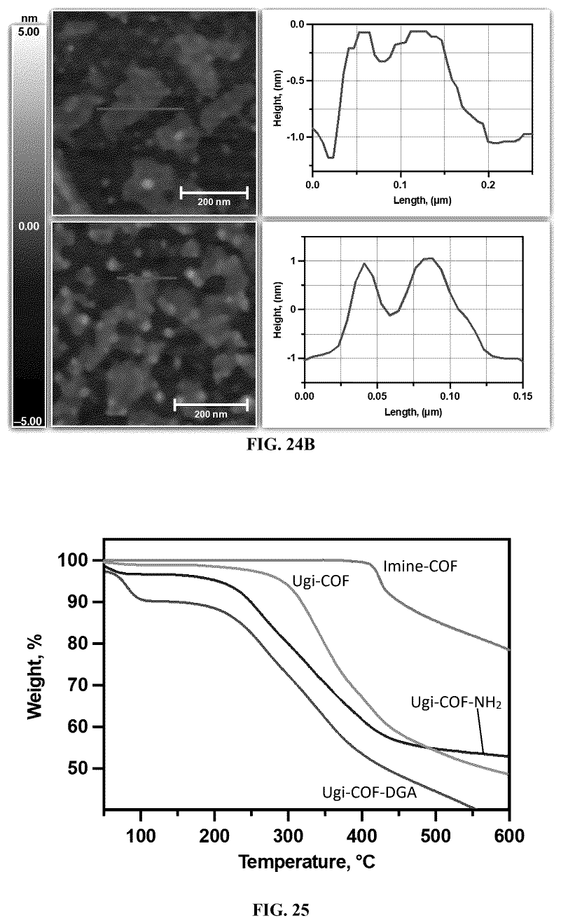

A complete loss of porosity of the Ugi-COFs in dry form was observed after the initial Ugi reaction on the imine-COF. In particular, the Brunauer-Emmett-Teller (BET) surface area of 2383 m²/g was recorded for the imine-COF, while only 3 m²/g was measured for the Ugi-COF and the Ugi-COF—NH₂, and 2 m²/g for the Ugi-COF-DGA (FIG. 22D). To account for such a dramatic loss in porosity, powder X-ray diffraction (PXRD) patterns of all four materials in dry form were collected. Through the Ugi modification, crystalline imine-COF was converted to amorphous Ugi-COF, which led to other materials produced in subsequent steps being amorphous as well (FIG. 23). This behavior is well documented in other two-dimensional (2D) COFs that were utilized in post-synthetic modifications on the imine linkage, where initially rigid, C═N-linked crystalline frameworks were effectively converted to flexible, C—N-linked materials, causing disruptions of the delicate π-π stacking between the layers and, as a result, low BET surface areas and PXRD-silent profiles (Lyle et al., “Multistep Solid-State Organic Synthesis of Carbamate-Linked Covalent Organic Frameworks,” J. Am. Chem. Soc., 141(28):11253-11258 (2019); Grunenberg et al., “Amine-Linked Covalent Organic Frameworks as a Platform for Postsynthetic Structure Interconversion and Pore-Wall Modification,” J. Am. Chem. Soc., 143(9):3430-3438 (2021); Zhang et al., “Construction of Flexible Amine-linked Covalent Organic Frameworks by Catalysis and Reduction of Formic Acid via the Eschweiler-Clarke Reaction,” Angew. Chem., Int. Ed., 60(22):12396-12405 (2021); Lyu et al., “Covalent Organic Frameworks for Carbon Dioxide Capture from Air,” J. Am. Chem. Soc., 144(28):12989-12995 (2022); Lu et al., “Asymmetric Hydrophosphonylation of Imines to Construct Highly Stable Covalent Organic Frameworks with Efficient Intrinsic Proton Conductivity,” J. Am. Chem. Soc., 144(22):9624-9633 (2022), which are hereby incorporated by reference in their entirety). This challenge was previously overcome in 3D COFs, where analogous post-synthetic transformations were shown to preserve the physical properties of the reticular structures, including high porosity and crystallinity (Liu et al., “Covalent Organic Frameworks Linked by Amine Bonding for Concerted Electrochemical Reduction of CO₂,” Chem, 4(7):1696-1709 (2018); Zhang et al. “Construction of Rigid Amine-Linked Three-Dimensional Covalent Organic Frameworks for Selectively Capturing Carbon Dioxide,” Chem Comm, 59(33):4911-4914 (2023); Zeppuhar et al., “Linkage Transformations in a Three-Dimensional Covalent Organic Framework for High-Capacity Adsorption of Perfluoroalkyl Substances,” ACS Appl. Mater. Interfaces, 15(45): 52622-52630 (2023), which are hereby incorporated by reference in their entirety). Different structural responses of the 2D and 3D systems to the increased degrees of rotational freedom may suggest an exfoliation mechanism of the 2D COFs induced by the formation of flexible linkages (Zhou et al., “Gradually Tuning the Flexibility of Two-Dimensional Covalent Organic Frameworks via Stepwise Structural Transformation and Their Flexibility-Dependent Properties,” Angew. Chem., Int. Ed., 62(38):e202305131 (2023), which is hereby incorporated by reference in its entirety). To confirm this, atomic force microscopy (AFM) imaging of as-synthesized imine-COF and Ugi-COF-DGA was performed. The imine-COF particles were found to be 500-700 nm high and several microns wide (FIG. 24A), while Ugi-COF-DGA particles were observed on the order of only 1-3 nm high and a tenth of a micron wide (FIG. 24B). Such a dramatic reduction in particle dimensions of more than 100-fold indicates exfoliation of the Ugi-COF-DGA in solution. The exfoliation is beneficial as it should enhance the diffusion of the REE ions inside the COF pores and promote better access to the binding sites, which could lead to improved adsorption capacity and faster adsorption/desorption kinetics. Therefore, despite the non-porous nature of generated materials (in dry form), Ugi-COFs possess improved chemical stability due to robust Ugi bis-amide linkages, as well as molecular functionality of the DGA groups for REE capture in aqueous solution. Furthermore, Ugi-COF derivatives exhibited thermal stability up to 200° C. by thermogravimetric analysis (FIG. 25), making these materials suitable for a wide range of REE extraction conditions.

### REE Adsorption Studies of COFs

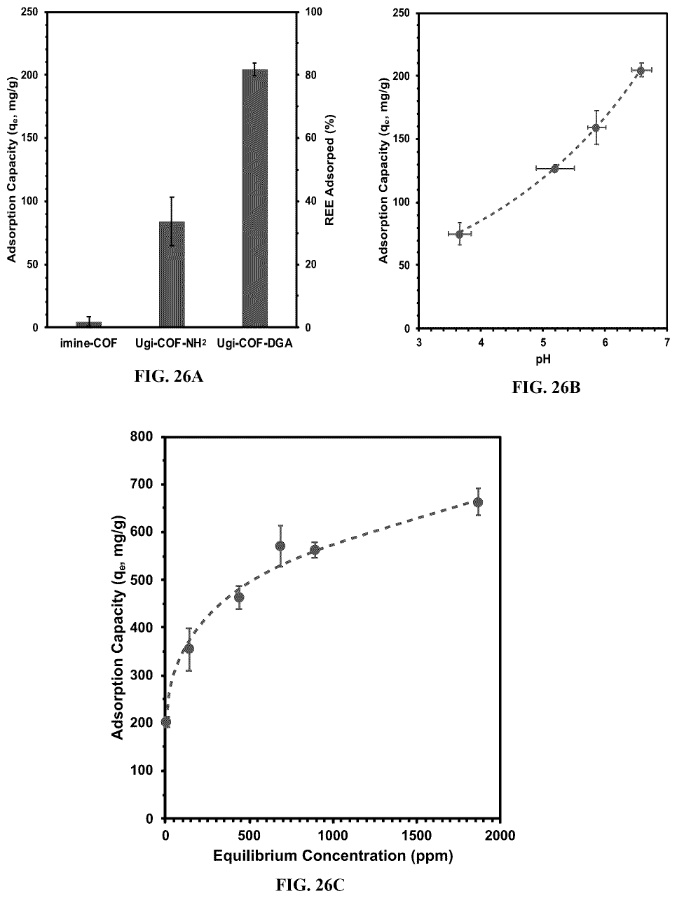

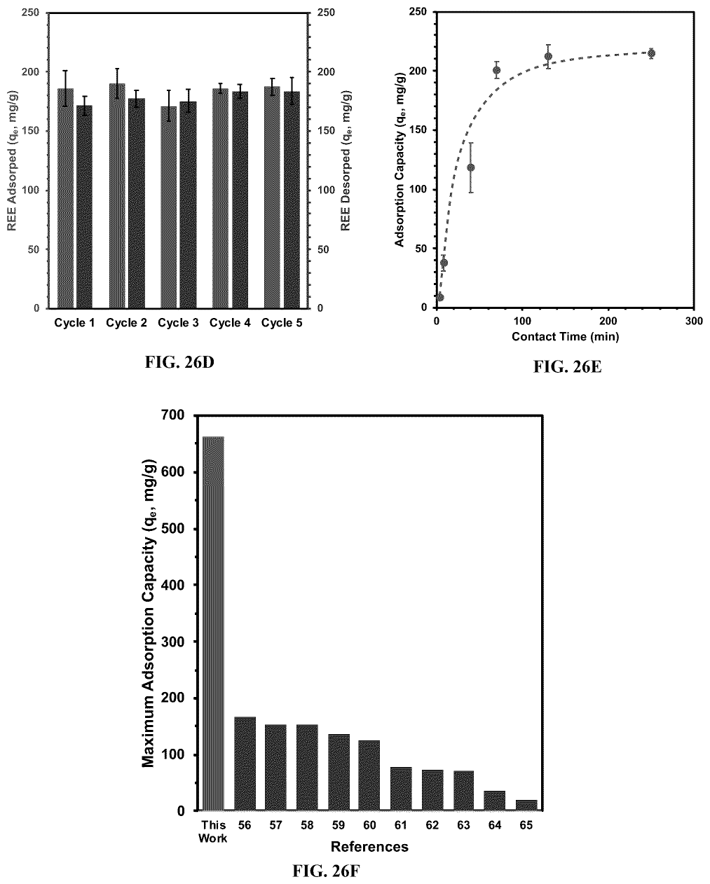

The performance of the COF materials for the REE adsorption was evaluated by mixing 10 mg of the adsorbent in 50 mL of 50 ppm solution of neodymium(III) nitrate (Nd(NO₃)₃) at a pH of ca. 6.4 with stirring at 25° C. for 20 hours. A preliminary assessment of the imine-COF revealed a minimal Nd adsorption capacity (qₑ) of ca. 5 mg of Nd per gram of COF (mg/g) despite the high porosity and crystallinity of the material (FIG. 26A). On the other hand, Ugi-COF—NH₂ showed a significant improvement in the qₑ value to ca. 84 mg/g, primarily due to the strong coordination ability of the aliphatic primary amine groups (Ramasamy et al., “Synthesis of Mesoporous and Microporous Amine and Non-Amine Functionalized Silica Gels for the Application of Rare Earth Elements (REE) Recovery from the Waste Water-Understanding the Role of pH, Temperature, Calcination and Mechanism in Light REE and Heavy REE Separation,” Chem. Eng. J., 322:56-65 (2017); Cristiani et al., “Capture and Release Mechanism of La Ions by New Polyamine-Based Organoclays: A Model System for Rare-Earths Recovery in Urban Mining Process,” J. Environ. Chem. Eng., 9(1):104730 (2021); Di Virgilio et al., “Analysis of the Adsorption-Release Isotherms of Pentaethylenehexamine-Modified Sorbents for Rare Earth Elements (Y, Nd, La),” Polymers, 14(23):5063 (2022), which are hereby incorporated by reference in their entirety). The addition of the DGA moiety into the Ugi-COF-DGA design provided the highest qₑ value of ca. 205 mg/g. Since this material exhibited the highest Nd capture capacity, further mechanistic studies of adsorption behavior were performed on the Ugi-COF-DGA.

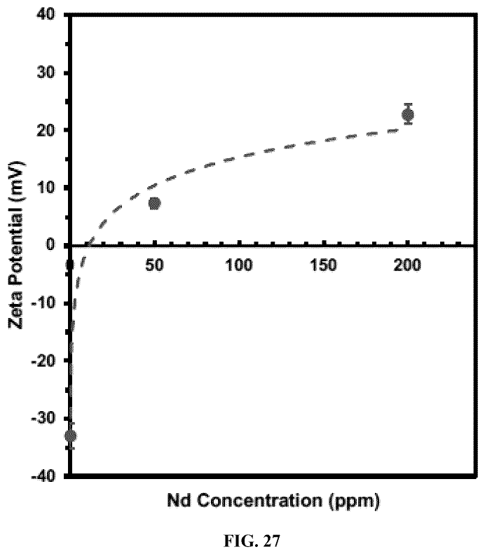

Favorable ionic interactions between negatively charged carboxylates of the DGA moiety and positively charged metal ions should bolster the adsorption capacity of the Ugi-COF-DGA. To verify this hypothesis, a pH-dependent Nd adsorption study was conducted for the Ugi-COF-DGA material (FIG. 26B). The adsorption capacity of the Ugi-COF-DGA improved from ca. 75 to 205 mg/g as the pH increased from ca. 3.7 to 6.4. Despite the observed enhancement of the qₑ value with a higher pH, a further increase in the pH was deemed unsuitable for adsorption testing due to the formation of metal hydroxides and their subsequent precipitation from the strongly alkaline solution. Hence, an optimal pH of ca. 6.4±0.2 was chosen for all other studies to ensure the best performance of the Ugi-COF-DGA. To provide additional insight into the DGA interaction with Nd ions, the zeta potential of the Ugi-COF-DGA was measured at a pH of ca. 6.2. The zeta potential provided a value of ca. −32.9 mV, indicating that the carboxylic groups of the DGA were substantially deprotonated (FIG. 27). When this material was exposed to the 50-ppm solution of Nd, the zeta potential value increased from −32.9 to +7.35 mV, confirming the interaction between the Nd ions and the DGA groups. Nd concentration of 200 ppm further increased the zeta potential to +22.7 mV, indicating a higher amount of metal ions interacting with the Ugi-COF-DGA.

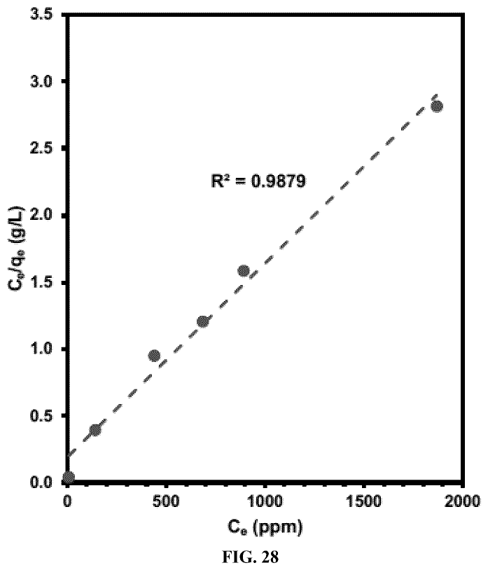

One of the critical properties distinguishing a promising adsorbent for REE capture is the material's ability to amplify the uptake of metal ions in a more concentrated solution of REEs. To evaluate the qₑ response of the Ugi-COF-DGA to higher REE concentrations, an isotherm plot was generated using Nd solutions of 50 mL with initial concentrations ranging from 50 to 2000 ppm, while keeping the amount of adsorbent constant at 10 mg. Ugi-COF-DGA demonstrated an increase in the qₑ value with a higher concentration of the Nd ions, varying from ca. 205 mg/g for the 50 ppm solution to 663±29 mg/g for the 2000 ppm solution (FIG. 26C). The qₑ value of 663 mg/g for Nd corresponds to ca. 4.5 mmol/g, which correlates to the maximum theoretical loading of the DGA groups in the Ugi-COF-DGA of 3.62 mmol/g. This comparison suggested that each DGA group was able to host at least one Nd atom in its bis-carbonyl moiety, while terminal carboxylates of the spatially adjacent groups could capture additional REE ions through cooperative coordination. The adsorption isotherm had a good fit to the Langmuir model (R²=0.988, FIG. 28), suggesting a monolayer adsorption mode, and the maximum predicted adsorption capacity qₘₐₓ of 667 mg/g, which is in the range of experimentally obtained value of 663±29 mg/g for the Ugi-COF-DGA. Based on the maximum adsorption capacity, the accessible surface area of the Ugi-COF-DGA to Nd atoms was estimated to be ca. 2154 m²/g (detailed calculations are provided in Example 6), which was close to the surface area of 2383 m²/g for the pristine imine-COF measured by N₂ adsorption. Lower Nd-based accessible surface area of the COF can be attributed to the newly installed DGA functionality, as well as waters of hydration surrounding the Nd ions during the adsorption process. This observation suggests that the binding sites of the Ugi-COF-DGA remain accessible to Nd ions in solution despite the non-porous properties of the material toward N₂ gas molecules.

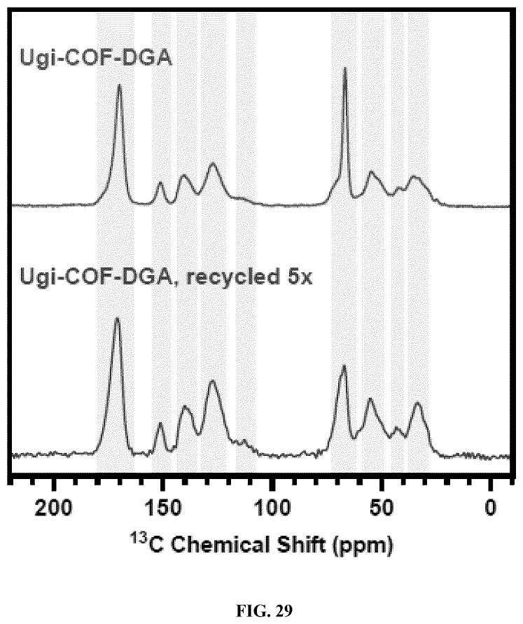

Consistently high performance and chemical stability of the material over multiple adsorption/desorption cycles are important factors for sustainable REE recovery. Hence, the recyclability of the Ugi-COF-DGA was evaluated over five adsorption/desorption cycles, and each data point was repeated thrice. Adsorption experiments were performed using 10 mg of the Ugi-COF-DGA in 50 mL of 50 ppm solution of Nd. After adsorption, Nd-loaded Ugi-COF-DGA (Nd [symbol] Ugi-COF-DGA) was isolated by filtration and suspended in 10 mL of 2.0 M nitric acid (HNO₃) at 25° C. for 20 hours with stirring, to ensure equilibrium was reached. Under these conditions, there was a near complete release of the adsorbed Nd (FIG. 26D). No significant deterioration of the adsorption-desorption capacity was detected for the Ugi-COF-DGA over multiple capture and release cycles, suggesting the COF adsorbent is chemically robust. To verify this, ¹³C CPMAS SSNMR of the Ugi-COF-DGA after five recycling runs was acquired and it was found that all characteristic peaks were present, and their chemical shifts were consistent with the as-synthesized Ugi-COF-DGA (FIG. 29). Such high molecular integrity and excellent recycling performance of the Ugi-COF-DGA in acidic aqueous conditions would not have been possible without converting hydrolyzable imine linkages of the imine-COF to stable bis-amide adducts. Ugi-COF-DGA is one of the most efficient, recyclable solid adsorbents for Nd capture reported to date (Table 1).

TABLE 1
Solid Adsorbents of Neodymium
$\text{Adsorption Capacit} y ( {q}_{e} ) = ( {C}_{0} - {C}_{e} ) \times \frac{V}{m} mg \cdot {g}^{- 1}$

| | Uptake Conditions | | | |
|---|---|---|---|---|
| | [C₀ (ppm), m (mg), V (mL), t (h), | qₑ | Multiple | |
| Material | pH, T (° C.)] | (mg · g⁻¹) | Recycles | Year |
| Cys-Chitosan | C₀ = 100, m = 50, V = 100, t = 4, pH | 17 | Yes | 2015 |
| Magnetic NPs^{a} | 5, T = 47° C. | | | |
| EDADGA-Silica^{b} | C₀ = 721, m = 50, V = 5, t = 24, pH | 17 | No | 2015 |
| | 1, T = RT | | | |
| Citric Acid | C₀ = 50, m = 2.5, V = 10, t = 0.5, pH | 41 | No | 2017 |
| Magnetite NPs^{c} | 7, T = RT | | | |
| Expanded | C₀ = 721, m = 700, V = 50, t = 6, pH | 49 | No | 2021 |
| Vermiculite^{d} | 3.3, T = RT | | | |
| L-cysteine | C₀ = 50, m = 2.5, V = 10, t = 0.5, pH | 86 | No | 2017 |
| magnetite NPs^{c} | 7, T = RT | | | |
| DGA-Polystyrene^{e} | C₀ = 180, m = 7.5, V = 10, t = 1, pH | 144 | No | 2020 |
| | 6, T = RT | | | |
| ZIF-8-COOH^{f} | C₀ = 500, m = 10, V = 10, t = 4, pH | 175 | Yes | 2021 |
| | 6, T = RT | | | |
| Calcium Alginate | C₀ = 606, m = 40, V = 25, t = 24, pH | 238 | Yes | 2014 |
| Polyglutamic Acid | 3.6, T = RT | | | |
| Gels^{g} | | | | |
| Anionic | C₀ = 1000, m = 3.5, V = 7, t = 0.09, | 264 | No | 2022 |
| Nanocellulose^{h} | pH 5.7, T = RT | | | |
| Calixarene | C₀ = 1120, m = 1, V = 5, t = 4, pH 7, | 312 | No | 2016 |
| Graphene Oxideⁱ | T = RT | | | |
| Phosphite-Porous | C₀ = 500, m = 10, V = 10, t = 4, pH | 321 | Yes | 2019 |
| Organic Polymer^{j} | 5, T = RT | | | |
| Ugi-COF-DGA | C₀ = 1000, m = 10, V = 50, t = 20, | 663 | Yes | 2024 |
| (this work) | pH 6.2, T = RT | | | |
| Carboxymethyl | C₀ = 500, m = 2, V = 5, t = 20, pH 5, | 661 | Yes | 2022 |
| Cellulose | T = RT | | | |
| Graphene Oxide^{k} | | | | |
| N-Enriched | C₀ = 1000, m = 30, V = 100, t = 6, | 840 | No | 2020 |
| Benzimidazole- | pH 6.4, T = RT | | | |
| Linked Polymeric | | | | |
| Network^{l} | | | | |

^{a}Galhoum et al., “Cysteine-Functionalized Chitosan Magnetic Nano-Based Particles for the Recovery of Light and Heavy Rare Earth Metals: Uptake Kinetics and Sorption Isotherms,” Nanomaterials, 5(1):154-179 (2015), which is hereby incorporated by reference in its entirety.
^{b}Ogata et al., “Adsorption Behavior of Rare Earth Elements on Silica Gel Modified with Diglycol Amic Acid,” Hydrometallurgy, 152:178-182 (2015), which is hereby incorporated by reference in its entirety.
^{c}Ashour et al., “Selective Separation of Rare Earth Ions from Aqueous Solution Using Functionalized Magnetite Nanoparticles: Kinetic and Thermodynamic Studies,” Chem. Eng. J., 327:286-296 (2017), which is hereby incorporated by reference in its entirety.
^{d}Brião et al., “Efficient and Selective Adsorption of Neodymium on Expanded Vermiculite,” Ind. Eng. Chem. Res., 60(13):4962-4974 (2021), which is hereby incorporated by reference in its entirety.
^{e}Pereao et al., “Adsorption of Ce³⁺ and Nd³⁺ by Diglycolic Acid Functionalised Electrospun Polystyrene Nanofiber from Aqueous Solution,” Sep. Purif. Technol., 233:116059 (2020), which is hereby incorporated by reference in its entirety.
^{f}Ahmed et al., “Aqueous Nd³⁺ Capture Using a Carboxyl-Functionalized Porous Carbon Derived from ZIF-8,” J. Colloid Interface Sci., 594:702-712 (2021), which is hereby incorporated by reference in its entirety.
^{g}Wang et al., ″Adsorption of Rare Earths (III) by Calcium Alginate-Poly Glutamic acid Hybrid Gels,” J. Chem. Technol. Biotechnol., 89(7):969-977 (2014), which is hereby incorporated by reference in its entirety.
^{h}Wamea et al., “Nanoengineering Cellulose for the Selective Removal of Neodymium: Towards Sustainable Rare Earth Element Recovery,” Chem. Eng. J., 428:131086 (2022), which is hereby incorporated by reference in its entirety.
ⁱZhang et al., “Calixarene-Functionalized Graphene Oxide Composites for Adsorption of Neodymium Ions from the Aqueous Phase,” RSC Adv., 6(36):30384-30394 (2016), which is hereby incorporated by reference in its entirety.
^{j}Ravi et al., “Porous Covalent Organic Polymers Comprising a Phosphite Skeleton for Aqueous Nd(III) Capture,” ACS Appl. Mater. Interfaces, 11(12): 11488-11497 (2019), which is hereby incorporated by reference in its entirety.
^{k}Abd-Elhamid et al., “Graphene Oxide Modified with Carboxymethyl Cellulose for High Adsorption Capacities towards Nd(III) and Ce(III) from Aqueous Solutions,” Cellulose, 29:9831-9846 (2022), which is hereby incorporated by reference in its entirety.
^{l}Maruthapandi et al., “Nitrogen-Enriched Porous Benzimidazole-Linked Polymeric Network for the Adsorption of La (III), Ce (III), and Nd (III),” J. Phys. Chem. C, 124:6206-6214 (2020), which is hereby incorporated by reference in its entirety.

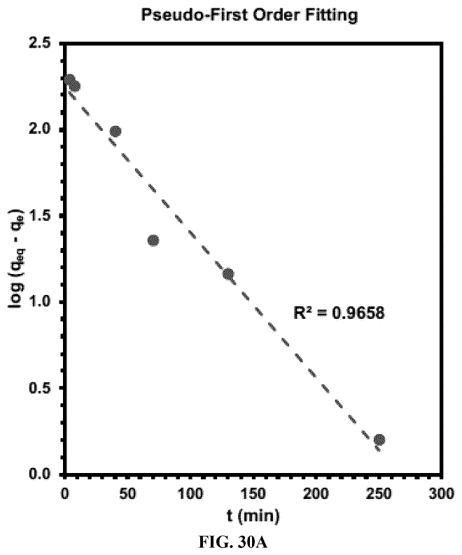

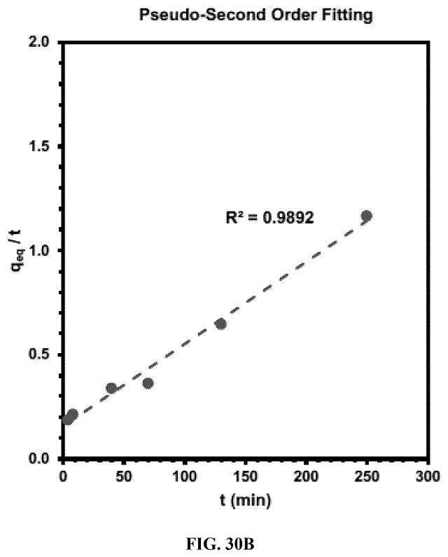

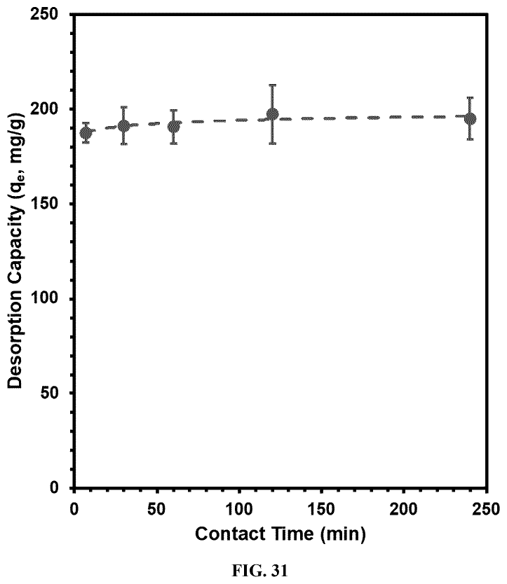

Fast adsorption kinetics is another attractive feature of a solid adsorbent for practical applications. Adsorption kinetics of the Ugi-COF-DGA were assessed by varying the contact time of 10 mg of COF material with 50 mL of 50 ppm solution of Nd. Ugi-COF-DGA reached its equilibrium qₑ value within 70 minutes of contact time (FIG. 26E). Time-dependent adsorption data was fitted to both the pseudo-first-order and pseudo-second-order kinetic models (FIGS. 30A-30B). The pseudo-second-order model provided a better fit (R²=0.989, for the experimental data than the first-order model (R²=0.965), suggesting that chemisorption, i.e., coordination between the DGA groups and the Nd ions was the main driving force behind the adsorption process. The Nd desorption kinetics of Ugi-COF-DGA was also studied. Samples of the Nd [symbol] Ugi-COF-DGA were thoroughly washed with deionized water to remove excess REEs, dried in vacuo, and subjected to time-dependent desorption studies in 2.0 M HNO₃. The release of the REE ions by Nd [symbol] Ugi-COF-DGA was almost immediate (FIG. 31). Such fast and efficient adsorption and desorption performance of the Ugi-COF-DGA makes it a promising material for application in flow separation. Interestingly, slightly faster adsorption kinetics were observed when Nd was substituted for Dy under otherwise identical experimental conditions (FIG. 32). This result showcases the ability of the Ugi-COF-DGA to adsorb other industrially critical REE ions with equally high efficiency, as well as provides an opportunity for kinetics-driven selectivity.

To assess the effect of the COF architecture as a solid support for the DGA ligand, the highest qₑ value of the Ugi-COF-DGA was compared with the maximum adsorption capacities reported for other DGA-functionalized solid materials (Florek et al., “Selective Recovery of Rare Earth Elements Using Chelating Ligands Grafted on Mesoporous Surfaces,” RSC Adv., 5(126):103782-103789 (2015), Pereao et al., “Adsorption of Ce³⁺ and Nd³⁺ by Diglycolic Acid Functionalised Electrospun Polystyrene Nanofiber from Aqueous Solution,” Sep. Purif Technol., 233:116059 (2020); Arrambide et al., “Extraction and Recovery of Rare Earths by Chelating Phenolic Copolymers Bearing Diglycolamic Acid or Diglycolamide Moieties,” React. Funct. Polym., 142:147-158 (2019); Pereao et al., “Synthesis and Characterisation of Diglycolic Acid Functionalised Polyethylene Terephthalate Nanofibers for Rare Earth Elements Recovery,” J. Environ. Chem. Eng., 9(5):105902 (2021); Li et al., “Selective Extraction and Column Separation for 16 Kinds of Rare Earth Element Ions by Using N, N-dioctyl Diglycolacid Grafted Silica Gel Particles as the Stationary Phase,” J. Chromatogr. A, 1627:461393 (2020); Bai et al., “Preparation of Elastic Diglycolamic-Acid Modified Chitosan Sponges and Their Application to Recycling of Rare-Earth from Waste Phosphor Powder,” Carbohydr. Polym., 190:255-261 (2018); Hamada et al., “Poly(vinyl diglycolic acid ester)-Grafted Polyethylene/Polypropylene Fiber Adsorbent for Selective Recovery of Samarium,” ACS Appl. Polym. Mater., 4(3):1846-1854 (2022); Momen et al., “Extraction Chromatographic Materials for Clean Hydrometallurgical Separation of Rare-Earth Elements Using Diglycolamide Extractants,” Ind. Eng. Chem. Res., 58(43):20081-20089 (2019); Ogata et al., “Immobilization of Diglycol Amic Acid on Silica Gel for Selective Recovery of Rare Earth Elements,” Chem. Lett., 43(9):1414-1416 (2014); Wang et al., “Preparation of Pyrrolidinyl Diglycolamide Bonded Silica Particles and its Rare Earth Separation Properties,” J. Chromatogr. A, 1681:46339 (2022), which are hereby incorporated by reference in their entirety). The newly developed Ugi-COF-DGA offers the highest adsorption capacity, the q_{c} value four times higher than the next best-reported DGA-modified supports (FIG. 26F). Furthermore, it exhibited one of the highest reported qₑ values for Nd adsorption to date (Table 1). In this context, despite the N-enriched benzimidazole-linked polymeric network having a higher adsorption capacity of 840 mg/g (Maruthapandi et al., “Nitrogen-Enriched Porous Benzimidazole-Linked Polymeric Network for the Adsorption of La (III), Ce (III), and Nd (III),” J. Phys. Chem. C, 124(11):6206-6214 (2020), which is hereby incorporated by reference in its entirety), recyclability was not demonstrated for this material, arguably making Ugi-COF-DGA the best recyclable sorbent for Nd capture.

[symbol]

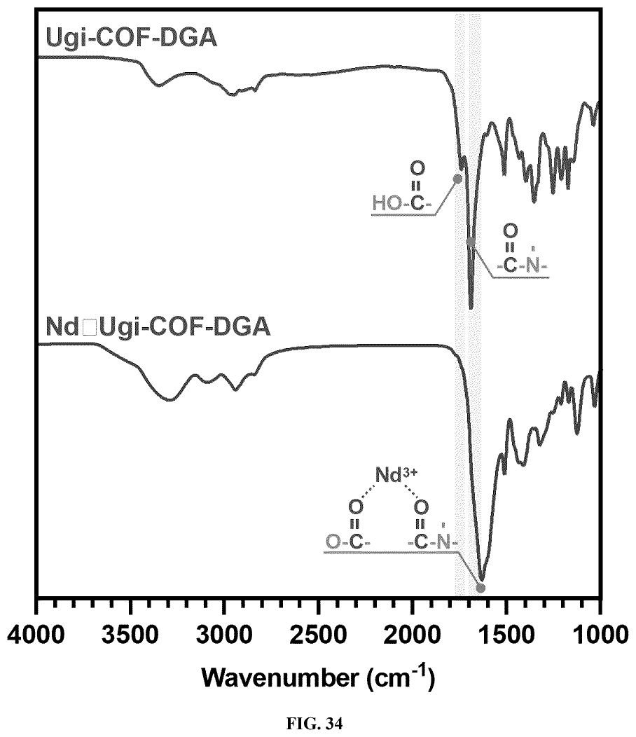

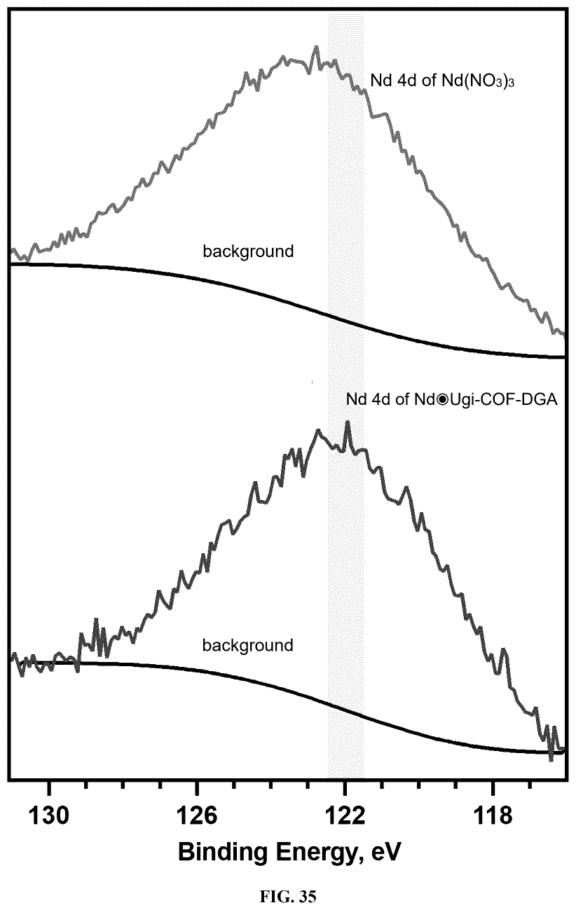

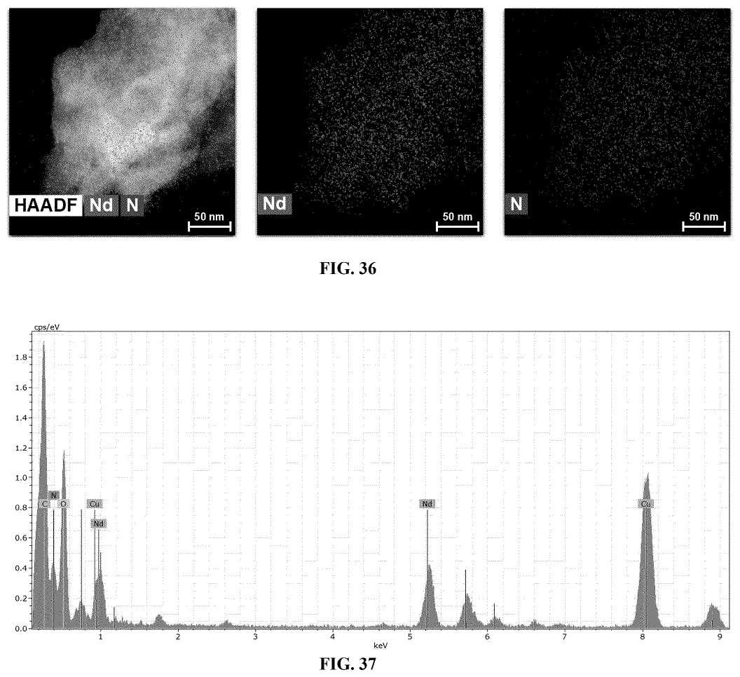

To verify the presence of Nd on the solid adsorbent, the Nd [symbol] gi-COF-DGA sample was recovered from the 1000 ppm Nd adsorption experiment, washed thoroughly with deionized water to remove excess REEs, dried in vacuo, and characterized. The FTIR spectrum of the Nd [symbol] Ugi-COF-DGA exhibited apparent shifts of the carboxylic acid and amide carbonyl stretches to a lower frequency at 1500 cm⁻¹, suggesting the weakening of the C═O bonds due to coordination to Nd (FIG. 34) which is consistent with the mechanistic studies of the DGA coordination to the REEs through the chelation to the carbonyl groups (Ogata et al., “Adsorption Mechanism of Rare Earth Elements by Adsorbents with Diglycolamic Acid Ligands,” Hydrometallurgy, 163:156-160 (2016); Matloka et al., “Highly efficient binding of trivalent f-elements from acidic media with a C3-symmetric tripodal ligand containing diglycolamide arms,” Dalton Transactions, 23:3719-3721 (2005); which are hereby incorporated by reference in their entirety). Additionally, previously reported single-crystal structures of lanthanide-DGA complexes further support the hypothesis of Nd binding through the carbonyl groups of the ligand (Ibrahim et al., “Selective Extraction of Light Lanthanides(III) by N,N-Di(2-ethylhexyl)-diglycolamic Acid: A Comparative Study with N,N-Dimethyl-diglycolamic Acid as a Chelator in Aqueous Solutions,” ACS Omega, 4:20797-20806 (2019); Peng et al., “Theoretical elucidation of rare earth extraction and separation by diglycolamides from crystal structures and DFT simulations,” J. Rare Earths, 39:858-865 (2021) which are hereby incorporated by reference in their entirety). Nd 4d X-ray photoelectron spectroscopy (XPS) analysis of the Nd [symbol] Ugi-COF-DGA showed the presence of the Nd peak at 122 eV, which appeared at a lower binding energy than the Nd(NO₃)₃ salt peak at 123 eV (FIG. 35). This negative shift of ca. 1 eV in the Nd XPS suggests the coordination interaction between the electron-donating DGA groups and the electron-accepting Nd ions, which is also congruent with FTIR observations. Importantly, energy dispersive X-ray spectroscopy (EDS) mapping of the Nd [symbol] Ugi-COF-DGA revealed a homogeneous distribution of the N and Nd atoms in the sample without apparent clustering of the metal ions, suggesting their successful adsorption by the COF material rather than precipitation from the solution (FIGS. 36 and 37).

In conclusion, it was demonstrated that the Ugi PSM strategy could be used to transform an imine-based COF into a readily customizable platform for efficient and recyclable adsorption of REEs. The novel Ugi-COF-DGA displayed the highest adsorption capacity among other DGA-functionalized solid supports, can be recycled at least five times without compromising the performance of the material, and had fast adsorption and desorption kinetics. Future studies will be focused on optimizing the Ugi-COFs reticular and molecular design to achieve selective REE capture and integrating these materials into devices for capture or separation of REE ions from solution. This work could serve as a general strategy for the expansion of the functional diversity in imine-based COFs and inspire further exploration of these materials as promising adsorbents for efficient and recyclable capture of the REEs.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A covalent organic framework comprising repeating units of a moiety comprising Formula (I): 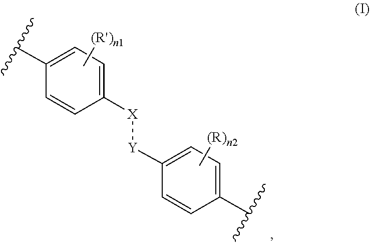
wherein
--- is a single or a double bond;
X is independently selected at each occurrence thereof from N or N(R³), wherein at least one X is N(R³);
Y is independently selected at each occurrence thereof from C(R¹)(R²) or C(R¹);
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R¹ is H;
R² is optional and, if present, is —C(O)NHR⁴;
R³ is —C(O)R⁵;
R⁴ is independently selected at each occurrence thereof from the group consisting of cycloalkyl, —(CH₂)ₘC(O)OC₁₋₆ alkyl, and —(CH₂)ₘC(O)NH(CH₂)ₖNH₂, wherein —(CH₂)ₘC(O)NH(CH₂)ₖNH₂ can be optionally substituted 1 to 3 times with R⁷;
R⁵ is selected from the group consisting of C₁₋₁₂ alkyl, C₂₋₁₂ alkenyl, or C₂₋₁₂ alkynyl, wherein C₁₋₁₂ alkyl can be optionally substituted 1 to 3 times with R⁶;
R⁶ is independently selected at each occurrence thereof from N₃, —C(O)OC₁₋₆ alkyl, —C(O)NH(CH₂)ₖNH₂, wherein —C(O)NH(CH₂)ₖNH₂ can be optionally substituted 1 to 3 times with R⁷;
R⁷ is —(CH₂)ₖ₁NH₂ optionally substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5, or 6; and
k₇ is 1, 2, 3, 4, 5, or 6.

2. The covalent organic framework according to claim 1, wherein m is 1, k is 2, k₁ is 2, k₂ is 1, and k₃ is 1.

3. The covalent organic framework according to claim 1, wherein R′ is H.

4. The covalent organic framework according to claim 1, wherein the repeating units of a moiety comprise Formula (I′):

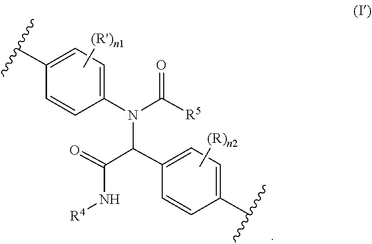

5. The covalent organic framework according to claim 1, wherein the repeating units of a moiety comprise a structure selected from the group consisting of:

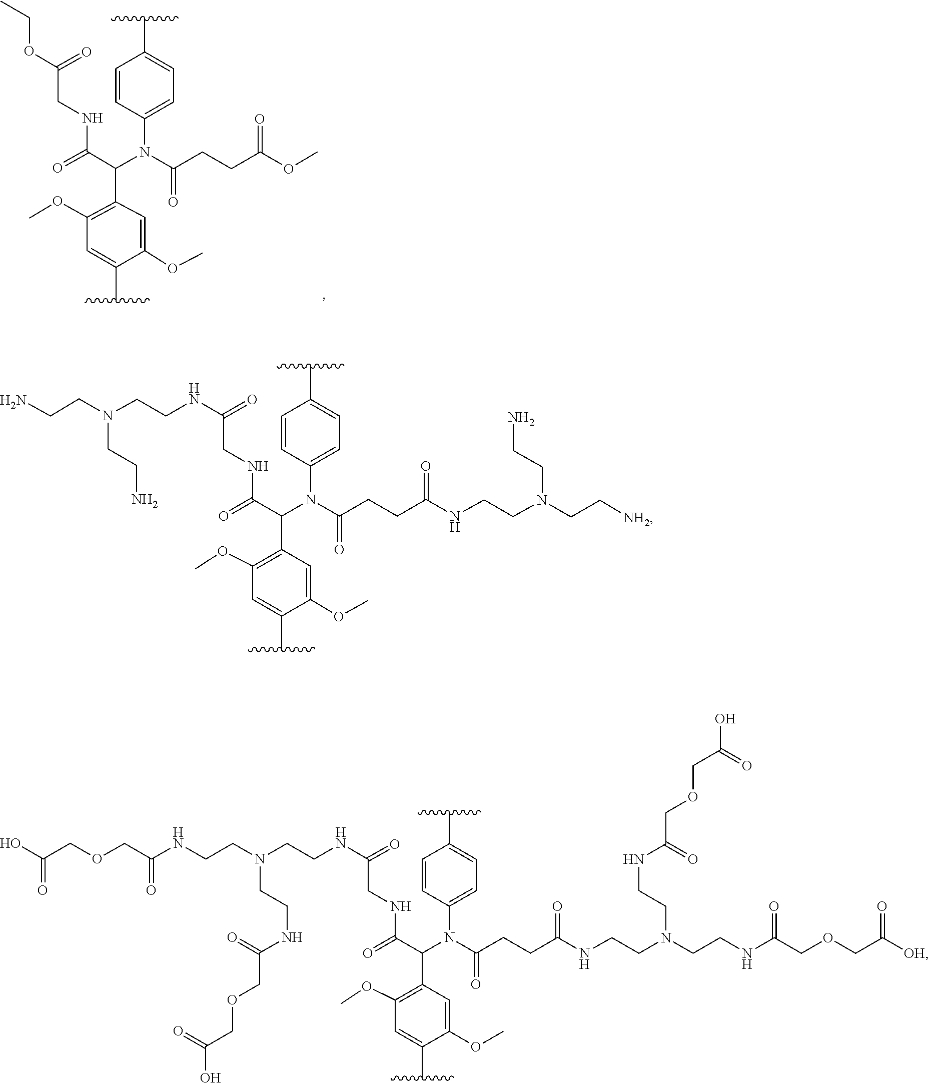

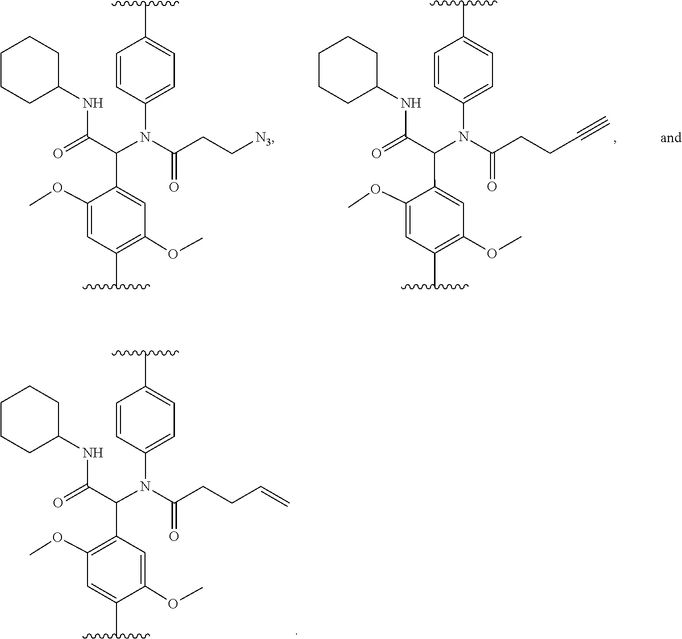

6. The covalent organic framework according to claim 1, wherein the repeating units of a moiety have a structure of Formula (Ia): 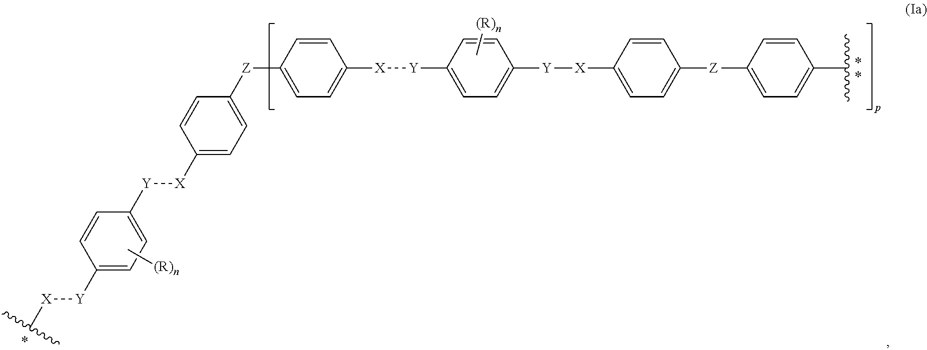
wherein 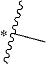
is the point of attachment of the moiety of Formula (Ia) to 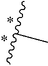
of another moiety of Formula (Ia);
Z is selected from the group consisting of C, CH, 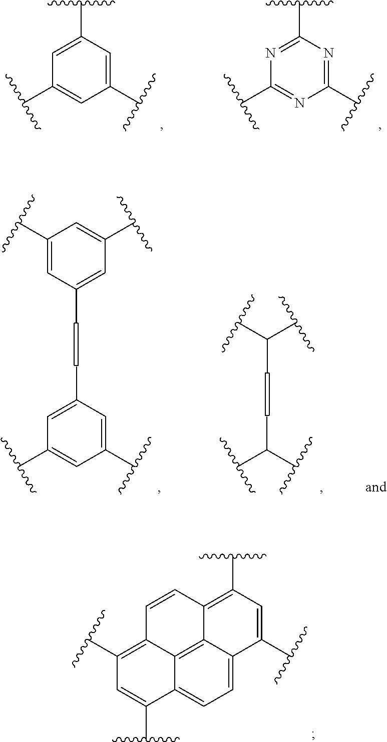
and
p is 2 or 3.

7. The covalent organic framework according to claim 1, wherein the repeating units of a moiety have a structure of Formula (Ib): 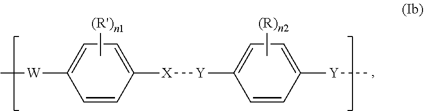
wherein
W is selected from the group consisting of X, 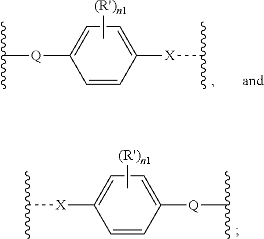
and
Q is optional, and if present, is —NHC(O)— or —C(O)NH—.

8. The covalent organic framework according to claim 1, wherein the covalent organic framework comprises a moiety of Formula (Ic):

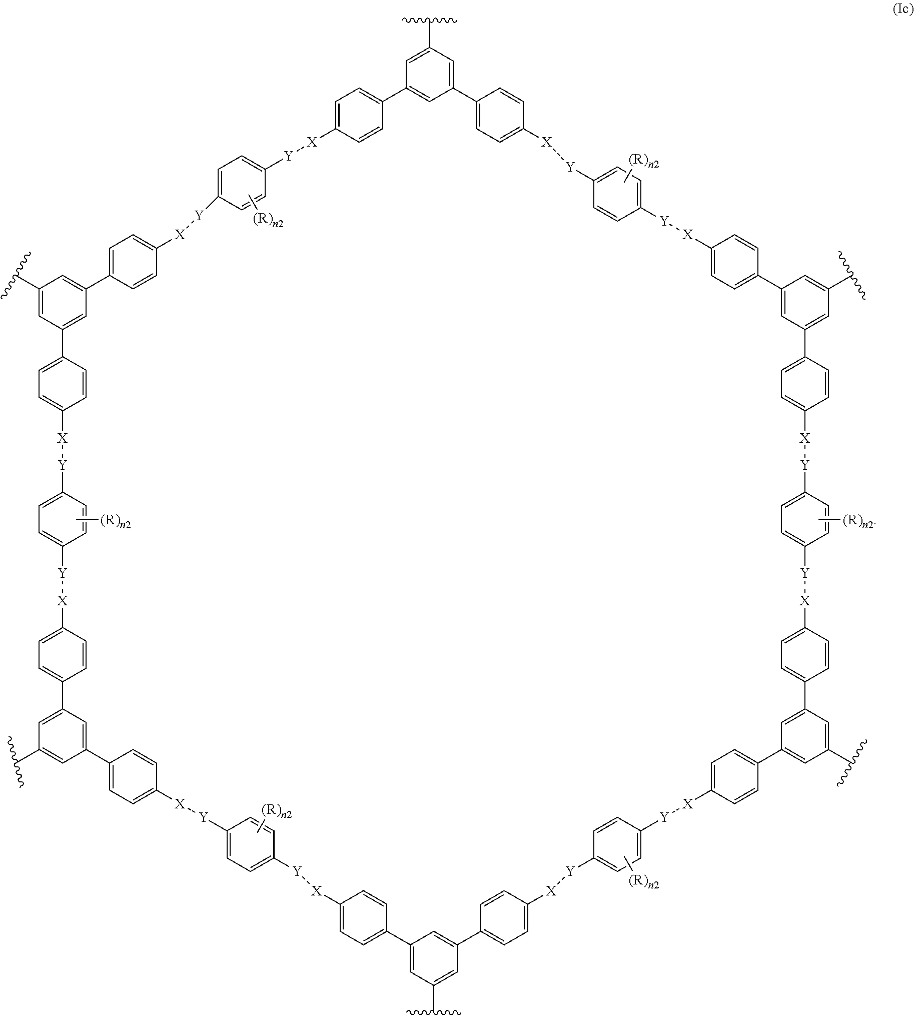

9. A method of separating rare-earth elements comprising:
providing a sample comprising a rare-earth element;
providing a covalent organic framework according to claim 1;
contacting the sample with the covalent organic framework under conditions effective to separate the rare-earth elements from the sample.

10. The method of claim 9, wherein the sample contains Nd(NO₃)₃.

11. The method of claim 9, wherein the sample contains two or more rare-earth elements.

12. The method of claim 9, wherein said contacting the sample with the covalent organic framework results in capture of the rare earth element inside the covalent organic framework.

13. The method of claim 12, wherein the sample contains two or more rare-earth elements.

14. The method of claim 13, wherein said contacting the sample with the covalent organic framework results in more effective capture of one rare-earth element over another rare-earth element.

15. The method of claim 14, wherein the sample contains Nd(NO₃)₃ and Dy(NO₃)₃.

16. The method of claim 15, wherein Dy ions are captured more effectively than Nd ions.

17. The method of claim 14, further comprising:
isolating the covalent organic framework containing the rare-earth elements from the sample.

18. The method of claim 17, wherein said isolating is carried out by filtration.

19.-20. (canceled)

21. A method of making a covalent organic framework comprising repeating units of a moiety comprising Formula (I): 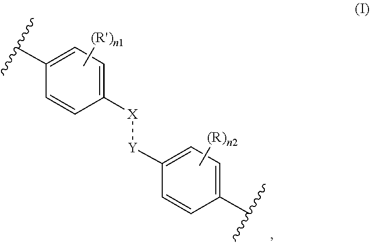
wherein
--- is a single or a double bond;
X is independently selected at each occurrence thereof from N or N(R³), wherein at least one X is N(R³);
Y is independently selected at each occurrence thereof from C(R¹)(R²) or C(R¹);
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R¹ is H;
R² is optional and, if present, is —C(O)NHR⁴;
R³ is —C(O)R⁵;
R⁴ is independently selected at each occurrence thereof from the group consisting of cycloalkyl, —(CH₂)ₘC(O)OC₁₋₆ alkyl, and —(CH₂)ₘC(O)NH(CH₂)ₖNH₂, wherein —(CH₂)ₘC(O)NH(CH₂)ₖNH₂ can be optionally substituted 1 to 3 times with R⁷;
R⁵ is selected from the group consisting of C₁₋₁₂ alkyl, C₂₋₁₂ alkenyl, or C₂₋₁₂ alkynyl, wherein C₁₋₁₂ alkyl can be optionally substituted 1 to 3 times with R⁶;
R⁶ is independently selected at each occurrence thereof from N₃, —C(O)OC₁₋₆ alkyl, —C(O)NH(CH₂)ₖNH₂, wherein —C(O)NH(CH₂)ₖNH₂ can be optionally substituted 1 to 3 times with R⁷;
R⁷ is —(CH₂)ₖ₁NH₂ optionally substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5, or 6; and
k₇ is 1, 2, 3, 4, 5, or 6;
said method comprises:
providing a covalent organic framework comprising repeating units of a moiety comprising Formula (II): 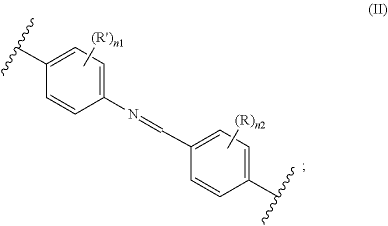
and
reacting the covalent organic framework comprising repeating units of a moiety comprising Formula (II) under conditions effective to produce the covalent organic framework.

22.-30. (canceled)

31. A method of making a modified covalent organic framework comprising repeating units of a moiety comprising Formula (I′a): 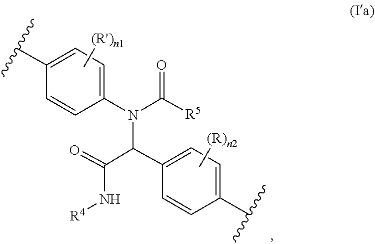
wherein
R is independently selected at each occurrence thereof from H or OC₁₋₆ alkyl;
R′ is independently selected at each occurrence thereof from H, OH, NO₂, heteroaryl, wherein heteroaryl can be optionally substituted 1 to 3 times with C₁₋₆ alkyl;
R⁴ is —(CH₂)ₘC(O)NH(CH₂)ₖNH₂ substituted 1 to 3 times with R⁷;
R⁵ is C₁₋₁₂ alkyl substituted with R⁶;
R⁶ is —C(O)NH(CH₂)ₖNH₂ substituted 1 to 3 times with R⁷;
R⁷ is —(CH₂)ₖ₁NH₂ substituted with —C(O)(CH₂)ₖ₂O(CH₂)ₖ₃C(O)OH, —C(O)(CH₂)ₖ₂N[(CH₂)ₖ₃C(O)OH](CH₂)ₖ₄N((CH₂)ₖ₅C(O)OH)₂, —C(O)(CH₂)ₖ₂N[(CH₂)ₖ₃C(O)OH](CH₂)ₖ₄N[(CH₂)ₖ₅C(O)OH](CH₂)ₖ₆N((CH₂)ₖ₇C(O)OH)₂, or —C(O)(CH₂)ₖ₂N((CH₂)ₖ₃P(O)(OH)₂)₂;
n₁ is 0, 1, 2, 3, or 4;
n₂ is 0, 1, 2, 3, or 4;
m is 1, 2, 3, 4, 5, or 6;
k is 1, 2, 3, 4, 5, or 6;
k₁ is 1, 2, 3, 4, 5, or 6;
k₂ is 1, 2, 3, 4, 5, or 6;
k₃ is 1, 2, 3, 4, 5, or 6;
k₄ is 1, 2, 3, 4, 5, or 6;
k₅ is 1, 2, 3, 4, 5, or 6;
k₆ is 1, 2, 3, 4, 5, or 6; and
k₇ is 1, 2, 3, 4, 5, or 6;
said method comprises:
providing a covalent organic framework comprising repeating units of a moiety comprising Formula (I′b): 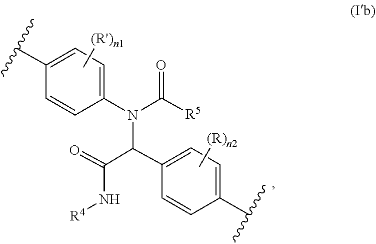
wherein
R⁴ is —(CH₂)ₘC(O)NH(CH₂)ₖNH₂ substituted 1 to 3 times with —(CH₂)ₖ₁NH₂;
R⁵ is C₁₋₁₂ alkyl substituted with R⁶; and
R⁶ is —C(O)NH(CH₂)ₖNH₂ substituted 1 to 3 times with —(CH₂)ₖ₁NH₂; and
reacting the covalent organic framework comprising repeating units of a moiety comprising Formula (I′b) under conditions effective to produce the modified covalent organic framework.

32.-40. (canceled)
